(19) United States
(12) Patent Application Publication
(10) Pub. No.: US 2014/0135384 A1
(43) Pub. Date: May 15, 2014

(54) ENDOGENOUS RETROVIRUS POLYPEPTIDES LINKED TO ONCOGENIC TRANSFORMATION

(71) Applicant: NOVARTIS VACCINES & DIAGNOSTICS, INC., Emeryville, CA (US)

(72) Inventors: Pablo GARCIA, Oakland, CA (US); Stephen F. HARDY, San Francisco, CA (US); Lewis T. WILLIAMS, Mill Valley, CA (US); Jaime ESCOBEDO, Alamo, CA (US)

(73) Assignee: NOVARTIS VACCINES & DIAGNOSTICS, INC., Emeryville, CA (US)

(21) Appl. No.: 14/156,167

(22) Filed: Jan. 15, 2014

Related U.S. Application Data
(62) Division of application No. 10/497,786, filed on Dec. 9, 2005.
Application No. 10/497,786 is a continuation-in-part of application No. 10/016,604, filed on Dec. 7, 2001, now Pat. No. 7,776,523.
(60) Provisional application No. 60/340,064, filed on Dec. 7, 2001.
(60) Provisional application No. 60/388,046, filed on Jun. 12, 2002.

(51) Int. Cl. C07K 14/005

(52) U.S. Cl. CPC C07K 14/005
USPC 514/44 R; 536/23.72

(57) ABSTRACT

HERV-K human endogenous retroviruses show up-regulated expression in tumors. In particular, splicing events in the env region generate a series of transcripts which utilize the +2 reading frame, relative to the env reading frame. The proteins show activity typical of transcriptional regulators, and they also have oncogenic potential. Two related proteins, PCAP2 and PCAP3, are strongly associated with breast cancer and prostate cancer, respectively. PCAP4 stimulates cell division. These proteins can be used in cancer diagnosis and therapy, and are also drug targets e.g. for adjuvant therapy. The identification of these splice products is remarkable because full sequence information has been available for HERV-K viruses since 1986.

### CROSS-REFERENCE TO RELATED APPLICATIONS

This Application is a Divisional of U.S. patent application Ser. No. 10/497,786, filed Jun. 7, 2004, which is a U.S. National Phase of International Patent Application No. PCT/US2002/039344, filed Dec. 9, 2002, which is a Continuation-in-Part of U.S. patent application Ser. No. 10/016,604, filed Dec. 7, 2001, now U.S. Pat. No. 7,776,523, issued Aug. 17, 2010, and claims the benefit of U.S. Provisional Patent Application No. 60/340,064, filed Dec. 7, 2001, and U.S. Provisional Patent Application No. 60/388,046, filed Jun. 12, 2002, all of which are incorporated herein by reference in their entirety.

### TECHNICAL FIELD

The present invention relates to the diagnosis of cancer e.g. prostate cancer. In particular, it relates to a subgroup of human endogenous retroviruses (HERVs) which show up-regulated expression in prostate tumors, and to the polypeptides encoded by spliced mRNAs expressed by these viruses.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 223002123310SeqList.txt, date recorded: Jan. 14, 2014, size: 215 KB).

### BACKGROUND ART

References 1 and 2 disclose that human endogenous retroviruses (HERVs) of the HML-2 subgroup of the HERV-K family show up-regulated expression in prostate tumors. The contents of references 1 and 2 are incorporated herein by reference.

It is an object of the invention to provide further materials that can be used in the prevention, treatment and diagnosis of cancer, e.g., prostate cancer. It is a further object to provide improvements in the prevention, treatment and diagnosis of cancer e.g. prostate cancer and breast cancer.

### DISCLOSURE OF THE INVENTION

HERVs have been known for many years, and genomic sequence for the HERV-K family has been known since 1986 {ref. 187}. The usual gag, prt, pol and env retroviral proteins have been identified for HERV-K, as has an analogue of IHV Rev or HTLV Rex, known as cORF or Rec {3}, but analogues of other regulatory proteins (e.g. HIV Tat or HTLV Tax proteins) have not been identified.

The Rev/Rex analog ‘cORF’ is encoded by an ORF which shares the same 5′ region and start codon as env, but in which a splicing event removes env-coding sequences and shifts to a reading frame +1 relative to that of env {4, 5}. Within the final exon in the env region of PCAV, therefore, reading frames 1 and 2 encode env and cORF, respectively, but no protein encoded by the third reading frame has previously been reported, and this +2 reading frame has no known function in HERV-K.

The inventors have now found a series of proteins generated by splicing in the env region of HERV-K genomes, including several which utilize the +2 reading frame. The proteins show activity typical of transcriptional regulators, and they also have oncogenic potential. These proteins can be used in cancer diagnosis and therapy, and are also drug targets e.g. for adjuvant therapy.

The identification of these new polypeptide products is remarkable because full sequence information has been available for HERV-K viruses for over 15 years.

The invention provides a method for diagnosing cancer, the method comprising the step of detecting the presence or absence in a patient sample of a HML-2 expression product produced by a splicing event in which the 5′ region and start codon of the env coding region are joined to a downstream coding region in the reading frame +2 relative to that of env in the genome. Higher levels of expression product relative to normal tissue indicate that the patient from whom the sample was taken has cancer (e.g. prostate cancer). The expression product may or may not be functional in a viral life cycle.

The expression product which is detected is either a mRNA transcript or a polypeptide translated from such a transcript. These expression products may be detected directly or indirectly. A direct test uses an assay which detects HML-2 RNA or polypeptide in a patient sample. An indirect test uses an assay which detects biomolecules which are not directly expressed in vivo from HML-2 e.g. an assay to detect cDNA which has been reverse-transcribed from a HML-2 mRNA, or an assay to detect an antibody which has been raised in response to a HML-2 polypeptide.

### A—the Patient Sample

Where the diagnostic method of the invention is based on mRNA for diagnosis of cancer, the patient sample will generally comprise cells from the tissue of interest e.g. prostate cells for prostate cancer, breast cells for breast cancer, etc. These cells may be present in a sample of tissue taken from the relevant organ, or may be cells which have escaped into circulation (e.g. during metastasis). Instead of or as well as comprising cells, the sample may comprise virions which contain mRNA from HML-2, or bodily fluids.

Where the diagnostic method of the invention is based on polypeptide, the patient sample may comprise cells and/or virions (as described above for mRNA), or may comprise antibodies which recognize the polypeptide. Such antibodies will typically be present in circulation.

In general, therefore, the patient sample for males is a prostate sample (e.g. a biopsy) or a blood sample, and for females it is a breast sample (e.g. a biopsy) or a blood sample.

The patient is generally a human, and preferably an adult human.

Expression products may be detected in the patient sample itself, or may be detected in material derived from the sample (e.g. the supernatant of a cell lysate, or a RNA extract, or cDNA generated from a RNA extract, or polypeptides translated from a RNA extract, or cells derived from culture of cells extracted from a patient, etc.). These are still considered to be “patient samples” within the meaning of the invention.

Methods of the invention can be conducted in vitro or in vivo.

Other possible sources of patient samples include isolated cells, whole tissues, or bodily fluids (e.g. blood, plasma, serum, urine, pleural effusions, cerebro-spinal fluid, breast milk, colostrum, other fluids secreted by the breast, semen, seminal fluid, etc.)

### B—the mRNA Expression Product

Where the diagnostic method of the invention is based on mRNA detection, it typically involves detecting a RNA which encodes a polypeptide of the invention. The RNA will comprise the ATG codon of the Env ORF which, through splicing as shown in FIG. 17, is in the same reading frame as sequences from the 3′ end of the Env ORF, but which are (relative to the ATG in the genomic DNA copy of HML-2) in the +2 reading frame (i.e. the third reading frame). The invention may thus involve a step of detecting a RNA produced by a splicing event in which the 5′ region and start codon of the env coding region are joined to a downstream coding region in the reading frame +2 relative to that of env.

Preferred RNAs comprise a sequence which has at least s % sequence identity to SEQ ID 52. SEQ ID 52 is the 50 nucleotides of the HERV-K(C7) virus {ref. 6} immediately downstream of ‘Potential splice site B’ in FIG. 22.

Other preferred RNAs comprise a sequence which has at least s % sequence identity to one or more of SEQ IDs 19, 20, 21, 24, 25, 26, 38, 40 and/or 42. Particularly preferred RNAs comprise a sequence which has at least s % sequence identity to one or more of SEQ IDs 38, 40 and/or 42.

Preferred RNAs comprise a sequence which encodes a polypeptide having at least s % sequence identity to one or more of SEQ IDs 7, 8, 9, 10, 11, 21, 28, 29, 30, 31, 34, 35, 36, 39, 41, 43, 67, 68 and 69. Particularly preferred RNAs comprise a sequence which encodes a polypeptide having at least s % sequence identity to one or more of SEQ IDs 7, 8 and/or 9.

The value of s is preferably at least 50 (e.g. at least 55, 60, 65, 70, 75, 80, 85, 90, 91, 92, 93, 94, 95, 96, 97, 98, 99, 99.5, 99.9, etc.).

Preferred RNAs encode a polypeptide which may bind to RNA comprising SEQ ID 49.

The RNA will usually also comprise one, two, three, four or five of the following:
1. An upstream sequence which has at least 75% identity to SEQ ID 49 (e.g. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity); or a sequence which has at least 50% identity to SEQ ID 49 (e.g. 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) and is expressed at least 1.5 fold (e.g. 2, 2.5, 5, 10, 20, 50, etc., fold) higher level relative to expression in a normal (i.e., non cancerous) cell with at least a 95% confidence level; or a sequence which has at least 80% identity (e.g. 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) to at least a 20 contiguous nucleotide fragment (e.g. 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, etc., contiguous nucleotides) of SEQ ID 49; or a sequence which has at least 80% identity (e.g. 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) to at least a 20 contiguous nucleotide fragment (e.g. 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, etc., contiguous nucleotides) of SEQ ID 49 and is expressed at least 1.5 fold (e.g. 2, 2.5, 5, 10, 20, 50, etc., fold) higher level relative to expression in a normal (i.e., non cancerous) cell with at least a 95% confidence level. This sequence will typically be at the 5′ end of the RNA. SEQ ID 49 is the nucleotide sequence of the start of R region in the LTR of the ‘ERVK6’ HML-2 virus {ref. 7}. This portion of the R region may be found in HML-2 transcripts, and transcription of mRNA molecules including this portion of the R region is up-regulated in prostate cancer.
2. An upstream region comprising a sequence which has at least 75% sequence identity to SEQ ID 50 (e.g. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity); or a sequence which has at least 50% identity to SEQ ID 50 (e.g. 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) and is expressed at least 1.5 fold (e.g. 2, 2.5, 5, 10, 20, 50, etc., fold) higher level relative to expression in a normal (i.e., non cancerous) cell with at least a 95% confidence level; or a sequence which has at least 80% identity (e.g. 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) to at least a 20 contiguous nucleotide fragment (e.g. 25, 30, 35, 40, 45, 50, 55, 60, 65, 70, 75, 80, 85, 90, 95, 100, 110, 115, 120, 125, 130, 135, 140, 145, etc., contiguous nucleotides) of SEQ ID 50; or a sequence which has at least 80% identity (e.g. 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) to at least a 20 contiguous nucleotide fragment (e.g. 25, 30, 35, 40, 45, 50, 55, 60, 65, 70, 75, 80, 85, 90, 95, 100, 110, 115, 120, 125, 130, 135, 140, 145, etc., contiguous nucleotides) of SEQ ID 50 and is expressed at least 1.5 fold (e.g. 2, 2.5, 5, 10, 20, 50, etc., fold) higher level relative to expression in a normal (i.e., non cancerous) cell with at least a 95% confidence level. SEQ ID 50 is the nucleotide sequence of the RU₅ region downstream of SEQ ID 49 in the ERVK6 LTR. This region is found in full-length HML-2 transcripts, but may not be present in all mRNAs transcribed from a HML-2 LTR promoter (e.g. if transcription is attenuated).
3. An upstream region comprising a sequence which has at least 75% sequence identity to SEQ ID 6 (e.g. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity); or a sequence which has at least 50% identity to SEQ ID 6 (e.g. 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) and is expressed at least 1.5 fold (e.g. 2, 2.5, 5, 10, 20, 50, etc., fold) higher level relative to expression in a normal (i.e., non cancerous) cell with at least a 95% confidence level; or a sequence which has at least 80% identity (e.g. 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) to at least a 20 contiguous nucleotide fragment (e.g. 25, 30, 35, 40, 45, 50, 55, 60, 65, 70, 75, 80, 85, 90, 95, 100, etc., contiguous nucleotides) of SEQ ID 6; or a sequence which has at least 80% identity (e.g. 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) to at least a 20 contiguous nucleotide fragment (e.g. 25, 30, 35, 40, 45, 50, 55, 60, 65, 70, 75, 80, 85, 90, 95, 100, etc., contiguous nucleotides) of SEQ ID 6 and is expressed at least 1.5 fold (e.g. 2, 2.5, 5, 10, 20, 50, etc., fold) higher level relative to expression in a normal (i.e., non cancerous) cell with at least a 95% confidence level. SEQ ID 6 is the nucleotide sequence of the region of the ERVK6 virus between the U₅ region and the first 5′ splice site. This region is found in full-length HML-2 transcripts, but has been lost by some variants and, like region 2 above, may not be present in all mRNAs transcribed from a HML-2 LTR promoter.
4. A downstream region comprising a sequence which has at least 75% sequence identity to SEQ ID 5 (e.g. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity); or a sequence which has at least 50% identity to SEQ ID 5 (e.g. 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) and is expressed at least 1.5 fold (e.g. 2, 2.5, 5, 10, 20, 50, etc., fold) higher level relative to expression in a normal (i.e., non cancerous) cell with at least a 95% confidence level; or a sequence which has at least 80% identity (e.g. 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) to at least a 20 contiguous nucleotide fragment (e.g. 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, etc., contiguous nucleotides) of SEQ ID 5; or a sequence which has at least 80% identity (e.g. 81%, 82%, 83%, 84%, 85%, 86%, 87%, 88%, 89%, 90%, 91%, 92%, 93%, 94%, 95%, 96%, 97%, 98%, 99%, 99.5%, 99.9%, 100% identity) to at least a 20 contiguous nucleotide fragment (e.g. 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, etc., contiguous nucleotides) of SEQ ID 5 and is expressed at least 1.5 fold (e.g. 2, 2.5, 5, 10, 20, 50, etc., fold) higher level relative to expression in a normal (i.e., non cancerous) cell with at least a 95% confidence level. SEQ ID 5 is the nucleotide sequence of the U₃R region in the 3′ end of ERVK6. This sequence will typically be between the stop codon of the Tat-coding region and immediately precedes any polyA tail.
5. A downstream 3′ polyA tail.

The percent identity of the sequences described above are determined by the Smith-Waterman algorithm using the default parameters: open gap penalty=−20 and extension penalty=−5.

These mRNA molecules are referred to below as “PCA-mRNA” molecules (“prostate cancer associated mRNA”), and endogenous viruses which express these PCA-mRNAs are referred to as PCAVs (“prostate cancer associated viruses”). Nevertheless, said PCAVs may also be associated with other types of cancer and, in particular, breast cancer.

In general, therefore, the mRNA to be detected has formula N₁-N₂-N₃-N₄-N₅-polyA, wherein:
N₁ has at least 75% sequence identity to SEQ ID 49; or has at least 50% identity to SEQ ID 49 and is expressed at least 1.5 fold higher relative to expression in a normal (i.e., non cancerous) cell with at least a 95% confidence level; or has at least 80% identity to at least a 20 contiguous nucleotide fragment of SEQ ID 49; or has at least 80% identity to at least a 20 contiguous nucleotide fragment of SEQ ID 49 and is expressed at least 1.5 fold higher relative to expression in a normal (i.e., non cancerous) cell with at least a 95% confidence level;
N₂ has at least 75% sequence identity to SEQ ID 50; or has at least 50% identity to SEQ ID 50 and is expressed at least 1.5 fold higher relative to expression in a normal (i.e., non cancerous) cell with at least a 95% confidence level; or has at least 80% identity to at least a 20 contiguous nucleotide fragment of SEQ ID 50; or has at least 80% identity to at least a 20 contiguous nucleotide fragment of SEQ ID 50 and is expressed at least 1.5 fold higher relative to expression in a normal (i.e., non cancerous) cell with at least a 95% confidence level;
N₃ has at least 75% sequence identity to SEQ ID 6; or has at least 50% identity to SEQ ID 6 and is expressed at least 1.5 fold higher relative to expression in a normal (i.e., non cancerous) cell with at least a 95% confidence level; or has at least 80% identity to at least a 20 contiguous nucleotide fragment of SEQ ID 6; or has at least 80% identity to at least a 20 contiguous nucleotide fragment of SEQ ID 6 and is expressed at least 1.5 fold higher relative to expression in a normal (i.e., non cancerous) cell with at least a 95% confidence level
N₄ comprises a RNA sequence which includes the start codon of the env coding region spliced to a downstream coding region in the reading frame +2 relative to that of env;
N₅ comprises a sequence which has at least 75% sequence identity to SEQ ID 5; or has at least 50% identity to SEQ ID 5 and is expressed at least 1.5 fold higher relative to expression in a normal (i.e., non cancerous) cell with at least a 95% confidence level; or has at least 80% identity to at least a 20 contiguous nucleotide fragment of SEQ ID 5; or has at least 80% identity to at least a 20 contiguous nucleotide fragment of SEQ ID 5 and is expressed at least 1.5 fold higher relative to expression in a normal (i.e., non cancerous) cell with at least a 95% confidence level; and
N₁ and N₄ are present, but N₂, N₃, N₅ and polyA are optional. N₁ is present in the mRNA to be detected and, more preferably, N₁-N₂ is present.

N₁ is preferably at the 5′ end of the mRNA (i.e. 5′-N₁- . . . ). Although N₁ is defined above by reference to SEQ ID 49, up to 100 nucleotides (e.g. 10, 20, 30, 40, 50, 60, 70, 71, 72, 73, 74, 75, 76, 77, 78, 79, 80, 81, 82, 83, 84, 85, 86, 87, 88, 89, 90 or 100) from the 5′ end of SEQ ID 49 may be omitted, depending on the start site of transcription e.g. N₁ may at least 75% sequence identity to SEQ ID 478.

Where N₅ is present, it is preferably immediately before a 3′ polyA tail (i.e. . . . N₅-polyA-3′).

The RNA will generally have a 5′ cap.

### B.1—Enriching RNA in a Sample

Where diagnosis is based on mRNA detection, the method of the invention preferably comprises an initial step of: (a) extracting RNA (e.g. mRNA) from a patient sample; (b) removing DNA from a patient sample without removing mRNA; and/or (c) removing or disrupting DNA comprising SEQ ID 4, but not RNA comprising SEQ ID 4, from a patient sample. This is necessary because the genomes of both normal and cancerous cells contain multiple PCAV DNA templates, whereas increased PCA-mRNA levels are only found in cancerous cells. As an alternative, a RNA-specific assay can be used which is not affected by the presence of homologous DNA.

Methods for extracting RNA from biological samples are well known {e.g. refs. 8 & 17} and include methods based on guanidinium buffers, lithium chloride, SDS/potassium acetate etc. After total cellular RNA has been extracted, mRNA may be enriched e.g. using oligo-dT techniques.

Methods for removing DNA from biological samples without removing mRNA are well known {e.g. appendix C of ref. 8} and include DNase digestion.

Methods for removing DNA, but not RNA, comprising PCA-mRNA sequences will use a reagent which is specific to a sequence within a PCA-mRNA e.g. a restriction enzyme which recognizes a DNA sequence within SEQ 11) 4, but which does not cleave the corresponding RNA sequence.

Methods for specifically purifying PCA-mRNAs from a sample may also be used. One such method uses an affinity support which binds to PCA-mRNAs. The affinity support may include a polypeptide sequence which binds to the LTR of PCAV e.g. the tat polypeptide described below.

### B.2—Direct Detection of RNA

Various techniques are available for detecting the presence or absence of a particular RNA sequence in a sample {e.g. refs. 8 & 17}. If a sample contains genomic PCAV DNA, the detection technique will generally be RNA-specific; if the sample contains no PCAV DNA, the detection technique may or may not be RNA-specific.

Hybridization-based detection techniques may be used, in which a polynucleotide probe complementary to a region of PCA-mRNA is contacted with a RNA-containing sample under hybridizing conditions. Detection of hybridization indicates that nucleic acid complementary to the probe is present. Hybridization techniques for use with RNA include Northern blots, in situ hybridization and arrays.

Sequencing may also be used, in which the sequence(s) of RNA molecules in a sample are obtained. These techniques reveal directly whether a sequence of interest is present in a sample. Sequence determination of the 5′ end of a RNA corresponding to N₁ will generally be adequate.

Amplification-based techniques may also be used. These include PCR, SDA, SSSR, LCR, TMA, NASBA, T7 amplification etc. The technique preferably gives exponential amplification. A preferred technique for use with RNA is RT-PCR {e.g. see chapter 15 of ref. 8}. RT-PCR of mRNA from prostate cells is reported in references 9, 10, 11, 12, etc., and RT-PCT of mRNA from breast cells is reported in references 13, 14, 15, 16, etc.

### B.3—Indirect Detection of RNA

Rather than detect RNA directly, it may be preferred to detect molecules which are derived from RNA (i.e. indirect detection of RNA). A typical indirect method of detecting mRNA is to prepare cDNA by reverse transcription and then to directly detect the cDNA. Direct detection of cDNA will generally use the same techniques as described above for direct detection of RNA (but it will be appreciated that methods such as RT-PCR are not suitable for DNA detection and that cDNA is double-stranded, so detection techniques can be based on a sequence, on its complement, or on the double-stranded molecule).

### B.4—Polynucleotide Materials

The invention provides polynucleotide materials e.g. for use in the detection of PCAV nucleic acids.

The invention provides an isolated polynucleotide comprising: (a) the nucleotide sequence N₁-N₂-N₃-N₄-N₅-polyA as defined above; (b) a fragment of at least x nucleotides of nucleotide sequence N₁-N₂-N₃-N₄-N₅ as defined above; (c) a nucleotide sequence having at least s % identity to nucleotide sequence N₁-N₂-N₃-N₄-N₅ as defined above; or (d) the complement of (a), (b) or (c). These polynucleotides include variants of nucleotide sequence N₁-N₂-N₃-N₄-N₅-polyA (e.g. degenerate variants, allelic variants, homologs, orthologs, mutants, etc.).

Fragment (b) preferably comprises a fragment of N₄.

The value of x is at least 7 (e.g. at least 8, 9, 10, 11, 12, 13, 14, 15, 16, 17, 18, 19, 20, 21, 22, 23, 24, 25, 30, 35, 40, 45, 50, 60, 70, 75, 80, 90, 100 etc.). The value of x may be less than 2000 (e.g. less than 1000, 500, 100, or 50).

The value of s is preferably at least 50 (e.g. at least 55, 60, 65, 70, 75, 80, 85, 90, 91, 92, 93, 94, 95, 96, 97, 98, 99, 99.5, 99.9 etc.).

The invention also provides an isolated polynucleotide having formula 5′-A-B-C-3′, wherein: -A- is a nucleotide sequence consisting of a nucleotides; -C- is a nucleotide sequence consisting of c nucleotides; -B- is a nucleotide sequence consisting of either (a) a fragment of b nucleotides of nucleotide sequence N₁-N₂-N₃-N₄-N₅ as defined above or (b) the complement of a fragment of b nucleotides of nucleotide sequence N₁-N₂-N₃-N₄-N₅ as defined above; and said polynucleotide is neither (a) a fragment of nucleotide sequence N₁-N₂-N₃-N₄-N₅ or (b) the complement of a fragment of nucleotide sequence N₁-N₂-N₃-N₄-N₅.

The -B- region is preferably a fragment of N₄. The -A- and/or -C- portions may comprise a promoter sequence (or its complement) e.g. for use in TMA.

The value of a+c is at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100 etc.). The value of b is at least 7 (e.g. at least 8, 9, 10, 11, 12, 13, 14, 15, 16, 17, 18, 19, 20, 21, 22, 23, 24, 25, 30, 35, 40, 45, 50, 60, 70, 80, 90, 100 etc.). It is preferred that the value of a+b+c is at least 9 (e.g. at least 10, 11, 12, 13, 14, 15, 16, 17, 18, 19, 20, 21, 22, 23, 24, 25, 30, 35, 40, 45, 50, 60, 70, 80, 90, 100 etc.). It is preferred that the value of a+b+c is at most 500 (e.g. at most 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9).

Where -B- is a fragment of N₁-N₂-N₃-N₄-N₅, the nucleotide sequence of -A- typically shares less than n % sequence identity to the a nucleotides which are 5′ of sequence -B- in N₁-N₂-N₃-N₄-N₅ and/or the nucleotide sequence of -C- typically shares less than n % sequence identity to the c nucleotides which are 3′ of sequence -C- in N₁-N₂-N₃-N₄-N₅. Similarly, where -B- is the complement of a fragment of N₁-N₂-N₃-N₄-N₅, the nucleotide sequence of -A- typically shares less than n % sequence identity to the complement of the a nucleotides which are 5′ of the complement of sequence -B- in N₁-N₂-N₃-N₄-N₅ and/or the nucleotide sequence of -C- typically shares less than n % sequence identity to the complement of the c nucleotides which are 3′ of the complement of sequence -C- in N₁-N₂-N₃-N₄-N₅. The value of n is generally 60 or less (e.g. 50, 40, 30, 20, 10 or less).

The invention also provides an isolated polynucleotide which selectively hybridizes to a nucleic acid having nucleotide sequence N₁-N₂-N₃-N₄-N₅ as defined above or to a nucleic acid having the complement of nucleotide sequence N₁-N₂-N₃-N₄-N₅ as defined above. The polynucleotide preferably hybridizes to at least to N₄.

Hybridization reactions can be performed under conditions of different “stringency”. Conditions that increase stringency of a hybridization reaction of widely known and published in the art {e.g. page 7.52 of reference 17}. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques are well known in the art {e.g. see references 8, 17, 18, 19, 20 etc.}. Depending upon the particular polynucleotide sequence and the particular domain encoded by that polynucleotide sequence, hybridization conditions upon which to compare a polynucleotide of the invention to a known polynucleotide may differ, as will be understood by the skilled artisan.

In some embodiments, the isolated polynucleotide of the invention selectively hybridizes under low stringency conditions; in other embodiments it selectively hybridizes under intermediate stringency conditions; in other embodiments, it selectively hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringent hybridization conditions is 68° C. and 0.1×SSC.

Particularly preferred polynucleotides of the invention encode a polypeptide as defined below. By “encode”, it is not necessarily implied that the polynucleotide (e.g. RNA) is translated, but it will include a series of codons which encode the amino acids of the polypeptides defined below.

The invention also provides a polynucleotide comprising: (a) a nucleotide sequence selected from the group consisting of SEQ IDs 278 to 477; (b) a fragment of at least x nucleotides of (a); (c) a nucleotide sequence having at least s % identity to (a); or (d) the complement of (a), (b) or (c).

The invention also provides a polynucleotide comprising: (a) a nucleotide sequence selected from the group consisting of SEQ IDs 18, 19, 20, 21, 22, 23, 24, 25, 26, 27, 38, 40, 42, 51 and 52; (b) a fragment of at least x nucleotides of (a); (c) a nucleotide sequence having at least s % identity to (a); or (d) the complement of (a), (b) or (c).

The polynucleotides of the invention are particularly useful as probes and/or as primers for use in hybridization and/or amplification reactions.

More than one polynucleotide of the invention can hybridize to the same nucleic acid target (e.g. more than one can hybridize to a single RNA).

References to a percentage sequence identity between two nucleic acid sequences mean that, when aligned, that percentage of bases are the same in comparing the two sequences. This alignment and the percent homology or sequence identity can be determined using software programs known in the art, for example those described in section 7.7.18 of reference 20. A preferred alignment program is GCG Gap (Genetics Computer Group, Wisconsin, Suite Version 10.1), preferably using default parameters, which are as follows: open gap=3; extend gap=1.

Polynucleotides of the invention may take various forms e.g. single-stranded, double-stranded, linear, circular, vectors, primers, probes etc.

Polynucleotides of the invention can be prepared in many ways e.g. by chemical synthesis (at least in part), by digesting longer polynucleotides using restriction enzymes, from genomic or cDNA libraries, from the organism itself etc.

Polynucleotides of the invention may be attached to a solid support (e.g. a bead, plate, filter, film, slide, resin, etc.)

Polynucleotides of the invention may include a detectable label (e.g. a radioactive or fluorescent label, or a biotin label). This is particularly useful where the polynucleotide is to be used in nucleic acid detection techniques e.g. where the nucleic acid is a primer or as a probe for use in techniques such as PCR, LCR, TMA, NASBA, bDNA, etc.

The term “polynucleotide” in general means a polymeric form of nucleotides of any length, which contain deoxyribonucleotides, ribonucleotides, and/or their analogs. It includes DNA, RNA, DNA/RNA hybrids, and DNA or RNA analogs, such as those containing modified backbones or bases, and also peptide nucleic acids (PNA) etc. The term “polynucleotide” is not intended to be limiting as to the length or structure of a nucleic acid unless specifically indicated, and the following are non-limiting examples of polynucleotides: a gene or gene fragment, exons, introns, mRNA, tRNA, rRNA, ribozymes, cDNA, recombinant polynucleotides, branched polynucleotides, plasmids, vectors, any isolated DNA from any source, any isolated RNA from any sequence, nucleic acid probes, and primers. Polynucleotides may have any three-dimensional structure, and may perform any function, known or unknown. Unless otherwise specified or required, any embodiment of the invention that includes a polynucleotide encompasses both the double-stranded form and each of two complementary single-stranded forms known or predicted to make up the double stranded form.

Polynucleotides of the invention may be isolated and obtained in substantial purity, generally as other than an intact chromosome. Usually, the polynucleotides will be obtained substantially free of other naturally-occurring nucleic acid sequences, generally being at least about 50% (by weight) pure, usually at least about 90% pure.

Polynucleotides of the invention (particularly DNA) are typically “recombinant” e.g. flanked by one or more nucleotides with which it is not normally associated on a naturally-occurring chromosome.

The polynucleotides can be used, for example: to produce polypeptides; as probes for the detection of nucleic acid in biological samples; to generate additional copies of the polynucleotides; to generate ribozymes or antisense oligonucleotides; and as single-stranded DNA probes or as triple-strand forming oligonucleotides. The polynucleotides are preferably uses to detect PCA-mRNAs.

A “vector” is a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector.

A “host cell” includes an individual cell or cell culture which can be or has been a recipient of exogenous polynucleotides. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a polynucleotide of this invention.

### B.5—Nucleic Acid Detection Kits

The invention provides a kit comprising primers (e.g. PCR primers) for amplifying a template sequence contained within a PCAV nucleic acid, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label.

The invention also provides a kit comprising first and second single-stranded oligonucleotides which allow amplification of a PCAV template nucleic acid sequence contained in a single- or double-stranded nucleic acid (or mixture thereof), wherein: (a) the first oligonucleotide comprises a primer sequence which is substantially complementary to said template nucleic acid sequence; (b) the second oligonucleotide comprises a primer sequence which is substantially complementary to the complement of said template nucleic acid sequence; (c) the first oligonucleotide and/or the second oligonucleotide comprise(s) sequence which is not complementary to said template nucleic acid; and (d) said primer sequences define the termini of the template sequence to be amplified. The non-complementary sequence(s) of feature (c) are preferably upstream of (i.e. 5′ to) the primer sequences. One or both of the (c) sequences may comprise a restriction site {21} or promoter sequence {22}. The first and/or the second oligonucleotide may include a detectable label.

The kit of the invention may also comprise a labeled polynucleotide which comprises a fragment of the template sequence (or its complement). This can be used in a hybridization technique to detect amplified template.

The primers and probes used in these kits are preferably polynucleotides as described in section B.4.

The target is preferable a polynucleotide sequence as defined in section B.1.

### C—Polypeptide Expression Products

Where the method is based on polypeptide detection, it will involve detecting expression of a polypeptide which is encoded by a transcript produced by a splicing event in which the 5′ region and start codon of the env coding region are joined to a downstream coding region in the reading frame +2 relative to that of env in the genome. The polypeptide may or may not be functional in a viral life cycle.

Transcripts which encode HML-2 polypeptides are generated by alternative splicing of the full-length mRNA copy of the endogenous genome {e.g. FIG. 4 of ref. 195; FIG. 17 herein}.

The polypeptides of the invention are encoded by ORFs which share the same 5′ region (and start codon) as env. A splicing event removes env-coding sequences, but the coding sequence continues in the reading frame +2 relative to that of env. Examples of spliced nucleotide sequences are: SEQ IDs 18-27, 38, 40 & 42. Examples of encoded polypeptide sequences are: SEQ IDs 7-12 and SEQ IDs 28, 29, 30, 31, 34, 35, 36, 39, 41, 43, 67, 68 and 69. Some of these (e.g. SEQ IDs 10-12) inhibit the function of PCAP4 in a transdominant fashion.

### C.1—Direct Detection of HML-2 Polypeptides

Various techniques are available for detecting the presence or absence of a particular polypeptides in a sample. These are generally immunoassay techniques which are based on the specific interaction between an antibody and an antigenic amino acid sequence in the polypeptide. Suitable techniques include standard immunohistological methods, immunoprecipitation, ELISA, RIA, FIA, immunofluorescence etc.

In general, therefore, the invention provides a method for detecting the presence of and/or measuring a level of Tat polypeptide of the invention in a biological sample, wherein the method uses an antibody specific for the polypeptide. The method generally comprises the steps of: a) contacting the sample with an antibody specific for the polypeptide; and b) detecting binding between the antibody and polypeptides in the sample.

Polypeptides of the invention can also be detected by functional assays e.g. assays to detect binding activity or enzymatic activity. For instance, transcriptionally-active polypeptides of the invention can be assayed by detecting expression of a reporter gene driven by the PCAV LTR, as described in the examples herein.

Another way for detecting polypeptides of the invention is to use standard proteomics techniques e.g. purify or separate polypeptides and then use peptide sequencing. For example, polypeptides can be separated using 2D-PAGE and polypeptide spots can be sequenced (e.g. by mass spectroscopy) in order to identify if a sequence is present in a target polypeptide.

Detection methods may be adapted for use in vivo (e.g. to locate or identify sites where cancer cells are present). In these embodiments, an antibody specific for a target polypeptide is administered to an individual (e.g. by injection) and the antibody is located using standard imaging techniques (e.g. magnetic resonance imaging, computed tomography scanning, etc.). Appropriate labels (e.g. spin labels etc.) will be used. Using these techniques, cancer cells are differentially labeled.

An immunofluorescence assay can be easily performed on cells without the need for purification of the target polypeptide. The cells are first fixed onto a solid support, such as a microscope slide or microliter well. The membranes of the cells are then permeablized in order to permit entry of polypeptide-specific antibody (NB: fixing and permeabilization can be achieved together). Next, the fixed cells are exposed to an antibody which is specific for the encoded polypeptide and which is fluorescently labeled. The presence of this label (e.g. visualized under a microscope) identifies cells which express the target PCAV polypeptide. To increase the sensitivity of the assay, it is possible to use a second antibody to bind to the anti-PCAV antibody, with the label being carried by the second antibody. {23}

### C.2—Indirect Detection of HML-2 Polypeptides

Rather than detect polypeptides directly, it may be preferred to detect molecules which are produced by the body in response to them (i.e. indirect detection of a polypeptide). This will typically involve the detection of antibodies, so the patient sample will generally be a blood sample. Antibodies can be detected by conventional immunoassay techniques e.g. using PCAV polypeptides of the invention, which will typically be immobilized.

Antibodies against HERV-K polypeptides have been detected in humans {195}.

### C.3—Polypeptide Materials

The invention provides an isolated polypeptide comprising: (a) an amino acid sequence selected from the group consisting of SEQ IDs 7, 8, 9, 10, 11, 12, 28, 29, 30, 31, 34, 35, 36, 39, 41, 43, 67, 68 and 69; (b) a fragment of at least x amino acids of (a); or (c) a polypeptide sequence having at least s % identity to (a). These polypeptides include variants (e.g. allelic variants, homologs, orthologs, functional and non-functional mutants etc.).

The value of x is at least 5 (e.g. at least 6, 7, 8, 9, 10, 11, 12, 13, 14, 15, 16, 17, 18, 19, 20, 21, 22, 23, 24, 25, 30, 35, 40, 45, 50, 60, 70, 75, 80, 90, 100 etc.). The value of x may be less than 2000 (e.g. less than 1000, 500, 100, or 50).

The value of s is preferably at least 50 (e.g. at least 55, 60, 65, 70, 75, 80, 85, 90, 91, 92, 93, 94, 95, 96, 97, 98, 99, 99.5, 99.9 etc.).

The invention also provides an isolated polypeptide having formula NH₂-A-B-C-COOH, wherein: A is a polypeptide sequence consisting of a amino acids; C is a polypeptide sequence consisting of c amino acids; B is a polypeptide sequence consisting of a fragment of b amino acids of an amino acid sequence selected from the group consisting of SEQ IDs 7, 8, 9, 10, 11, 12, 28, 29, 30, 31, 34, 35, 36, 39, 41, 43, 67, 68 and 69; and said polypeptide is not a fragment of polypeptide sequence SEQ ID 7, 8, 9, 10, 11, 12, 28, 29, 30, 31, 34, 35, 36, 39, 41, 43, 67, 68 or 69.

The value of a+c is at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100 etc.). The value of b is at least 7 (e.g. at least 8, 9, 10, 11, 12, 13, 14, 15, 16, 17, 18, 19, 20, 21, 22, 23, 24, 25, 30, 35, 40, 45, 50, 60, 70, 80, 90, 100 etc.). It is preferred that the value of a+b+c is at least 9 (e.g. at least 10, 11, 12, 13, 14, 15, 16, 17, 18, 19, 20, 21, 22, 23, 24, 25, 30, 35, 40, 45, 50, 60, 70, 80, 90, 100 etc.). It is preferred that the value of a+b+c is at most 500 (e.g. at most 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9).

The amino acid sequence of -A- typically shares less than n % sequence identity to the a amino acids which are N-terminal of sequence -B- in SEQ IDs 7, 8, 9, 10, 11, 12, 28, 29, 30, 31, 34, 35, 36, 39, 41, 43, 67, 68 and 69 and the amino acid sequence of -C- typically shares less than n % sequence identity to the c amino acids which are C-terminal of sequence -B- in SEQ IDs 7, 8, 9, 10, 11, 12, 28, 29, 30, 31, 34, 35, 36, 39, 41 43, 67, 68 and 69. The value of n is generally 60 or less (e.g. 50, 40, 30, 20, 10 or less).

The fragment of (b) or -B- may comprise a T-cell or, preferably, a B-cell epitope of SEQ IDs 7, 8, 9, 10, 11, 12, 28, 29, 30, 31, 34, 35, 36, 39, 41 43, 67, 68 and 69. T- and B-cell epitopes can be identified empirically (e.g. using the PEPSCAN method {24, 25} or similar methods), or they can be predicted (e.g. using the Jameson-Wolf antigenic index {26}, matrix-based approaches {27}, TEPITOPE {28}, neural networks {29}, OptiMer & EpiMer {30, 31}, ADEPT {32}, Tsites {33}, hydrophilicity {34}, antigenic index {35} or the methods disclosed in reference 36 etc.). These methods have proved successful in identifying B-cell and T-cell epitopes for HIV tat and HTLV tax {e.g. 31, 37, 38, 39, 40, 41, 42, 43, 44, etc.}.

Preferred fragments of (b) or -B- are located downstream of the splice site i.e. within exon 3. Examples of such fragments are 61 to 68 (or sub-fragments thereof). A polypeptide may include one or more of these sequences. For instance, it may include two or more (e.g. 2, 3, 4) of SEQ IDs 62 to 65, preferably in that order (e.g. NH₂—O¹-62-O²-63-O³-64-O⁴-65-O⁵—COOH, where O¹ to O⁵ are optional sequences of one or more amino acids), and optionally SEQ ID 61 as well (preferably upstream of SEQ ID 62). Other polypeptides may include SEQ ID 66 and/or SEQ ID 67.

Thus the invention provides a polypeptide comprising: (a) an amino acid sequence selected from the group consisting of SEQ IDs 61 to 68; (b) a fragment of at least x amino acids of (a); or (c) a polypeptide sequence having at least s % identity to (a).

The invention also provides a polypeptide comprising: (a) an amino acid sequence selected from the group consisting of SEQ IDs 78 to 277; (b) a fragment of at least x amino acids of (a); or (c) a polypeptide sequence having at least s % identity to (a).

Within the group consisting of SEQ IDs 7, 8, 9, 10, 11, 12, 28, 29, 30, 31, 34, 35, 36, 39, 41, 43, 67, 68 and 69, a preferred subset is SEQ IDs 7, 8, 11 and 12 (PCAP2, PCAP3, PCAP4 and PCAP4a).

Preferred polypeptides may bind to RNA comprising SEQ ID 49.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of reference 20. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is taught in reference 45.

Polypeptides of the invention can be prepared in many ways e.g. by chemical synthesis (at least in part), by digesting longer polypeptides using proteases, by translation from RNA, by purification from cell culture (e.g. from recombinant expression), from the organism itself (e.g. isolation from prostate or breast tissue), from a cell line source etc.

Polypeptides of the invention can be prepared in various forms (e.g. native, fusions, glycosylated, non-glycosylated etc.).

Polypeptides of the invention may be attached to a solid support.

Polypeptides of the invention may comprise a detectable label (e.g. a radioactive or fluorescent label, or a biotin label).

In general, the polypeptides of the subject invention are provided in a non-naturally occurring environment e.g. they are separated from their naturally-occurring environment. In certain embodiments, the subject polypeptide is present in a composition that is enriched for the polypeptide as compared to a control. As such, purified polypeptide is provided, whereby purified is meant that the polypeptide is present in a composition that is substantially free of other expressed polypeptides, where by substantially free is meant that less than 90%, usually less than 60% and more usually less than 50% of the composition is made up of other expressed polypeptides.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the polypeptide (e.g. a functional domain and/or, where the polypeptide is a member of a polypeptide family, a region associated with a consensus sequence). Selection of amino acid alterations for production of variants can be based upon the accessibility (interior vs. exterior) of the amino acid (e.g. ref. 46), the thermostability of the variant polypeptide (e.g. ref. 47), desired glycosylation sites (e.g. ref. 48), desired disulfide bridges (e.g. refs. 49 & 50), desired metal binding sites (e.g. refs. 51 & 52), and desired substitutions with in proline loops (e.g. ref. 53). Cysteine-depleted muteins can be produced as disclosed in reference 54.

### C.4—Antibody Materials

The invention also provides isolated antibodies, or antigen-binding fragments thereof, that bind to a polypeptide of the invention. The invention also provides isolated antibodies or antigen binding fragments thereof, that bind to a polypeptide encoded by a polynucleotide of the invention.

Antibodies of the invention may be polyclonal or monoclonal and may be produced by any suitable means (e.g. by recombinant expression).

Antibodies of the invention may include a label. The label may be detectable directly, such as a radioactive or fluorescent label. Alternatively, the label may be detectable indirectly, such as an enzyme whose products are detectable (e.g. luciferase, β-galactosidase, peroxidase etc.).

Antibodies of the invention may be attached to a solid support.

Antibodies of the invention may be prepared by administering (e.g. injecting) a polypeptide of the invention to an appropriate animal (e.g. a rabbit, hamster, mouse or other rodent).

Antigen-binding fragments of antibodies include Fv, scFv, Fc, Fab, F(ab′)₂ etc.

To increase compatibility with the human immune system, the antibodies may be chimeric or humanized {e.g. refs. 55 & 56}, or fully human antibodies may be used. Because humanized antibodies are far less immunogenic in humans than the original non-human monoclonal antibodies, they can be used for the treatment of humans with far less risk of anaphylaxis. Thus, these antibodies may be preferred in therapeutic applications that involve in vivo administration to a human such as, use as radiation sensitizers for the treatment of neoplastic disease or use in methods to reduce the side effects of cancer therapy.

Humanized antibodies may be achieved by a variety of methods including, for example: (1) grafting non-human complementarity determining regions (CDRs) onto a human framework and constant region (“humanizing”), with the optional transfer of one or more framework residues from the non-human antibody; (2) transplanting entire non-human variable domains, but “cloaking” them with a human-like surface by replacement of surface residues (“veneering”). In the present invention, humanized antibodies will include both “humanized” and “veneered” antibodies. {57, 58, 59, 60, 61, 62, 63}.

CDRs are amino acid sequences which together define the binding affinity and specificity of a Fv region of a native immunoglobulin binding site {e.g. refs. 64 & 65}.

The phrase “constant region” refers to the portion of the antibody molecule that confers effector functions. In chimeric antibodies, mouse constant regions are substituted by human constant regions. The constant regions of humanized antibodies are derived from human immunoglobulins. The heavy chain constant region can be selected from any of the 5 isotypes: alpha, delta, epsilon, gamma or mu.

One method of humanizing antibodies comprises aligning the heavy and light chain sequences of a non-human antibody to human heavy and light chain sequences, replacing the non-human framework residues with human framework residues based on such alignment, molecular modeling of the conformation of the humanized sequence in comparison to the conformation of the non-human parent antibody, and repeated back mutation of residues in the framework region which disturb the structure of the non-human CDRs until the predicted conformation of the CDRs in the humanized sequence model closely approximates the conformation of the non-human CDRs of the parent non-human antibody. Such humanized antibodies may be further derivatized to facilitate uptake and clearance e.g, via Ashwell receptors. {refs. 66 & 67}

Humanized or fully-human antibodies can also be produced using transgenic animals that are engineered to contain human immunoglobulin loci. For example, ref. 68 discloses transgenic animals having a human Ig locus wherein the animals do not produce functional endogenous immunoglobulins due to the inactivation of endogenous heavy and light chain loci. Ref. 69 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. Ref. 70 discloses the use of the Cre/Lox system to modify the immunoglobulin locus in a mammal, such as to replace all or a portion of the constant or variable region to form a modified antibody molecule. Ref. 71 discloses non-human mammalian hosts having inactivated endogenous Ig loci and functional human Ig loci. Ref. 72 discloses methods of making transgenic mice in which the mice lack endogenous heavy claims, and express an exogenous immunoglobulin locus comprising one or more xenogeneic constant regions.

Using a transgenic animal described above, an immune response can be produced to a PCAV polypeptide, and antibody-producing cells can be removed from the animal and used to produce hybridomas that secrete human monoclonal antibodies. Immunization protocols, adjuvants, and the like are known in the art, and are used in immunization of, for example, a transgenic mouse as described in ref. 73. The monoclonal antibodies can be tested for the ability to inhibit or neutralize the biological activity or physiological effect of the corresponding polypeptide.

### D—Comparison with Control Samples

### D.1—the Control

HML-2 transcripts are up-regulated in tumors. To detect such up-regulation, a reference point is needed i.e. a control. Analysis of the control sample gives a standard level of RNA and/or protein expression against which a patient sample can be compared.

A negative control gives a background or basal level of expression against which a patient sample can be compared. Higher levels of expression product relative to a negative control, such as a lifetime baseline or pooled normal samples, indicate that the patient from whom the sample was taken has a tumor. Conversely, equivalent levels of expression product indicate that the patient does not have a HML-2-related tumor.

A positive control gives a level of expression against which a patient sample can be compared. Equivalent or higher levels of expression product relative to a positive control indicate that the patient from whom the sample was taken has a tumor. Conversely, lower levels of expression product indicate that the patient does not have a HML-2 related tumor.

For direct or indirect RNA measurement, or for direct polypeptide measurement, a negative control will generally comprise cells which are not from a tumor cell (e.g. a breast tumor or a prostate tumor). For indirect polypeptide measurement, a negative control will generally be a blood sample from a patient who does not have a tumor. The negative control could be a sample from the same patient as the patient sample, but from a tissue in which HML-2 expression is not up-regulated e.g. a non-tumor non-prostate cell for a male, or a non-tumor non-breast cell for a female. The negative control could be a prostate or breast cell from the same patient as the patient sample, but taken at an earlier stage in the patient's life. The negative control could be a cell from a patient without a tumor. This cell may or may not be a prostate/breast cell. The negative control cell could be a prostate cell from a patient with BPH. The negative control could be normal semen, seminal fluid, colostrum, breast milk, etc.

For direct or indirect RNA measurement, or for direct polypeptide measurement, a positive control will generally comprise cells from the type of tumor in question. For indirect polypeptide measurement, a negative control will generally be a blood sample from a patient who has a prostate tumor or breast tumor. The negative control could be a prostate or breast tumor cell from the same patient as the patient sample, but taken at an earlier stage in the patient's life (e.g. to monitor remission). The positive control could be a cell from another patient with a prostate or breast tumor. The positive control could be a prostate cell line or a breast cell line.

Other suitable positive and negative controls will be apparent to the skilled person.

HML-2 expression in the control can be assessed at the same time as expression in the patient sample. Alternatively, HML-2 expression in the control can be assessed separately (earlier or later).

Rather than actually compare two samples, however, the control may be an absolute control i.e. a level of expression which has been empirically determined from samples taken from tumor patients (e.g. under standard conditions).

### D.2—Degree of Up-Regulation

The up-regulation relative to the control (100%) will usually be at least 150% (e.g. 200%, 250%, 300%, 400%, 500%, 600% or more).

### D.3—Diagnosis

The invention provides a method for diagnosing cancer. It will be appreciated that “diagnosis” according to the invention can range from a definite clinical diagnosis of disease to an indication that the patient should undergo further testing which may lead to a definite diagnosis. For example, the method of the invention can be used as part of a screening process, with positive samples being subjected to further analysis.

Furthermore, diagnosis includes monitoring the progress of cancer in a patient already known to have the cancer. Cancer can also be staged by the methods of the invention.

The efficacy of a treatment regimen (therametrics) of a cancer can also monitored by the method of the invention e.g. to determine its efficacy.

Susceptibility to cancer can also be detected e.g. where up-regulation of expression has occurred, but before cancer has developed. Prognostic methods are also encompassed.

Of the various types of cancer, the invention is particularly suited to prostate cancer (including prostatic intraepithelial neoplasia) and breast cancer (including mammary carcinoma).

All of these techniques fall within the general meaning of “diagnosis” in the present invention.

### E—the Putative Tar

HIV Tat acts as a transcription factor and its RNA target is the TAR. SEQ IDs 14 and 49 are examples of 150 nucleotide RNAs comprising a putative HML-2 TAR. As for HIV, the minimal tat-binding motif in the TAR may be shorter than these two molecules.

The invention provides an isolated polynucleotide of comprising: (a) the nucleotide sequence of SEQ ID 14 or 49; (b) a fragment of at least x nucleotides of (a); (c) a nucleotide sequence having at least s % identity to (a); or (d) the complement of (a), (b) or (c).

The isolated polynucleotide is preferably shorter than 250 nucleotides (e.g. shorter than 240, 230, 220, 210, 200, 190, 180, 170, 160, or 150 nucleotides).

The value of x is at least 7 (e.g. at least 8, 9, 10, 11, 12, 13, 14, 15, 16, 17, 18, 19, 20, 21, 22, 23, 24, 25, 30, 35, 40, 45, 50, 60, 70, 75, 80, 90, 100 etc.). The value of x may be less than 2000 (e.g. less than 1000, 500, 100, or 50).

The value of s is preferably at least 50 (e.g. at least 55, 60, 65, 70, 75, 80, 85, 90, 91, 92, 93, 94, 95, 96, 97, 98, 99, 99.5, 99.9 etc.).

The isolated polynucleotide can preferably bind to a protein comprising the amino acid sequence SEQ ID 7, 8 and/or 9 (putative tat analogs).

### F—Inhibiting PCAP Function

Inhibiting the Tat/TAR interaction has been used for HIV therapy, and inhibition of Tax function has been used for HTLV therapy. By analogy, inhibiting the equivalent functions in PCAV offers ways of treating cancer, and also for treating other diseases linked to HERV-K viruses (e.g. testicular cancer {194}, multiple sclerosis {74}, insulin-dependent diabetes mellitus (IDDM) (75) etc.).

Various methods have been proposed for inhibiting the Tat/TAR interaction e.g.:

The use of RNA decoys comprising multiple TARs to sequester Tat {76}

Antisense Tat {77}

Dominant negative Tat mutants {78}

Tat Ribozymes {79}

Anti-TAR hammerhead ribozymes {80}

The use of small molecule inhibitors of the Tat/TAR interaction {81, 82, 83}

Use of aptamers {84}

Use of inhibitory RNAs (siRNAs) for RNA interference {85}

Similar approaches have been used for inhibiting Tax function {86, 87, 88}, although a significant difference between Tax and Tat is that Tat binds nucleic acid directly. All of these methods can be applied to the putative PCAV tat/TAR interaction or Tax function.

The invention therefore provides the following, together with their use as pharmaceuticals and their use in the manufacture of a medicament for treating prostate cancer, testicular cancer, multiple sclerosis and/or insulin-dependent diabetes mellitus:

A polynucleotide encoding or comprising two or more copies of the putative HML-2 TAR;

A polynucleotide complementary to a putative tat-coding sequence;

A polypeptide which can bind to a functional putative tat and act in a transdominant way;

A ribozyme which can attack tat and/or tar sequences;

Small molecule inhibitors of the putative Tat/TAR interaction;

Antibodies or oligobodies {89,90} which specifically bind to putative tat; and

Aptamer inhibitors of the putative Tat/TAR interaction.

Small inhibitory RNAs {e.g. refs. 91 to 96} complementary to the putative TAR sequence.

In relation to transdominant inhibitors of putative tat function, the invention provides a protein as defined in section C.3 above, comprising: (a) an amino acid sequence selected from the group consisting of SEQ IDs 10, 11, 12 and 13; (b) a fragment of at least x amino acids of (a); or (c) a polypeptide sequence having at least s % identity to (a). Proteins having amino acid sequences SEQ IDs 10, 11, 12 and β have all been found to suppress the activity of putative tat, with SEQ ID β (cORF) being the strongest dominant negative.

### Screening Methods and Drug Design

The invention also provides methods of screening for compounds with activity against cancer, comprising: contacting a test compound with a putative Tat polynucleotide or polypeptide, or with a putative TAR polynucleotide; and detecting a binding interaction between the test compound and the polynucleotide/polypeptide. A binding interaction indicates potential anti-cancer efficacy of the test compound.

The invention also provides methods of screening for compounds with activity against prostate cancer, comprising: contacting a test compound with a putative Tat polypeptide of the invention; and assaying the function of the polypeptide. Inhibition of the polypeptide's function (e.g. loss of expression of a reporter gene driven by the PCAV LTR, as described in the examples herein) indicates potential anti-cancer efficacy of the test compound.

Typical test compounds include, but are not restricted to peptides (including cyclic peptides {82}), peptoids, proteins, lipids, metals, nucleotides, nucleosides, small organic molecules {97}, antibiotics, polyamines, and combinations and derivatives thereof. Small organic molecules have a molecular weight of more than 50 and less than about 2,500 daltons, and most preferably between about 300 and about 800 daltons. Complex mixtures of substances, such as extracts containing natural products, or the products of mixed combinatorial syntheses, can also be tested and the component that binds to the target RNA can be purified from the mixture in a subsequent step.

Test compounds may be derived from large libraries of synthetic or natural compounds {98}. For instance, synthetic compound libraries are commercially available from Maybridge Chemical Co. (Trevillet, Cornwall, UK) or Aldrich (Milwaukee, Wis.). Alternatively, libraries of natural compounds in the form of bacterial, fungal, plant and animal extracts may be used. Additionally, test compounds may be synthetically produced using combinatorial chemistry either as individual compounds or as mixtures.

Agonists or antagonists of the polypeptides of the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of an agonist or antagonist of a HML-2 polypeptide. Such agonists and antagonists can be used to modulate, enhance, or inhibit HML-2 expression and/or function. {99}

The present invention relates to methods of using the polypeptides of the invention (e.g. recombinantly produced HML-2 polypeptides) to screen compounds for their ability to bind or otherwise modulate, such as, inhibit, the activity of HML-2 polypeptides, and thus to identify compounds that can serve, for example, as agonists or antagonists of the HML-2 polypeptides. In one screening assay, the HML-2 polypeptide is incubated with cells susceptible to the growth stimulatory activity of HML-2, in the presence and absence of a test compound. The HML-2 activity altering or binding potential of the test compound is measured. Growth of the cells is then determined. A reduction in cell growth in the test sample indicates that the test compound binds to and thereby inactivates the HML-2 polypeptide, or otherwise inhibits the HML-2 polypeptide activity.

Transgenic animals (e.g. rodents) that have been transformed to over-express HML-2 genes can be used to screen compounds in vivo for the ability to inhibit development of tumors resulting from HML-2 over-expression or to treat such tumors once developed. Transgenic animals that have prostate tumors of increased invasive or malignant potential can be used to screen compounds, including antibodies or peptides, for their ability to inhibit the effect of HML-2 polypeptides. Such animals can be produced, for example, as described in the examples herein.

Screening procedures such as those described above are useful for identifying agents for their potential use in pharmacological intervention strategies in prostate cancer treatment. Additionally, polynucleotide sequences corresponding to HML-2, including LTRs, may be used to assay for inhibitors of elevated gene expression.

Potent inhibitors of HERV-K protease are already known {100}. Inhibition of HERV-K protease by HIV-1 protease inhibitors has also been reported {101}. These compounds can be studied for use in prostate cancer therapy, and are also useful lead compounds for drug design.

Transdominant negative mutants of cORF have also been reported {102,103}. Transdominant cORF mutants can be studied for use in prostate cancer therapy.

Antisense oligonucleotides complementary to HML-2 mRNA can be used to selectively diminish or oblate the expression of the polypeptide. More specifically, antisense constructs or antisense oligonucleotides can be used to inhibit the production of HML-2 polypeptide(s) in prostate tumor cells. Antisense mRNA can be produced by transfecting into target cancer cells an expression vector with a HML-2 polynucleotide of the invention oriented in an antisense direction relative to the direction of PCAV-mRNA transcription. Appropriate vectors include viral vectors, including retroviral vectors, as well as non-viral vectors. Alternately, antisense oligonucleotides can be introduced directly into target cells to achieve the same goal. Oligonucleotides can be selected/designed to achieve the highest level of specificity and, for example, to bind to a PCAV-mRNA at the initiator ATG.

Monoclonal antibodies to HML-2 polypeptides can be used to block the action of the polypeptides and thereby control growth of cancer cells. This can be accomplished by infusion of antibodies that bind to HML-2 polypeptides and block their action.

The invention also provides high-throughput screening methods for identifying compounds that bind to a Tat and/or TAR. Preferably, all the biochemical steps for this assay are performed in a single solution in, for instance, a test tube or microtitre plate, and the test compounds are analyzed initially at a single compound concentration. for the purposes of high throughput screening, the experimental conditions are adjusted to achieve a proportion of test compounds identified as “positive” compounds from amongst the total compounds screened. The assay is preferably set to identify compounds with an appreciable affinity towards the target e.g., when 0.1% to 1% of the total test compounds from a large compound library are shown to bind to a given target with a Kᵢ of 10 μM or less (e.g. 1 μM, 100 nM, 10 nM, or less)

The invention also provides structure-based drug design techniques which can be applied to structural representations of the putative Tat and/or putative TAR in order to identify compounds that can block their putative interaction. A variety of suitable techniques {e.g. ref. 104} are available to the skilled person.

Software packages for implementing molecular modelling techniques for use in structure-based drug design include SYBYL {105}, AMBER {106}, CERIUS^{2 {107}}, INSIGHT II {107}, CATALYST {107}, QUANTA {107}, HYPERCHEM {108}, CHEMSITE {109}, etc. This software can be used to determine binding surfaces of the putative Tat and/or putative TAR in order to reveal features such as van der Waals contacts, electrostatic interactions, and/or hydrogen bonding opportunities.

The invention also provides in silico screening methods for identifying compounds that bind to putative Tat and/or TAR. Structural representations of potential ligands are saved in a computer readable format, such as SD or MDL formats. A 3D structure of the ligands is preferably generated from the 2D representation using a program such as CORINA, CONCORDE or InsightII. Once a ligand has been identified which interacts in silico with a receptor, this may be provided (synthesised, purified or purchased, for instance) and the interaction can be verified experimentally. The invention provides a ligand identified using the methods of the invention.

Structure-based in silico screening has been used to identify inhibitors of the Tat/TAR interaction of HIV {110}.

Efficacy of these various methods can be tested by monitoring expression of polynucleotides and/or polypeptides of the invention after administration of the composition of the invention. All of the methods previously successfully used in tat-based HIV immunization can be used.

### G—Vaccines

Tat protein has been used as a vaccine antigen for HIV therapy, and Tax protein has been used as a vaccine antigen for HTLV therapy. Polypeptide vaccines {111,112,113,114,115} and DNA vaccines {116,117} have both been proposed. By analogy, the polypeptides of the invention can be used for immunizing against prostate or breast cancer, and also for treating other diseases linked to HERV-K viruses (e.g. testicular cancer, multiple sclerosis, IDDM etc.).

The invention therefore provides a composition comprising (a) a polypeptide as defined in section C.3 above and (b) a pharmaceutically acceptable carrier. The invention also provides a composition comprising (a) a polynucleotide encoding a polypeptide as defined above and (b) a pharmaceutically acceptable carrier.

The composition may additionally comprise an adjuvant. For example, the composition may comprise one or more of the following adjuvants: (1) oil-in-water emulsion formulations (with or without other specific immunostimulating agents such as muramyl peptides (see below) or bacterial cell wall components), such as for example (a) MF59™ {118; Chapter 10 in ref. 119}, containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (2) saponin adjuvants, such as QS21 or Stimulon™ (Cambridge Bioscience, Worcester, Mass.) may be used or particles generated therefrom such as ISCOMs (immunostimulating complexes), which ISCOMS may be devoid of additional detergent {120}; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) {e.g. 121, 122}; (6) combinations of 3dMPL with, for example, QS21 and/or oil-in-water emulsions {e.g. 123, 124, 125}; (7) oligonucleotides comprising CpG motifs i.e. containing at least one CG dinucleotide, with 5-methylcytosine optionally being used in place of cytosine; (8) a polyoxyethylene ether or a polyoxyethylene ester {126}; (9) a polyoxyethylene sorbitan ester surfactant in combination with an octoxynol {127} or a polyoxyethylene alkyl ether or ester surfactant in combination with at least one additional non-ionic surfactant such as an octoxynol {128}; (10) an immunostimulatory oligonucleotide (e.g. a CpG oligonucleotide) and a saponin {129}; (11) an immunostimulant and a particle of metal salt {130}; (12) a saponin and an oil-in-water emulsion {131}; (13) a saponin (e.g. QS21) +3dMPL +IL-12 (optionally +a sterol) {132}; (14) aluminium salts, preferably hydroxide or phosphate, but any other suitable salt may also be used (e.g. hydroxyphosphate, oxyhydroxide, orthophosphate, sulphate etc. {chapters 8 & 9 of ref. 119}). Mixtures of different aluminium salts may also be used. The salt may take any suitable form (e.g. gel, crystalline, amorphous etc.); (15) chitosan; (16) cholera toxin or E. coli heat labile toxin, or detoxified mutants thereof {133}; (17) microparticles of poly(a-hydroxy)acids, such as PLG; (18) other substances that act as immunostimulating agents to enhance the efficacy of the composition. Aluminium salts and/or MF59™ are preferred.

The composition is preferably sterile and/or pyrogen-free. It will typically be buffered around pH 7.

The composition is preferably an immunogenic composition and is more preferably a vaccine composition. The composition can be used to raise antibodies in a mammal (e.g. a human).

Vaccines of the invention may be prophylactic (i.e. to prevent disease) or therapeutic (i.e. to reduce or eliminate the symptoms of a disease).

Efficacy can be tested by monitoring expression of polynucleotides and/or polypeptides of the invention after administration of the composition of the invention. All of the methods previously used in tat-based HIV immunization can be used.

### H—Pharmaceutical Compositions

The invention provides a pharmaceutical composition comprising polynucleotide, polypeptide, or antibody as defined above. The invention also provides their use as medicaments, and their use in the manufacture of medicaments for treating cancer. The invention also provides a method for raising an immune response, comprising administering an immunogenic dose of polynucleotide or polypeptide of the invention to an animal.

Pharmaceutical compositions encompassed by the present invention include as active agent, the polynucleotides, polypeptides, or antibodies of the invention disclosed herein in a therapeutically effective amount. An “effective amount” is an amount sufficient to effect beneficial or desired results, including clinical results. An effective amount can be administered in one or more administrations. For purposes of this invention, an effective amount is an amount that is sufficient to palliate, ameliorate, stabilize, reverse, slow or delay the symptoms and/or progression of cancer.

The compositions can be used to treat cancer as well as metastases of primary cancer. In addition, the pharmaceutical compositions can be used in conjunction with conventional methods of cancer treatment, e.g. to sensitize tumors to radiation or conventional chemotherapy. The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e. arresting its development; or (c) relieving the disease symptom, i.e. causing regression of the disease or symptom.

Where the pharmaceutical composition comprises an antibody that specifically binds to a gene product encoded by a differentially expressed polynucleotide, the antibody can be coupled to a drug for delivery to a treatment site or coupled to a detectable label to facilitate imaging of a site comprising cancer cells, such as prostate cancer cells. Methods for coupling antibodies to drugs and detectable labels are well known in the art, as are methods for imaging using detectable labels.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/kg to about 50 mg/kg or: about 0.05 mg/kg to about 10 mg/kg of the compositions of the present invention in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g. mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins, or reference 134.

Once formulated, the compositions contemplated by the invention can be (1) administered directly to the subject (e.g. as polynucleotide, polypeptides, small molecule agonists or antagonists, and the like); or (2) delivered ex vivo, to cells derived from the subject (e.g. as in ex vivo gene therapy). Direct delivery of the compositions will generally be accomplished by parenteral injection, e.g. subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art {e.g. ref. 135}. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Differential expression PCAV polynucleotides has been found to correlate with tumors. The tumor can be amenable to treatment by administration of a therapeutic agent based on the provided polynucleotide, corresponding polypeptide or other corresponding molecule (e.g. antisense, ribozyme, etc.). In other embodiments, the disorder can be amenable to treatment by administration of a small molecule drug that, for example, serves as an inhibitor (antagonist) of the function of the encoded gene product of a gene having increased expression in cancerous cells relative to normal cells or as an agonist for gene products that are decreased in expression in cancerous cells (e.g. to promote the activity of gene products that act as tumor suppressors).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents includes local or systemic administration, including injection, oral administration, particle gun or catheterized administration, and topical administration. Preferably, the therapeutic polynucleotide composition contains an expression construct comprising a promoter operably linked to a polynucleotide of the invention. Various methods can be used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. An antisense composition is directly administered to the surface of the tumor, for example, by topical application of the composition. X-ray imaging is used to assist in certain of the above delivery methods.

Targeted delivery of therapeutic compositions containing an antisense polynucleotide, subgenomic polynucleotides, or antibodies to specific tissues can also be used. Receptor-mediated DNA delivery techniques are described in, for example, references 136 to 141. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g. for enhancing or inhibiting levels of the encoded gene product) and efficacy of transformation and expression are considerations which will affect the dosage required for ultimate efficacy of the antisense subgenomic polynucleotides. Where greater expression is desired over a larger area of tissue, larger amounts of antisense subgenomic polynucleotides or the same amounts re-administered in a successive protocol of administrations, or several administrations to different adjacent or close tissue portions of, e.g., a tumor site, may be required to effect a positive therapeutic outcome. In all cases, routine experimentation in clinical trials will determine specific ranges for optimal therapeutic effect.

The therapeutic polynucleotides and polypeptides of the present invention can be delivered using gene delivery vehicles. The gene delivery vehicle can be of viral or non-viral origin (see generally references 142, 143, 144 and 145). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (e.g. references 146 to 156), alphavirus-based vectors (e.g. Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), adenovirus vectors and adeno-associated virus (AAV) vectors (e.g. see refs. 157 to 162). Administration of DNA linked to killed adenovirus {163} can also be employed.

Non-viral delivery vehicles and methods can also be employed, including, but not limited to, polycationic condensed DNA linked or unlinked to killed adenovirus alone {e.g. 163}, ligand-linked DNA {164}, eukaryotic cell delivery vehicles cells {e.g. refs. 165 to 169} and nucleic charge neutralization or fusion with cell membranes. Naked DNA can also be employed. Exemplary naked DNA introduction methods are described in refs. 170 and 171. Liposomes that can act as gene delivery vehicles are described in refs. 172 to 176. Additional approaches are described in refs. 177 & 178.

Further non-viral delivery suitable for use includes mechanical delivery systems such as the approach described in ref. 178. Moreover, the coding sequence and the product of expression of such can be delivered through deposition of photopolymerized hydrogel materials or use of ionizing radiation {e.g. refs. 179 & 180}. Other conventional methods for gene delivery that can be used for delivery of the coding sequence include, for example, use of hand-held gene transfer particle gun or use of ionizing radiation for activating transferred gene {179 & 182}.

### I—The HML-2 Family of Human Endogenous Retroviruses

Genomes of all eukaryotes contain multiple copies of sequences related to infectious retroviruses. These endogenous retroviruses have been well studied in mice where both true infectious forms and thousands of defective retrovirus-like elements (e.g. the IAP and Etn sequence families) exist. Some members of the IAP and Etn families are “active” retrotransposons since insertions of these elements have been documented which cause germ line mutations or oncogenic transformation.

Endogenous retroviruses were identified in human genomic DNA by their homology to retroviruses of other vertebrates {183, 184}. It is believed that the human genome probably contains numerous copies of endogenous proviral DNAs, but little is known about their function. Most HERV families have relatively few members (1-50) but one family (HERV-H) consists of ˜1000 copies per haploid genome distributed on all chromosomes. The large numbers and general transcriptional activity of HERVs in embryonic and tumor cell lines suggest that they could act as disease-causing insertional mutagens or affect adjacent gene expression in a neutral or beneficial way.

The K family of human endogenous retroviruses (HERV-K) is well known {185}. It is related to the mouse mammary tumor virus (MMTV) and is present in the genomes of humans, apes and old world monkeys, but several human HERV-K proviruses are unique to humans {186}. The HERV-K family is present at 30-50 full-length copies per haploid human genome and possesses long open reading frames that potentially are translated into viral proteins {187, 188}. Two types of proviral genomes are known, which differ by the presence (type 2) or absence (type 1) of a stretch of 292 nucleotides in the overlapping boundary of the pol and env genes {189}. Some members of the HERV-K family are known to code for the gag protein and retroviral particles, which are both detectable in germ cell tumors and derived cell lines {190}. Analysis of the RNA expression pattern of full-length HERV-K has also identified a doubly-spliced RNA that encodes a 105 amino acid protein termed central ORF (‘cORF’) which is a sequence-specific nuclear RNA export factor that is functionally equivalent to the Rev protein of HIV {191}. HERV-K10 has been shown to encode a full-length gag homologous 73 kDa protein and a functional protease {192}.

Patients suffering from germ cell tumors show high antibody titers against HERV-K gag and env proteins at the time of tumor detection {193}. In normal testis and testicular tumors the HERV-K transmembrane envelope protein has been detected both in germ cells and tumor cells, but not in the surrounding tissue. In the case of testicular tumor, correlations between the expression of the env-specific mRNA, the presence of the transmembrane env, cORF and gag proteins and antibodies against HERV-K specific peptides in the serum of the patients, have been reported. Reference 194 reports that HERV-K10 gag and/or env proteins are synthesized in seminoma cells and that patients with those tumors exhibit relatively high antibody titers against gag and/or env.

Gag proteins released in form of particles from HERV-K have been identified in the cell culture supernatant of the teratocarcinoma derived cell line Tera 1. These retrovirus-like particles (termed “human teratocarcinoma derived virus” or HTDV) have been shown to have a 90% sequence homology to the HERV-K10 genome {190, 195}.

While the HERV-K family is present in the genome of every human cell, high level expression of mRNAs, proteins and particles is observed only in human teratocarcinoma cell lines {196}. In other tissues and cell lines, only a basal level of expression of mRNA has been demonstrated even using very sensitive methods {197}. The expression of retroviral proviruses is generally regulated by elements of the 5′ long terminal repeat (LTR). The activity of HERV-K LTRs is known to be up-regulated by transcriptional factors. Furthermore, the activation of expression of an endogenous retrovirus may trigger the expression of a downstream gene that triggers a neoplastic effect.

The sequence of HERV-K(II), which locates to chromosome 3, has been disclosed {198}.

HML-2 is a subgroup of the HERV-K family {199}. HERV isolates which are members of the HML-2 subgroup include HERV-K10 {189,194}, the 27 HML-2 viruses shown in FIG. 4 of reference 200, HERV-K(C7) {201}, HERV-K(II) {198}, and HERV-K(CH).

Because HML-2 is a well-recognized family, the skilled person will be able to determine without difficulty whether any particular endogenous retroviruses is or is not a HML-2. Preferred members of the HML-2 family for use in accordance with the present invention are those whose proviral genome has an LTR which has at least 75% sequence identity to SEQ ID 44 (the LTR sequence from HML-2.HOM {7}). Example LTRs include SEQ IDs 45-48.

### Disclaimers

In some embodiments, the invention may not encompass polypeptides having one of amino acid sequences SEQ IDs 69 to 76, or polypeptides comprising SEQ IDs 69 to 76 {204}.

In some embodiments, the invention may not encompass: (i) nucleic acid comprising a nucleotide sequence disclosed in reference 1; (ii) nucleic acid comprising a nucleotide sequence within SEQ IDs 1 to 225 in reference 1; (iii) a known nucleic acid; (iv) a polypeptide comprising an amino acid sequence disclosed in reference 1; (v) a polypeptide comprising an amino acid sequence within SEQ IDs 1 to 225 in reference 1; (vi) a known polypeptide; (vii) a nucleic acid or polypeptide known as of 7 Dec. 2001 (e.g. whose sequence is available in a public database such as GenBank or GeneSeq before 7 Dec. 2001); or (viii) a polypeptide or nucleic acid known as of 10 Jun. 2002 (e.g. whose sequence is available in a public database such as GenBank or GeneSeq before 10 Jun. 2002).

### DEFINITIONS

The term “comprising” means “including” as well as “consisting” e.g. a composition “comprising” X may consist exclusively of X or may include something additional e.g. X +Y.

The term “about” in relation to a numerical value x means, for example, x±10%.

The terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e. de-regulated cell division). Neoplastic cells can be malignant or benign and include tissue derived from prostate or breast cancer.

The word “substantially” does not exclude “completely” e.g. a composition which is “substantially free” from Y may be completely free from Y. Where necessary, the word “substantially” may be omitted from the definition of the invention.

### BRIEF DESCRIPTION OF DRAWINGS

FIG. 1 is a schematic representation of a human endogenous retrovirus with a depiction of the HERV-K(CH) polynucleotides and their position relative to the retrovirus.

FIG. 2 is a schematic representation of open reading frames within the HERV-K(HML-2.HOM) (also known as ‘ERVK6’) genome {7}.

FIG. 3 shows splicing events described in the prior art for HERV-K mRNAs.

FIG. 4 shows multiple splice sites identified near the 5′ and 3′ ends of the env ORF. The three reading frames are shaded differently. Five multiple-spliced products are shown beneath env ORF, and these are also shown in FIG. 5 together with a gel showing PCR products resulting from the primers shown as arrows at the top of FIG. 4.

FIG. 6 shows the adenovirus vector used in an expression assay to test for tat activity, and FIG. 7 shows the results of GFP expression driven from this vector.

FIG. 8 shows the vector used to test the activity of PCAP polypeptides, and FIG. 9 shows FACS data obtained using this vector in combination with the FIG. 6 vector.

FIG. 10 shows deletions made in the LTR of PCA-mRNA, and FIG. 11 shows GFP expression driven from these LTRs.

FIG. 12 shows data on RNA mapping of the 5′ end of PCA-mRNA.

FIG. 13 shows a predicted secondary structure for SEQ ID 14.

FIG. 14 shows northern blot analysis of PCAV transcripts in cancer cell lines. The top arrow on the left shows the position of the genomic mRNA transcript. The next arrow shows the position of the env transcript. The bottom two arrows show the positions of other ORFs. The lanes contain RNA from the following cell lines: (1) Tera 1; (2) DU145; (3) PC3; (4) MDA Pca-2b; (5) LnCaP. Tera 1 is a teratocarcinoma cell line; the others are prostatic carcinoma cell lines.

FIG. 15 illustrates the PCR strategy used to detect splice events between the LTRs, and FIG. 16 shows the results of this strategy. In FIG. 15, the horizontal line represents the HML-2 genome, the vertical lines above the genome are splice sites, and the vertical lines below the genome are ATG codons for gag, pol and env. The approximate positions of forward (F) and reverse (R) PCR primers are also shown.

FIG. 17 shows patterns of splicing in HML-2, and FIG. 18 shows the diversity of splice junctions in exon 2. FIGS. 19 to 22 show alignments of: (19) exon 1 in the splice junction region; (20) exon 1.5; (21) exon 2; and (22) exon 3. The numbers in all alignments refer to the positions in GenBank entry Y17832 of a prototype HERV-K sequence.

FIG. 23 shows the results of a RT-PCR scanning assay used to map the 5′ end of PCAV mRNAs.

FIG. 24 gives details of a RNase protection assay. Two antisense probes were used—a long probe (24B) and a short probe (24C). Both probes protected the region shown in 24A. In 24B, the position of the band expected based on the ‘usual’ 5′ end based on the position of the TATA signal is shown, plus the actual band achieved. The three lanes in 24B are: (1) Tera1; (2) no RNA; (3) probe, no RNase. The two lanes in 24C are: (1) Tera1; (2) probe, no RNase.

FIG. 25 shows the regions deleted with FIG. 13 for testing the 5′ region of mRNAs, and

FIG. 26 shows FACS analysis of GFP expression driven from the deletion mutants.

FIG. 27 shows that PCAP4 activates the HERV-K LTR (‘LTR62’) but not the murine leukemia virus LTR (‘MoLTR’). Similarly, FIG. 28 shows that PCAP4 can activate the HIV LTR, FIG. 29A shows that activates the EF1A promoter, and FIG. 29B shows it does not activate the CMV promoter.

FIG. 30 shows the subcellular localization of PCAP2.

FIG. 31 shows various cells plated in matrigel, and FIG. 32 shows cells cultured in soft agar.

FIG. 33 shows a RT-PCR analysis of various cells {204}. Lane 1 contains 200, 300, 400 and 500 bp markers. For the other lanes, even numbers lanes were obtained with RT and odd numbers were obtained without RT: (2 & 3) primary human lymphocytes; (4 & 5) transformed B cells; (6 & 7) Tera1 cells; (8 & 9) mammary carcinoma biopsy; (10 & 11) seminoma biopsy; (12 & 13) control.

FIG. 34 shows the subcellular localization of PCAP4.

FIG. 35 shows cells stained with methylene blue after three weeks of culture.

FIG. 36 shows the empty pCEP4 vector, and FIG. 37 shows NIH3T3 cells after growth for 4 days following transformation with various vectors. Cell density is given in the graph, and the cells themselves are shown at both 1× and 200× magnification.

FIG. 38 shows TUNEL analysis of cells expressing (A) PCAP2, (B) PCAP3 or (C) uninfected. In (A) and (B), the multiplicity of infection is 100, 50 and 25 from left to right.

FIG. 39 shows PCAP2-transfected PrECs (39B) as well as control PrECs (39A & C). Asterisks show cells with more than one nucleus.

FIG. 40 shows bromo-deoxyuridine labeling of PrECs for detecting cell growth.

FIG. 41 shows RT-PCR of (41A) cancerous and (41B) normal breast tissue. The positions of PCAP2 and gusB (β glucuronidase) transcripts are shown.

FIG. 42 shows immunofluorescence experiments using an anti-gag monoclonal antibody 5G2 to stain sections of tissue taken from a prostate cancer patient. FIG. 42A shows a normal prostate gland, 42B shows atrophied tissue, 42C shows a Gleason grade 3 cancer, and 42D shows a Gleason grade 4 cancer.

FIG. 43 is a FACS spectrum showing GFP expression from the MDALTR. The three traces from left to right are: (1) uninfected cells; (2) PCAP3; (3) PCAP4.

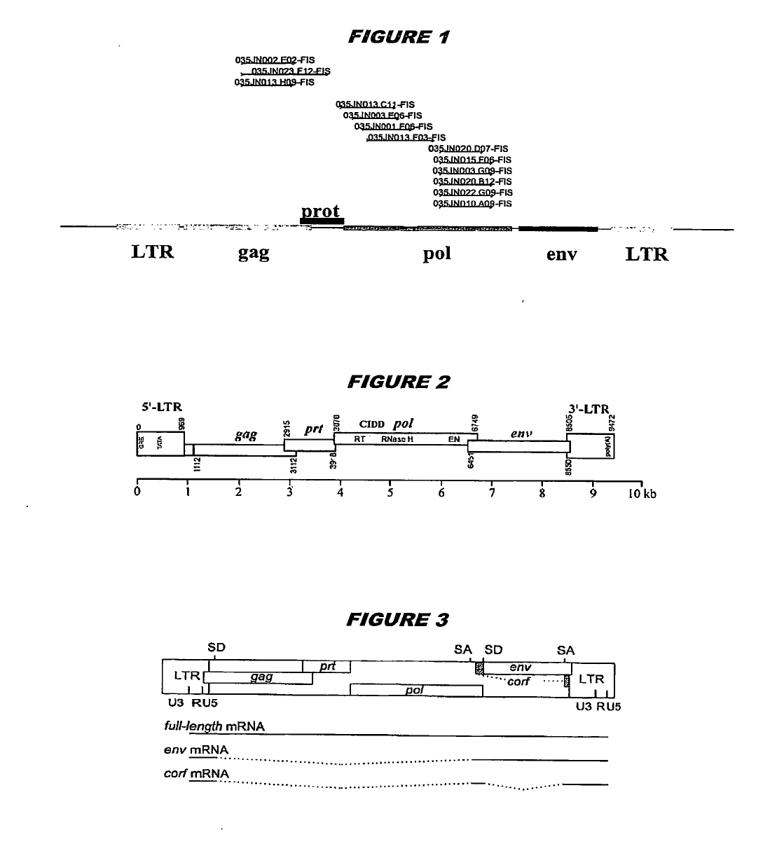

### MODES FOR CARRYING OUT THE INVENTION

Certain aspects of the present invention are described in greater detail in the non-limiting examples that follow. The examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Prostate-Associated Expression of HML-2 Sequences

Reference 1 describes the association of prostate cancer with the up-regulation of expression of the HML-2 subgroups of the HERV-K endogenous retroviruses.

### Splicing Patterns of mRNA

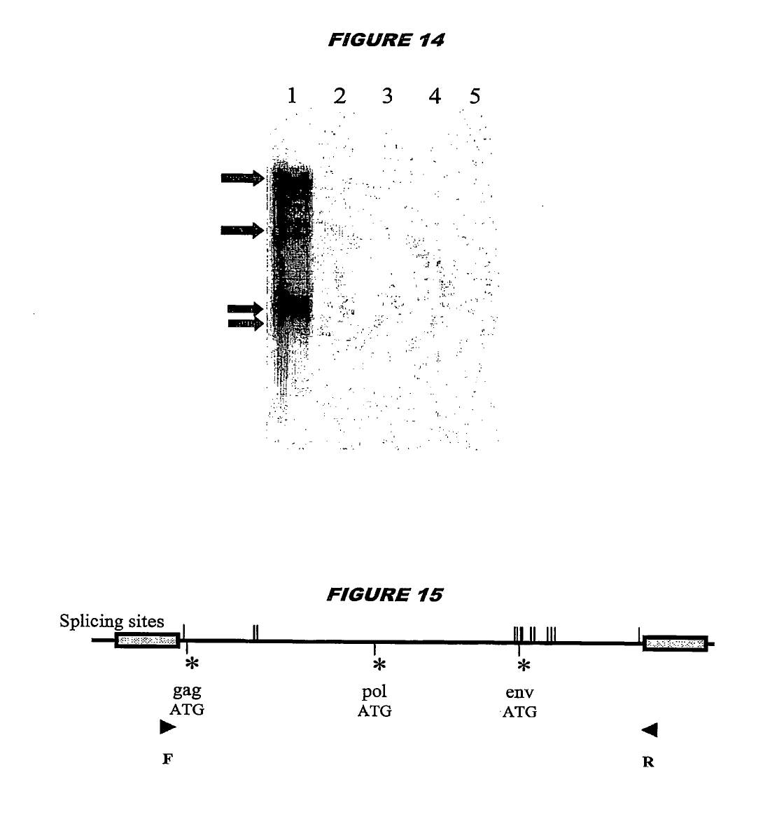

Northern blotting of prostate cancer cell lines indicates that they express PCAV transcripts of several sizes, corresponding to both full-length viral genomic sequences and to sub-genomic spliced transcripts (FIG. 14). Expression of such transcripts have also been observed in teratocarcinoma cell lines {4}, as shown in lane 1 of FIG. 14. To further characterize the splicing patterns of PCAV, a RT-PCR strategy was used that can detect any splice event between the flanking LTRs of the integrated proviral sequences. The approximate position of the forward primer (SEQ ID 15) and reverse primers (SEQ IDs 16 and 17) used in this approach, relative to the general features of the integrated PCAV genome, are indicated by arrows in FIG. 15.

DNA fragments corresponding to the transcripts of env and other ORFs could be detected in these experiments only when reverse-transcriptase was included in the RT-PCR reactions (lane 1, 3, 5, 7 and 9 in FIG. 16), indicating the they are derived from spliced PCAV mRNAs. These spliced mRNA were detected in the teratocarcinoma cell line Tera 1 (FIG. 16, lane 1) and in prostate cancer cell lines DU145, PC3, LNCaP and MDA-PCa-2b (FIG. 16). Similar results were also observed in several tumor samples obtained from prostate cancer patients.

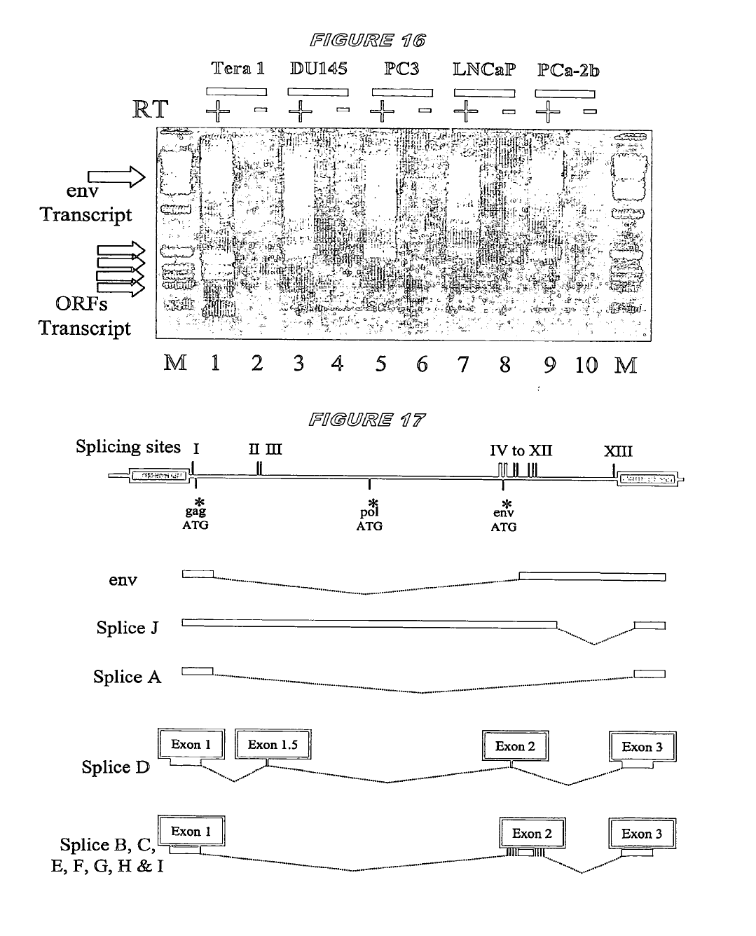

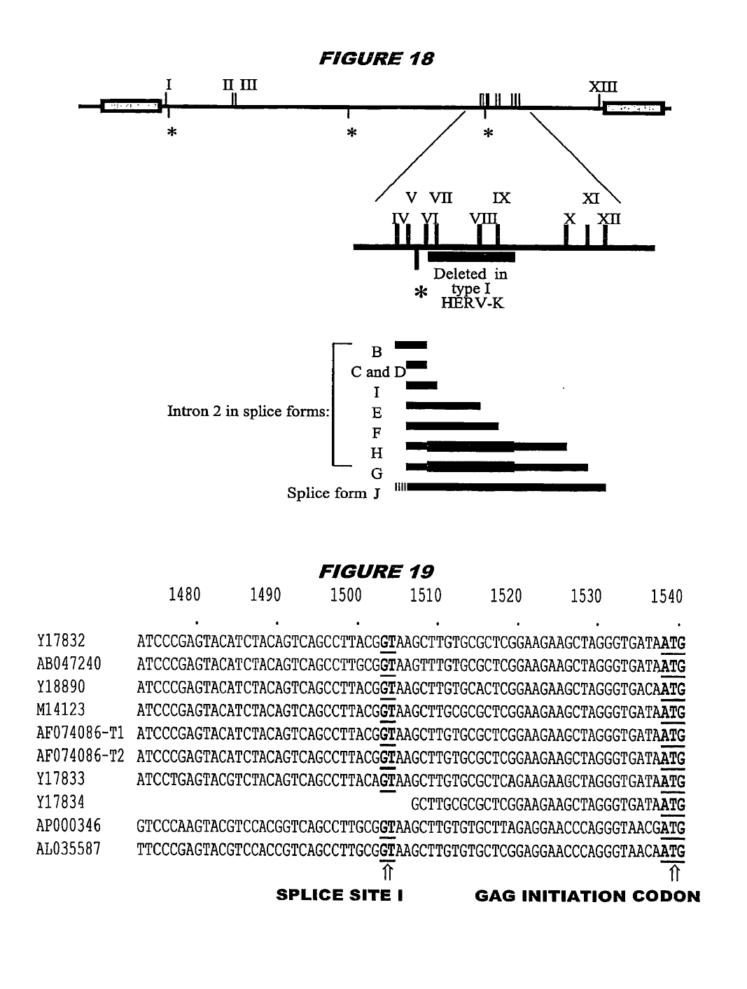

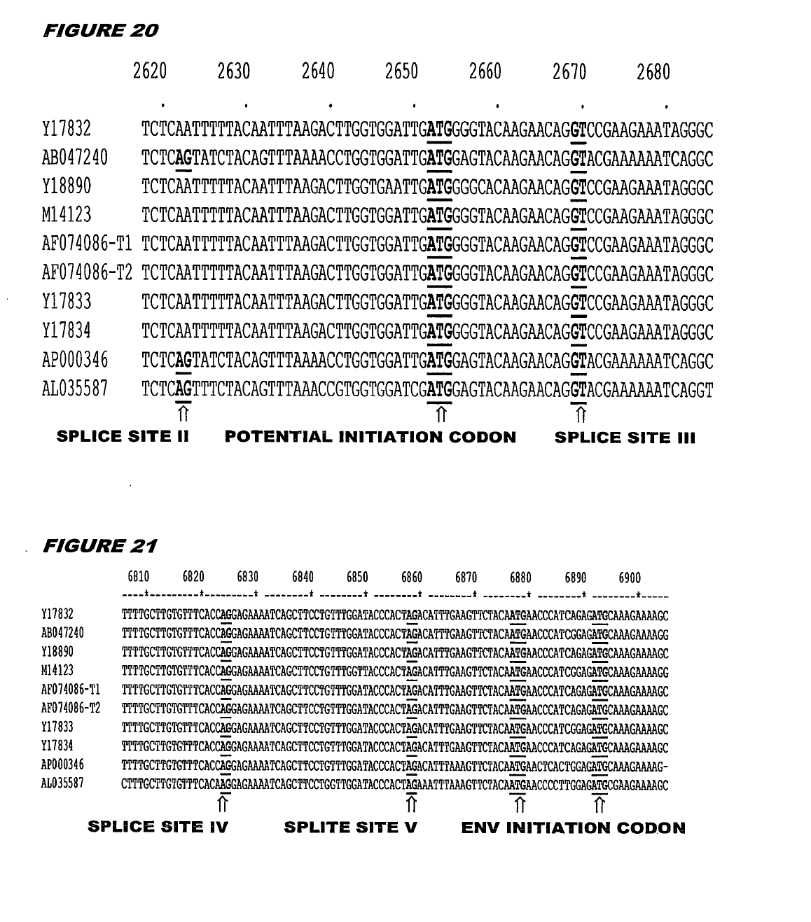

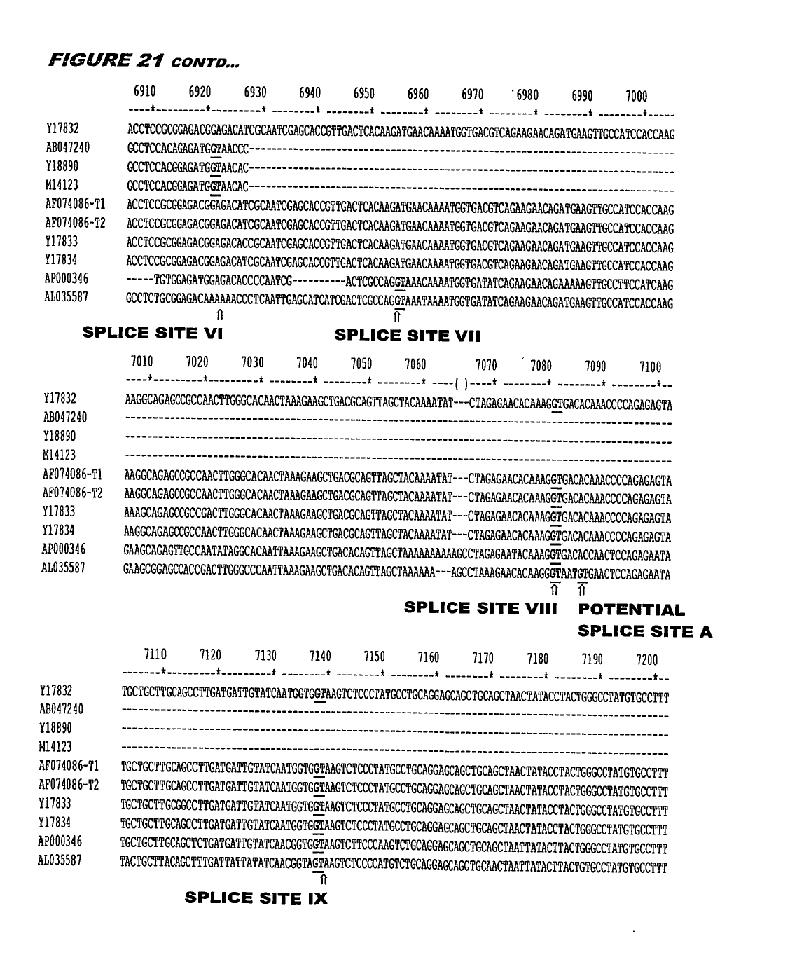

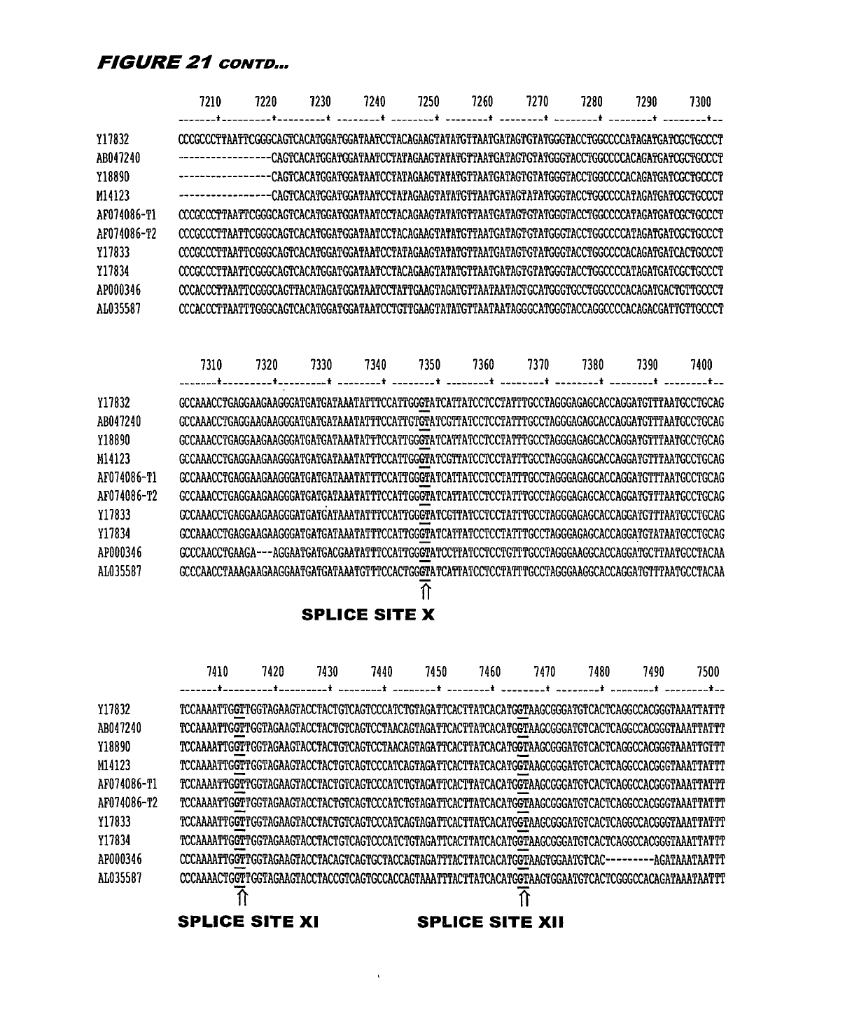

To determine the precise splicing patterns, the RT-PCR products obtained from cell lines and patient tissues were cloned and sequenced. In addition to env spliced mRNA, many other splice variants were seen (named Splice A to J in FIGS. 17 & 18; see below for consensus sequences of these splice variants). Detailed analysis of these results identify four exons (Exon 1, 1.5, 2 & 3) and β splice junctions (named I to XIII in FIGS. 17 to 22) across HML-2 genomic sequences.

Exon 1 comprises sequences from the transcription start site in the LTR to Splice Site I, as indicated schematically in FIG. 17. Splice junction I is conserved among all the integrated copies of HML-2 and is located up-stream of the initiation methionine for gag (FIG. 19, between nucleotides 1502 and 1503). All spliced mRNAs examined are precisely spliced at this site, with the exception of the Splice J mRNA, which appears to have removed only the intron between exon 2 and 3 (FIG. 17).

Exon 1.5 is very small and was only detected in the Splice D mRNA (FIG. 17). This exon is located in the gag coding sequences (between nucleotides 2624 and 2668—see FIG. 20) and encodes for a potential initiation methionine. Only some of the integrated copies of the PCAV genome contain the AG 3′ splice junction consensus sequence found at position 2622-2623 of the prototype Y17832 genome. Probably this represents either a gain or a loss of this intron in some PCAV variants during the evolution as free virus or during primate evolution as integrated viral genomes.

Exon 2 is very heterogeneous, containing two different 3′ splice junctions at the 5′ end of the exon (Splice Sites IV and V—see FIGS. 18 and 21) and seven 5′ splice junctions at the 3′ end of the exon (Splice Sites VI to XII—see FIGS. 19 and 21). In addition, Exon 2 contains other potential splice sites that were not detected in the experimental analysis. One of these potential sites, indicated in FIG. 21 as “Potential Splice Site A” may be used to generate a mRNA that encodes for an equivalent of HIV tat or HTLV tax (see below).

The size of Exon 2 in each splice variant depends on which splice sites are used in each independent splice event. FIG. 18 summarizes all of the observed splice variants (see also SEQ IDs 18-43), but in principle any other combination is possible with the potential exclusion of exons that are too large for internal exons in mRNA with three or more exons. Adding to the level of complexity, four of these Splice Sites are specific for two PCAV sequence variants found integrated in the human genome. Type I viruses (sequences AB047240, Y18890 and M14123 in FIG. 21) contain a deletion of around 300 nucleotides in the N-terminal region of env, about 35-43 nucleotides after the two potential initiation methionine (see FIG. 18). Splice Site VI is found only in Type I viruses in close proximity to the site of this deletion (see FIG. 21). Splice Sites VII, VIII and IX are only found in the Type II viruses (sequences Y17832, AF074086-T1, AF074086-T2, Y17833, Y17834, AP000346 and AL035587 in FIG. 21), as the sequence that contains them is deleted in the Type I virus. Thus, the size of the peptide encoded by exon 2 depends on both the pattern of splicing and on the type of virus from which the mRNA is derived. The initiation methionine for env is present in all detected forms of exon 2 and the open reading frame is open through all the splice sites characterized.

Exon 3 sequences begin about 90 nucleotides before the second LTR (at position 8817 of the prototype sequence Y17832, see FIG. 22) and continues into the polyadenylation sites contained within the LTR. All of the splice forms detected use the Splice Site XIII between position 8816 and 8817 (FIG. 22). A second potential splice site consensus site is located between position 8824 and 8825 (FIG. 22, Potential Splice Site B) but was not observed in any of the cDNA clones analyzed (i.e. it may be used at a low frequency). This splice site can also be use to generate a PCAV tat or tax equivalent (see below). In this exon, all three reading frames are open. Frame 1 ends at position 8871 (FIG. 22) and adds 18 amino to the encoded polypeptide when in frame with the sequences of exon 2. This reading frame is used in the previously characterized splice form called cORF in which exon 2 at Splice Site IX is joined with exon 3 at Splice site XIII to encode a PCAV rev polypeptide. Frame 2 ends at position 8995 (FIG. 9) and adds 59 amino acids to the encoded polypeptide when in frame with exon 2. This frame encodes for the PCAV tat/tax equivalent described below. The third frame in exon 3 corresponds to the C-terminus of PCAV env and ends at position 8954 in the alignments in FIG. 22.

This pattern of splicing and potential to encode multiple products depending of which splice sites are utilized resembles in general the splicing pattern of HIV and HTLV. This suggests that PCAV belongs to the lentivirus type of retroviruses. All possible splice variants are identified as SEQ IDs 18 to 43 (consensus sequences) and are described in Table 1.

### Identification of Polypeptide Similar to Tat or Tax

A defining characteristic of lentiviruses is that they encode a polypeptide that can activate transcription from the viral LTR promoter. HIV's tat polypeptide is the best understood example of these activators. The tat gene physically overlaps the rev and env genes in HIV and is made through alternative splicing of HIV mRNA spanning the env region. Tat polypeptide binds to the 5′ end of HIV mRNA at a specific site called TAR and provides HIV-specific activation.

Full-length HERV-K mRNAs can be spliced twice—once to remove gag-prt-pol and once to remove the bulk of the env gene (FIG. 3). The polypeptide encoded by the double spliced RNA has been identified and is called ‘cORF’. This polypeptide is believed to have activity similar to HIV rev, but a tat polypeptide has not previously been identified for members of the HERV-K family. Spliced PCAV-mRNAs which encode a potential tat homolog have now been identified.

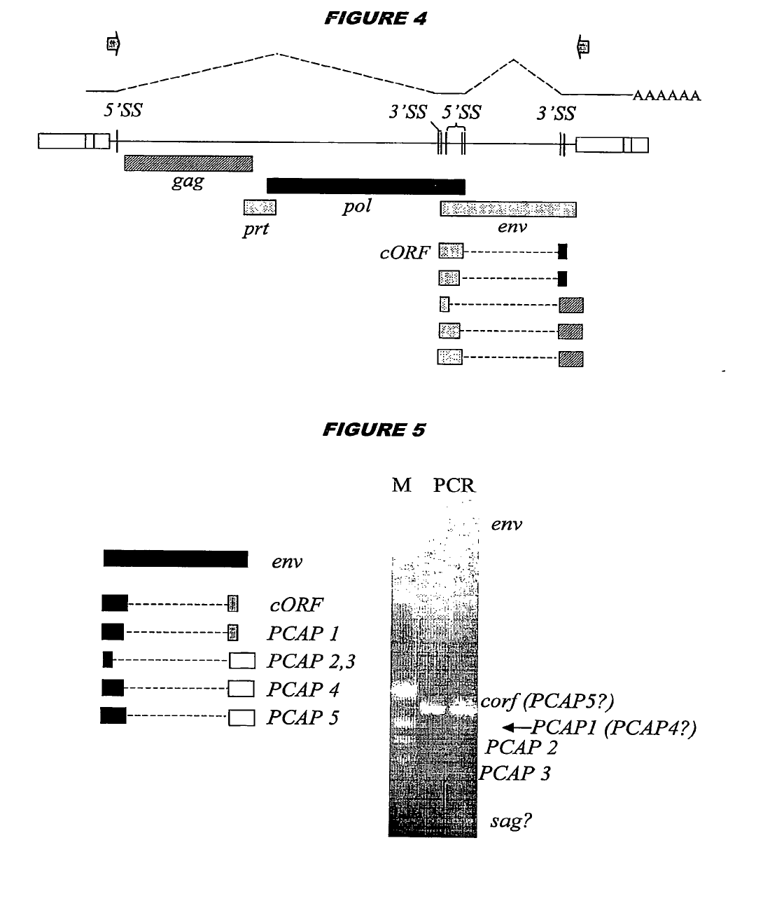

Multiple alternative splice sites in PCAV-mRNAs have been identified (FIGS. 4 and 5). These indicate that the final exon in the env region can be used in all three reading frames. Frames 1 and 2 encode env and cORF, respectively, but the third frame contains the longest open reading frame of the three. Several alternative mRNAs will connect the first coding exon to this reading frame.

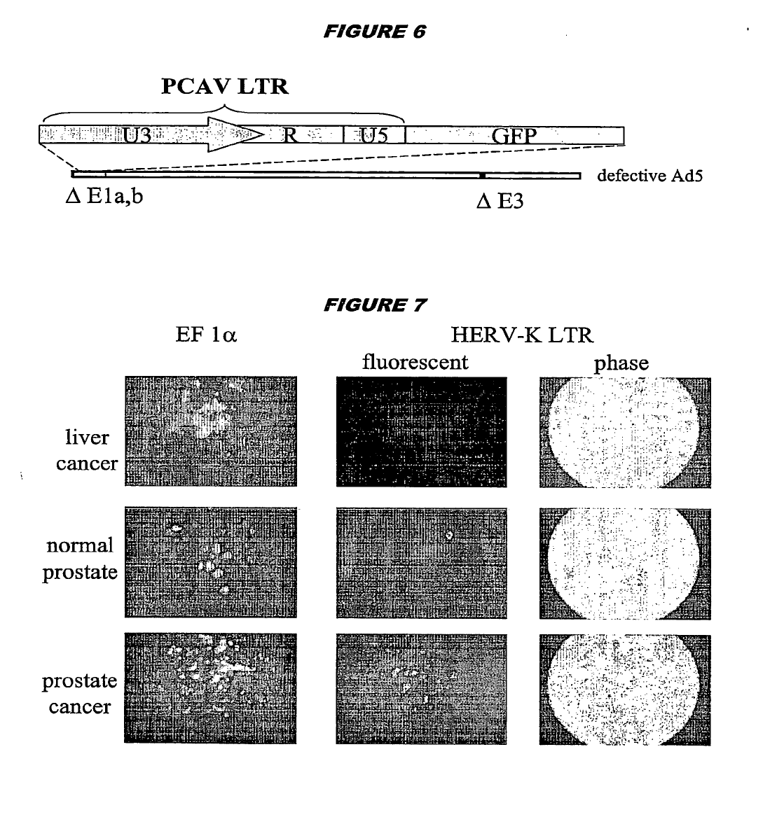

A functional expression assay was designed to determine if the third reading frame in the final env exon encodes a polypeptide with the ability to activate transcription of PCAV-mRNA. The first component of the assay is an adenovirus vector with a PCAV LTR (SEQ ID 45) driving GFP expression (FIG. 6). A variety of human cell lines were infected with this virus and fluorescence was measured either by fluorescent microscopy or FACS. As a positive control, a vector was used in which GFP expression was driven by the EFα promoter. This should be active in all eukaryotic cells.

GFP expression from this LTR was minimal in ovarian, breast, colon and liver cancer cells. It was also minimal in 293 cells, an immortalized kidney cell line, and also in primary prostate epithelium cells. GFP was easily detected in various prostate cancer cell lines (PC3, LNCaP, MDA2B PCA, DU145). Representative data are shown in FIG. 7. The GFP expression pattern exactly matches the genomics results from patient samples. These data indicate that expression driven from a PCAV-mRNA LTR is a marker for prostate cancer.

As GFP expression from the LTR appeared to be silent in primary prostate cells and active in prostate cancer, polypeptides from the env region were tested for their ability to activate expression in primary prostate cells. The coding sequences shown in FIG. 5 were inserted into expression cassettes and these were incorporated into adenovirus vectors. The first coding exon is common to env, rev and the five PCAP products. This exon contains a RNA-binding domain that also functions as a nuclear localization signal (NLS), a polypeptide dimerization region, and a highly hydrophobic sequence. The cORF polypeptide contains all three of these domains fused to a very short region in the terminal exon (FIG. 4). The PCAP1 transcript encodes a polypeptide using an alternative 5′ splice site 57 bases upstream of the normal site and deletes the hydrophobic domain from cORF. PCAP2 is derived from a type I HERV-K deletion that destroys all three domains but connects the env ATG to the third frame in the last exon. PCAP3 is similar to PCAP2, but is based on a different virus where alternative splicing instead of a deletion makes the product. PCAP4 is based on a genomic sequence where a potential 5′ splice site 52 bases upstream of the normal cORF site is connected to the 3′ splice site used in cORF, and it contains the RNA binding domain and the dimerization region fused to the 3rd coding frame in the last exon. In a separate experiment, it was found that a 3′ splice site exists 7 bases downstream of the cORF site. This site was matched to the cORF 5′ splice site and the site 57 bases upstream of this site. The product with the upstream site is called PCAP4a and has the same structure as PCAP4 but is missing 4 amino acids. The cORF 5′ splice site hooked to the alternative is called PCAP5 and will have the 3 domains hooked to the third coding frame. The label ‘sag’ in FIG. 5 corresponds to the “Splice A” product (see below).

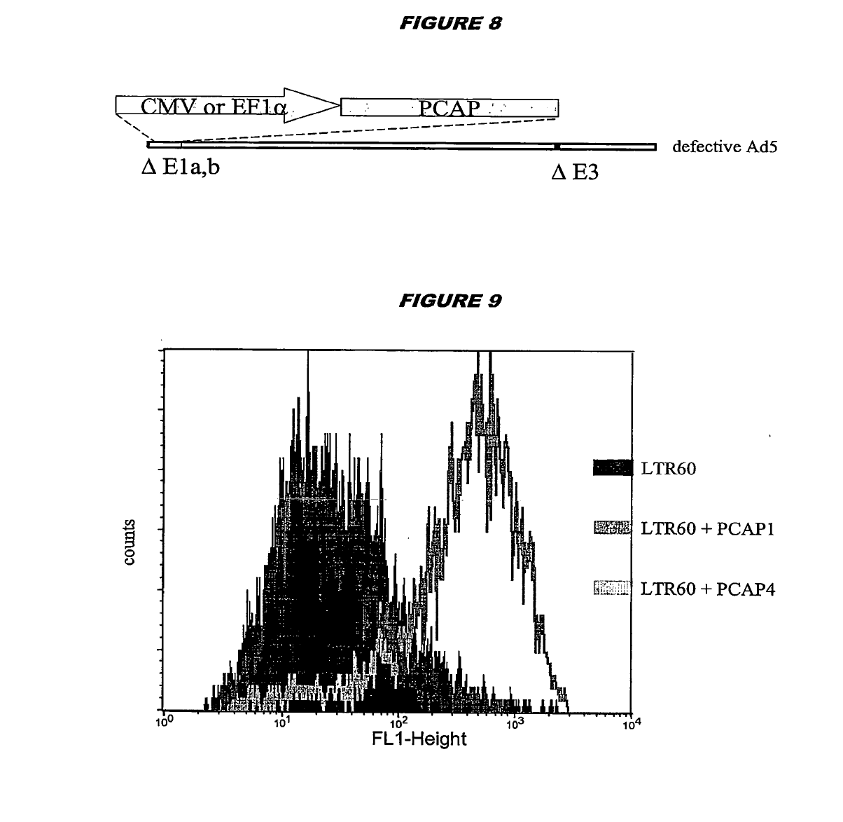

Vectors encoding cORF or the five PCAP products (FIG. 8) were co-infected with the GFP vector into primary prostate epithelial cells. Representative FACS data are shown in FIG. 9. Three PCAP products were able to activate expression, namely PCAP 4, 4a & 5, whereas PCAP 1, 2 & 3 and cORF all failed to activate expression. PCAP 4a showed the highest activity in this assay.

The interactions of PCAP4 and the non-activating PCAP products were tested by infecting cells with the GFP vector, the PCAP4 vector, and an excess of the vector encoding the non-activating product. PCAP 1, 2 & 3 and cORF could all suppress the activity of PCAP4, with cORF being the strongest dominant negative.

These data suggested that PCAV-mRNAs encode a tat homolog which contains a RNA binding domain (NLS), a polypeptide dimerization region and the third reading frame. The nucleotide sequences that make up this polypeptide product have been known since 1986, but their functional connection via alternative splicing has not previously been reported.

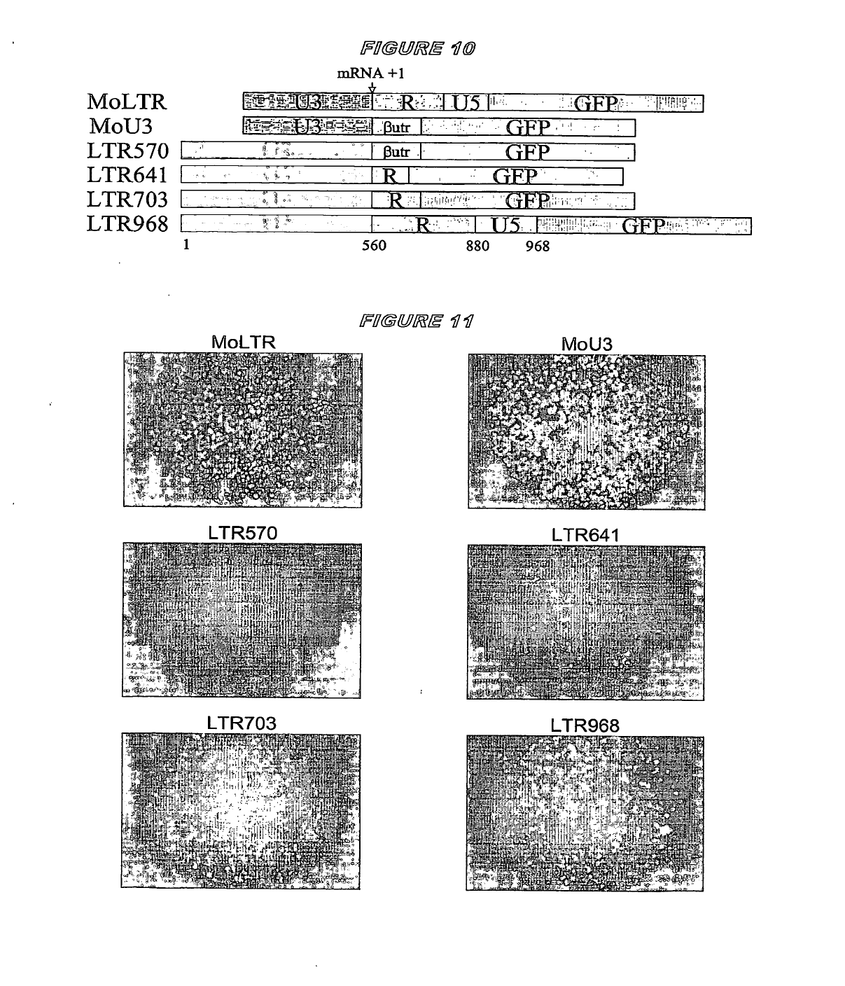

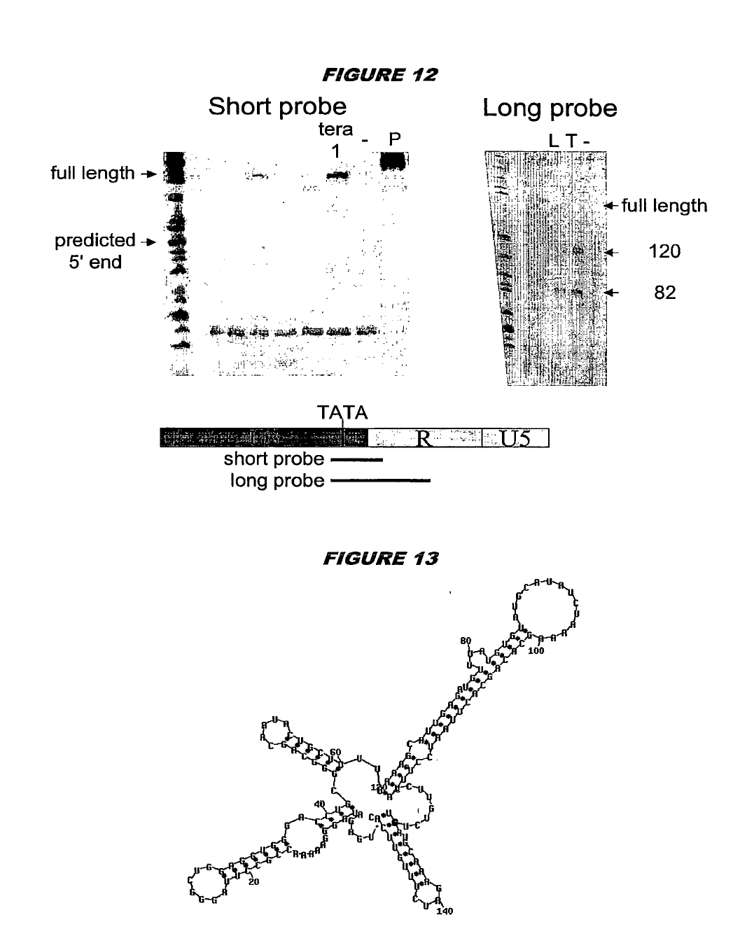

The RNA ligand of tat polypeptide in HIV is the TAR. Potential TAR sites in the LTR of PCAV-mRNAs have been investigated (FIG. 10). Deletions in the LTR showed that a region in R has a very strong effect on expression (FIG. 11), assuming that the 5′ end of PCAV-mRNA falls 30 bases downstream of the canonical TATA sequence (FIG. 12). The deletions in mutants LTR570 and LTR641 (FIG. 10) would therefore be located in the 5′ end of the PCAV mRNA and their effects would be consistent with their being the TAR. Furthermore, the first 150 nucleotides of PCAV mRNA (SEQ ID 14) are capable of forming RNAs with a highly stable secondary structure (FIG. 13), like HIV TAR.

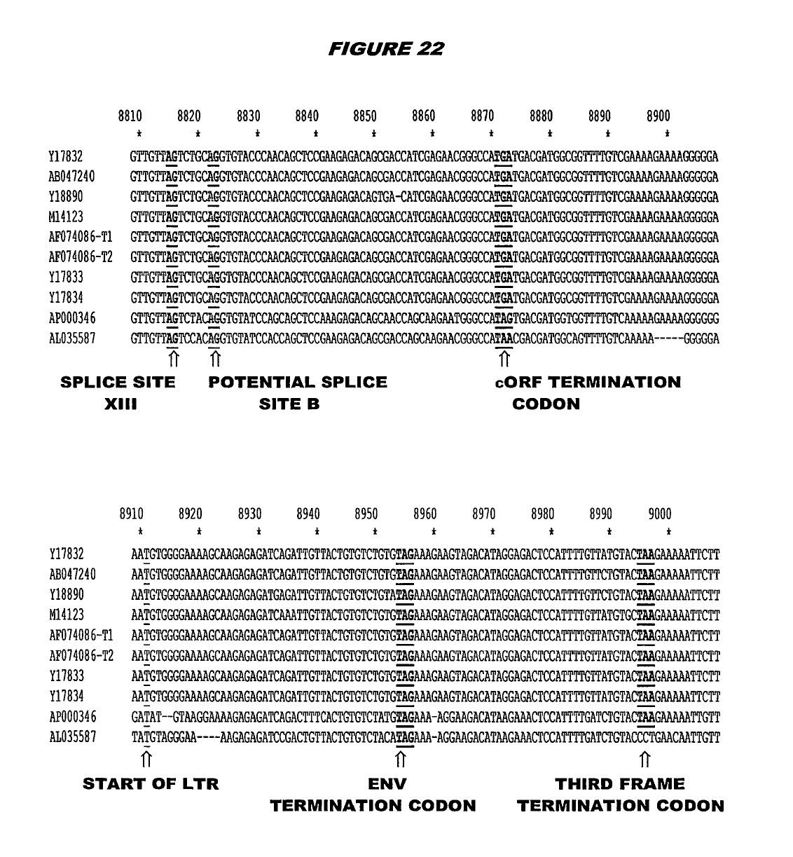

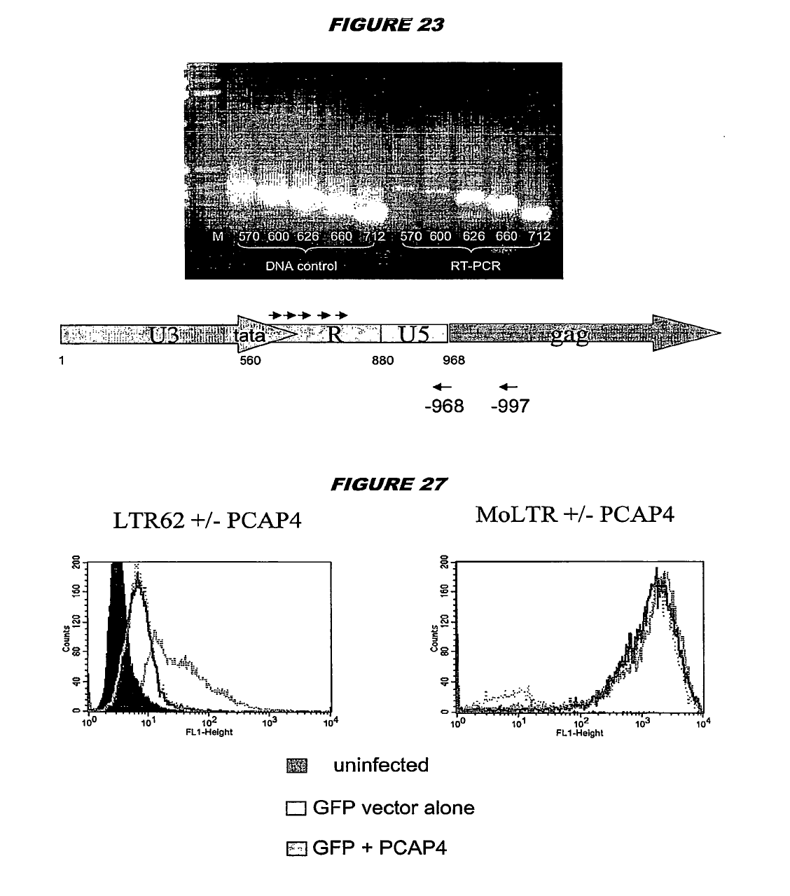

However, other work suggests that the 5′ end of PCAV-mRNA is further downstream. FIG. 23 shows the results of a RT-PCR scanning assay used to map the 5′ end. cDNA of the 5′ LTR was prepared by priming total Tera1 RNA with an antisense oligonucleotide spanning 997 to 972 in the proviral genome (SEQ ID 53). This cDNA was then divided and run in PCR analyses with an antisense primer from 968 to 950 (SEQ ID 54) combined with a sense primer from a set of primers designed to cover the likely 5′ ends: 1) 571 <SEQ ID 55>, 2) 600 <SEQ ID 56>, 3) 626 <SEQ ID 57>, 4) 660 <SEQ ID 58>, 5) 712 <SEQ ID 59>. Duplicate PCR reactions on 1 μg genomic HeLa DNA were used as a positive control, and these reactions showed all primer pairs were effective. The reactions primed with cDNA showed a marked difference between primers 600 and 626, suggesting that the 5′ end lies near position 626 in the proviral genome.

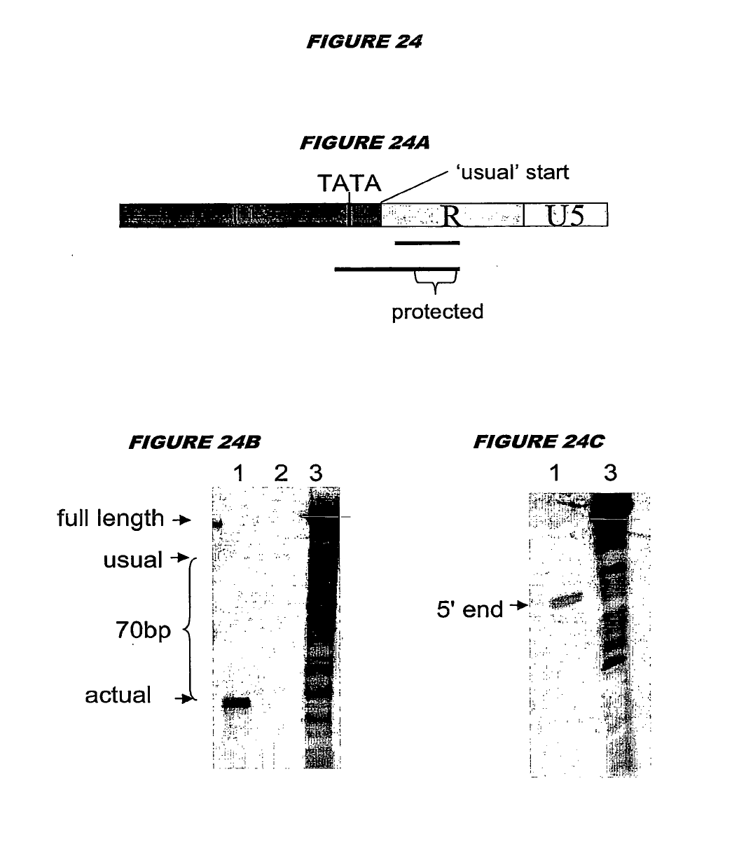

This result was confirmed using RNase protection assays (FIG. 24). Labeled antisense RNA probes covering bases (24B) 509-735 and (24C) 600-735 in the proviral genome were hybridized to total RNA from Tera1 cells and digested with RNase under standard conditions. After processing and detection by urea-containing PAGE, both probes gave 100 base products. These two results agree and show that 5′ end of HERV-K RNA is around base 635 in the proviral genome i.e. around 100 bp downstream of the TATA signal, rather than the 30 bp which is usual for TATA-dependent genes.

These two experiments suggest that the deletions used to generate the earlier data may have resulted in deletion of promoter sequences as well as transcribed sequences.

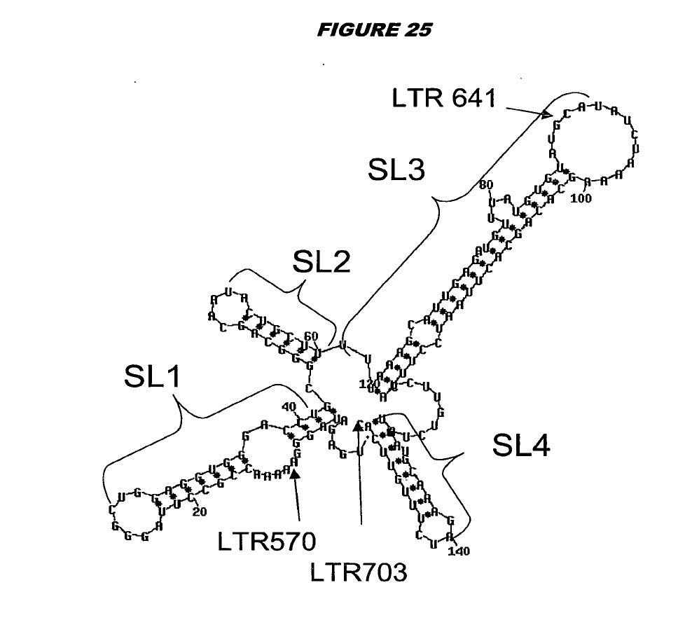

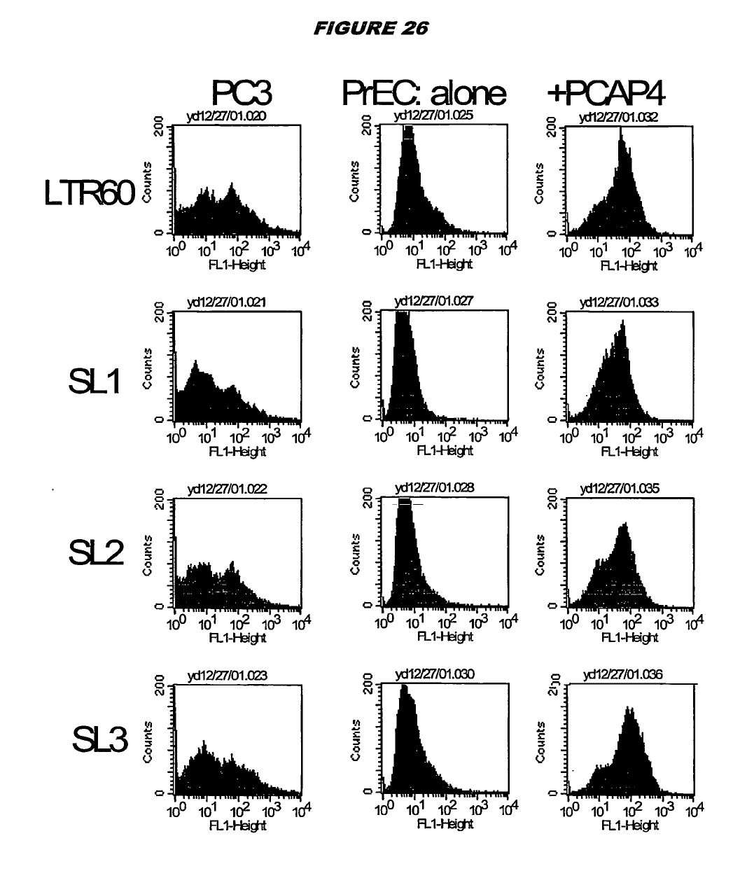

To resolve the discrepancy, stem and loop sequences of the predicted TAR structure (FIG. 13) were deleted for LTR60. If PCAV uses a tat/TAR system of transcription then these deletions would greatly diminish transcription. A deletion of each stem and loop (FIG. 25) was tested using E1-deleted adenovirus vectors with each LTR deletion mutant driving GFP. PC-3 cells were infected with each vector at a multiplicity of infection (moi) of 50 and fluorescence was measured by FACS after 3 days (FIG. 26). The full length and all deletions showed similar GFP expression. The ability of each mutant LTR to be induced by PCAP4 in a co-infection assay in PrEC cells was also tested (FIG. 26) and, again, all LTRs were induced to the same extent.

These data therefore indicate that the stem and loop regions are not involved in HERV-K LTR-driven expression, suggesting that PCAV is not controlled using a lentiviral-like tat/TAR system. Another mechanism used by complex retroviruses to activate infected cells for viral expression is the tax type, employed by HTLV I and II. Tax acts at multiple levels in infected T-cells {202}. It up-regulates HTLV transcription by binding to several transcription factors and coactivators, and deregulates the cell cycle by binding to inhibitors of CDK4/6. This combination leads to aberrant differentiation of infected cells in which the virus is activated, and is thought to be instrumental in eventually inducing adult T-cell leukemia in infected individuals. One of the hallmarks of tax-type activation is that multiple promoters respond to tax, as opposed to the high specificity of tat for the HIV TAR.

PCAP4 activates HERV-K LTR (LTR60), but not murine leukemia virus (MLV) LTR (FIG. 27). Surprisingly, PCAP4 was also found to induce expression from the HIV LTR (FIG. 28). In PrEC cells infected with an adenovirus vector carrying the HW LTR driving GFP, the GFP expression was induced by co-infection with a vector expressing PCAP4 (10 fold), and HIV LTR expression was very strongly activated by co-infection with a tat vector (100 fold), while co-infection with a lacZ vector had no effect. In further experiments on A549 cells the elongation factor 1A promoter (EF1A) was also found to be induced (FIG. 29A) whereas the CMV promoter was not (FIG. 29B).

In a separate experiment, high passage PrECs (approaching senescence) were co-infected with an adenovirus vector expressing GFP from an old-type HERV-K LTR (‘MDALTR’: SEQ ID 77), and a second vector expressing PCAP3 or PCAP4 at moi of about 20. After 3 days, the fluorescent intensity was measured by FACs and activation by PCAP3 and PCAP4 was seen (FIG. 43). In a similar experiment with LTR60 and PCAP3, however, there was no activation.

The PCAP proteins of the invention therefore seem more akin to tax than to tat, although the precise mechanism of their action is not important to the basic practice of the invention.

### PCAP2

Within the final exon in the env region of PCAV, reading frames 1 and 2 encode env and cORF, respectively (FIGS. 4 & 5). SEQ IDs 11, 28, 29 and 31 are PCAP2, which shares the same 5′ region and start codon as env, but in which the deletion found in type 1 viruses introduces a 5′ splice site which joins to a downstream 3′ splice site (FIG. 4).

The majority of the PCAP2 coding sequence is thus located after the splice, within the exon which contains the 3′ LTR. Although the +2 reading frame has no known function in HERV-K, cDNA prepared from prostate tumors included PCAP2-encoding transcripts.

Inspection of various aligned HERV-K genomes suggests that PCAP2 is a mutated form of an original protein. The protein is thus unlikely to be functioning in its original capacity, but oncogenic activity could arise through retention of a functional domain. Retention of activity by fragments is another property which matches tax rather than tat.

### PCAP2 Sub-Cellular Localization

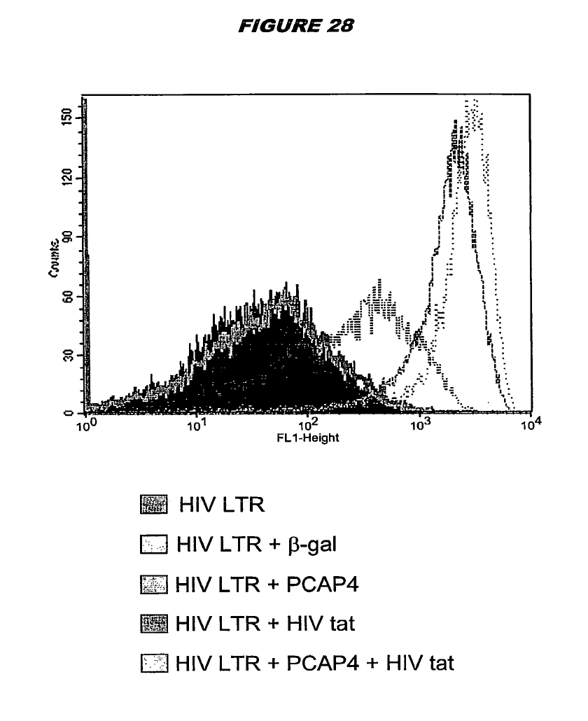

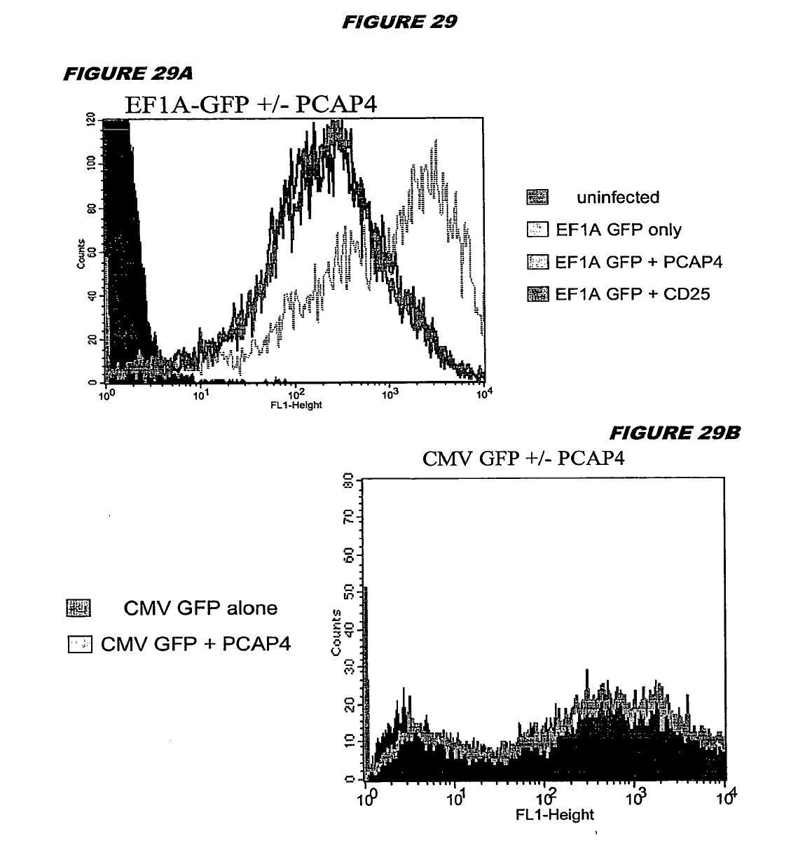

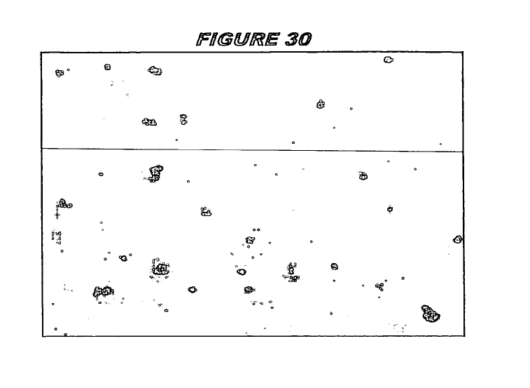

To study the subcellular localization of PCAP2, in order to better understand its role, an adenovirus expressing PCAP2 with a C-terminal V5 tag (SEQ ID 60) was used to infect primary prostate epithelial cells. The protein was not highly expressed, but was visible in the nucleoli using anti-V5 and, more diffusely, throughout the whole cell (FIG. 30). The concentration of this small protein in this cellular location shows that it is specifically interacting with something within the nucleus.

These results are consistent with the presence of NLS motifs in PCAP2.

### PCAP2 and Prostate Cell Growth

RWPE1 cells were created by immortalizing normal prostate epithelial cells with human papillomavirus 18 {203}. The cells are non-tumorgenic in nude mice and possess markers and growth characteristics of normal prostate epithelial cells.

A plasmid expressing PCAP2 from an EF1A cassette was co-transfected into RWPE1 with a puromycin selection marker. Individual resistant colonies were expanded, total RNA was prepared and positive clones were picked based on RT-PCR analysis. To assess growth characteristics, parental cells, DU145 prostate cancer cells, or selected clones were plated into matrigel plus complete keratinocyte serum-free media (complete KSFM is media with bovine pituitary extract and EGF supplements). The plated cells are shown in FIG. 31.

Normal prostate epithelial cells and RWPE1 cells migrated toward each other upon plating in matrigel, and over a week these aggregates formed hollow structures reminiscent of a gland. In contrast, DU145 cancer cells seeded solid cored colonies without apparent migration or differentiation. In the cell lines tested, both GFP lines resembled the parent RWPE1, indicating that the introduction of the vector, the selection process and the culture conditions did not change the cells. The cells expressing PCAP 1 also behaved similarly to RWPE 1. A clone expressing cORF initially aggregated like RWPE1, but then the structure dissolved and the cells took on more of a colony morphology. Three independent PCAP2 colonies failed to aggregate but instead seeded colonies like DU145 cancer cells. These data suggest that PCAP2 interferes with normal prostate cell growth and differentiation.

Using the same cell lines, the effect of PCAP2 on anchorage-independent growth of RWPE1 was tested. RWPE1 cells do not grow in 0.35% soft agar, but they do grow at lower agar concentrations (e.g. 0.3%). 1,000 cells of each type were plated in complete KSFM plus soft agar (0.35%). As shown in FIG. 32, PCAP2-expressing cells grew in soft agar to a similar extent as the positive control PC-3 cells.

### PCAP2 Expression in Tumor Tissues and Transformed Cell Lines

PCAP2 expression has been found to be associated with various tumor tissues and transformed cell lines, but not with normal non-transformed cells {204}. In particular, expression has been seen in mammary carcinoma cell lines and patient tissues.

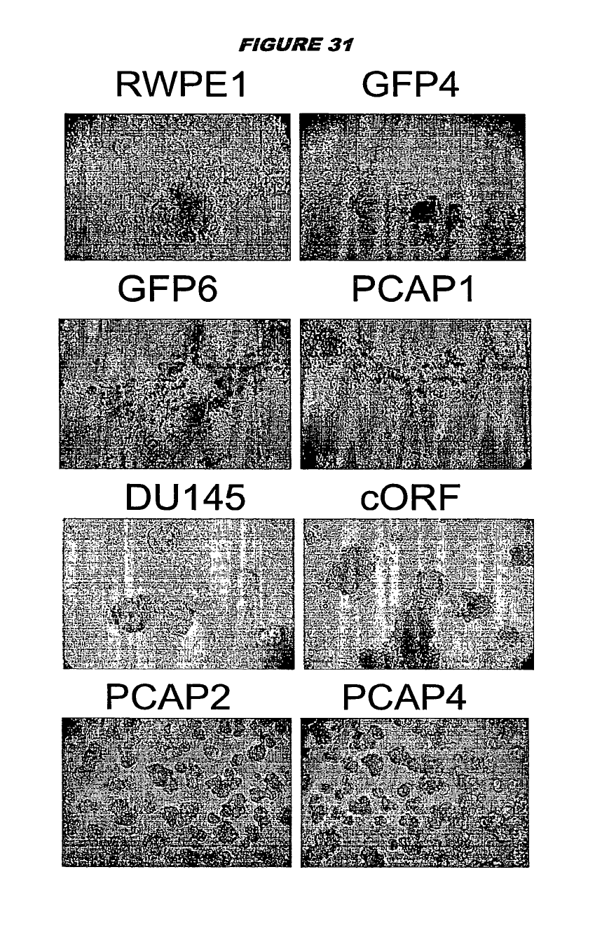

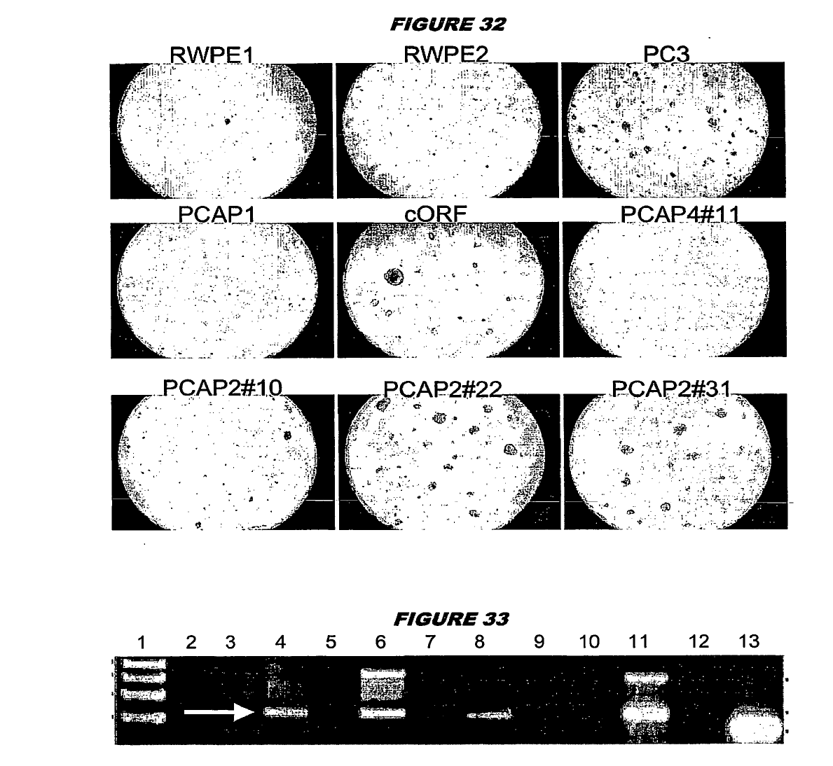

RNA extracted from tissues or cell lines as described in reference 204 has been analyzed by RT-PCR on a panel of established cell lines, tumor biopsies, lymphocytes from leukemic and normal individuals, and normal non-transformed cells. FIG. 33 shows that PCAP2 is expressed in mammary carcinoma and seminoma biopsies, as well as in transformed B cells and Tera-1 teratocarcinoma cells. Expression was seen in >90% of all transformed cell lines (n=15). PCAP2 could be detected in >45% of the samples, but it was not equally distributed among tumor types. It was most frequently seen in mammary carcinomas (52%; n=21), but was less frequently seen in germ-cell tumors (37%; n=8) and leukemia blood lymphocytes (33%; n=6). Two ovarian carcinomas tested negative. In parallel, no healthy tissue (n=14; lymphocytes, fibroblasts, gut, placenta, and stomach) expressed PCAP2. The normal diploid human fibroblasts KH5109 and non-transformed derivatives designed to express the dominant-negative mutant 175H of the p53 tumor suppressor also failed to test positive, as did immortal non-transformed human 041 fibroblasts lacking wild-type p53 and their 175H-transduced derivatives. PCAP2 expression is thus closely correlated with transformation {204}.

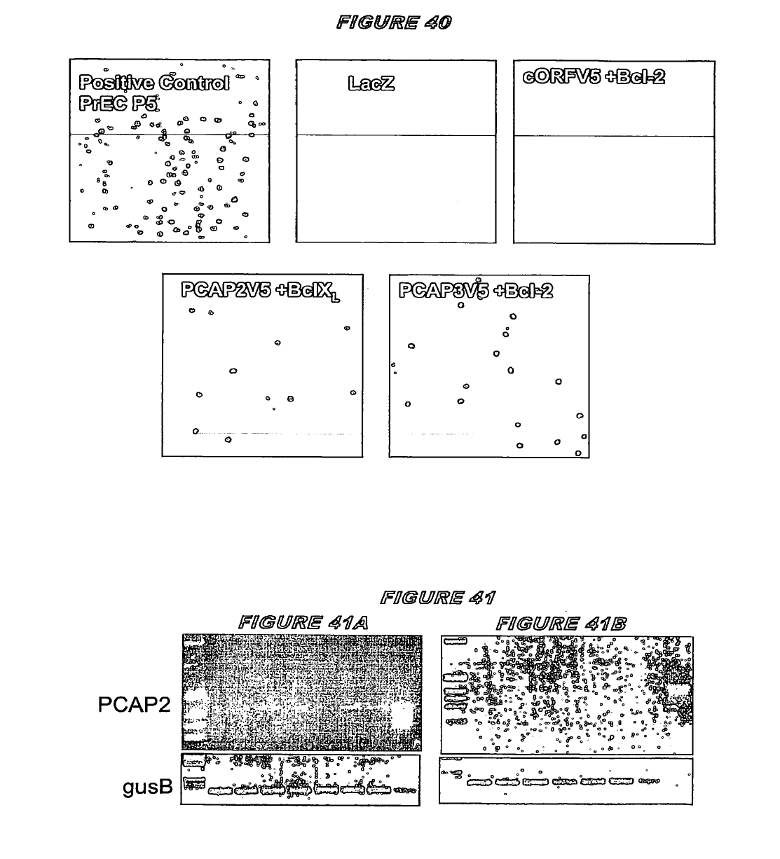

The RT-PCR results in FIG. 41 give further evidence of PCAP2 expression breast cancer. RNA was prepared and amplified from seven breast cancer biopsy samples using laser-capture microscopy of tumor tissue and peri-tumor normal tissue. cDNA was prepared with a dT primer and PCAP2 or gusB sequences were amplified using PCR for 30 (gusB) or 35 (PCAP2) cycles. PCAP2 is seen in breast cancer tissue (41A) but not in normal breast tissue (41B).

### PCAP3

SEQ IDs 12 & 36 are PCAP3, which shares the same 5′ region and start codon as env, but in which a splicing event removes env-coding sequences and shifts to a reading frame +2 relative to that of env:

| | |
|---|---|
| ATGAACTCACTGGAGATGCAAAGAAAAGTGTGGAGATGGAGACACCCCAATCGACTCGCCAGgtaaacaaa | 8253 |
| M N S L E M Q R K V W R W R H P N R L A s | |
| ...cctgttctgtctgttgttagTCTACAGGTGTATCCAGCAGCTCCAAAGAGACAGCAACCAGCAAGAATGGGCCATAG | 10480 |
| L Q V Y P A A P K R Q Q P A R M G H S | |
| TGACGATGGTGGTTTTGTCAAAAAGAAAAGGGGGGGATATGTAAGGAAAAGAGAGATCAGACTTTCACTGTGTCTATGTA | 10560 |
| D D G G F V K K K R G G Y V R K R E I R L S L C L C R | |
| GAAAAGGAAGACATAAGAAACTCCATTTTGATCTGTACTAA | 10601 |
| K G R H K K L H F D L Y * | |

PCAP3 is thus similar to PCAP2, but the shift into +2 reading frame for PCAP3 is caused by small deletions in a type 2 genome rather than the large deletion seen in type 1 genomes for PCAP2.

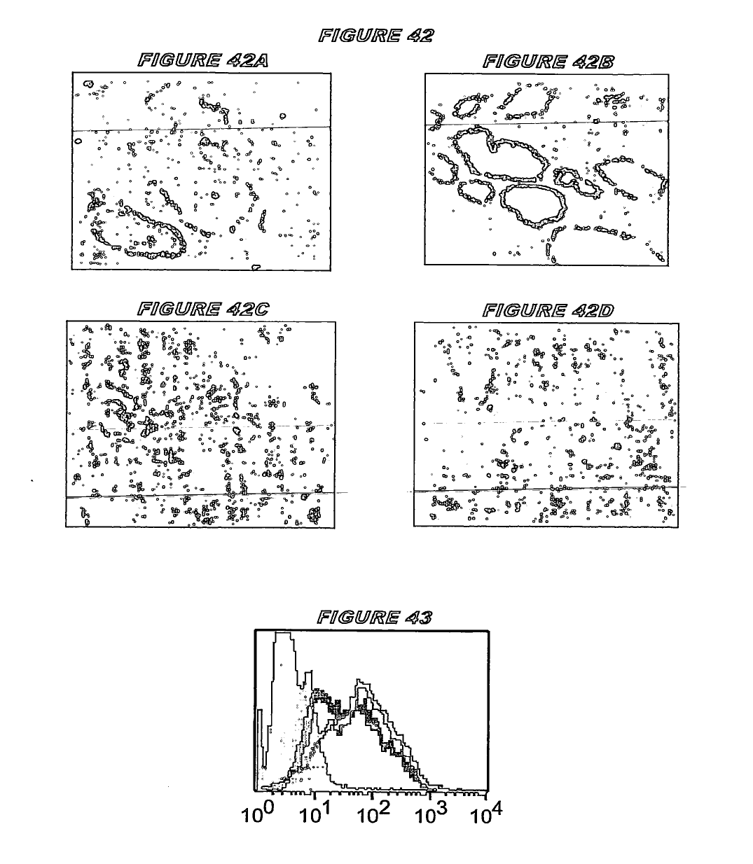

cDNA prepared from prostate cancer cell line MDA Pca-2b included PCAP3 transcripts, as did prostate cancer mRNA e.g. more than 2-fold in 79% of patient samples and more than 5-fold in 53%. These figures support the view that PCAP3 is involved in many prostate cancers. Furthermore, the figures do not reflect the whole relationship between cancer and PCAP3 expression—if patients are grouped according to Gleason grades, grade 3 tumors show high up-regulation of PCAP3 whereas more developed grade 4 tumors seem to show PCAP3 suppression (FIG. 18). A similar pattern is seen with gag expression (FIG. 42), suggestion that PCAV expression is involved in the early stages of prostate cancer.

The subcellular localization of PCAP3 was studied in the same way as described above for PCAP2. The protein was relatively stable and was seen in the nucleoplasm. The concentration of this small protein in this cellular location shows that it is specifically interacting with a target in the nucleus.

### PCAP4

As mentioned above, PCAP4 activates expression from the PCAV LTR and also from the HIV LTR. PCAP4 is generated following splicing involving a 5′ splice site 52 bases upstream of the normal cORF spice site. This splicing event causes a shift into the third reading frame in the last exon.

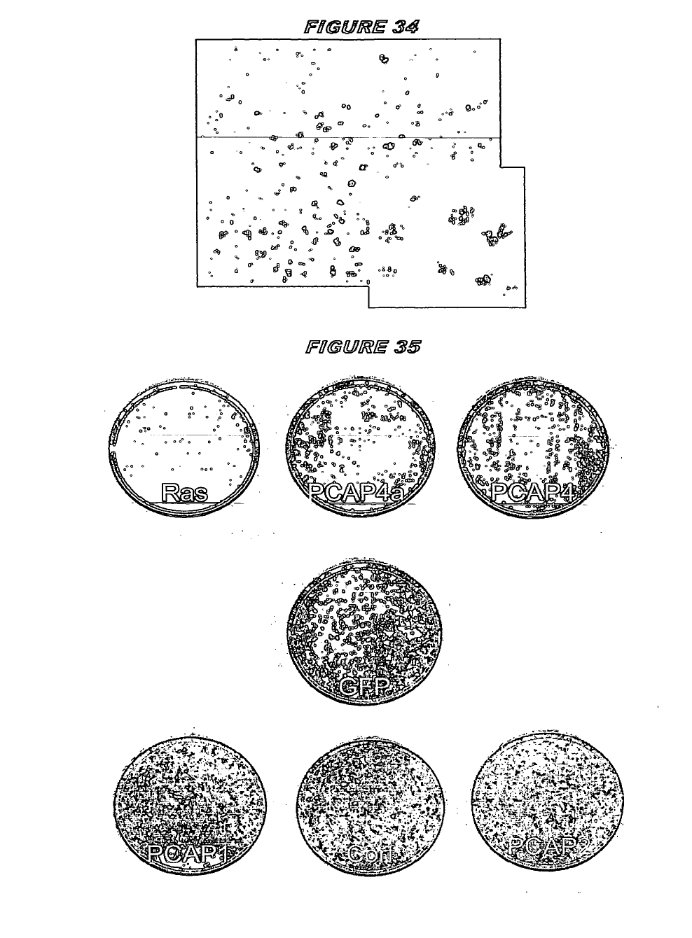

Staining of PCAP4 as described above for PCAP2 and PCAP3 shows nucleolar location (FIG. 34). In keeping with nuclear location, PCAP4 shows other activities that suggest a role in cell division. In one experiment, NIH3T3 cells were transiently transfected with expression plasmids encoding GFP, ras with a V12 activating mutation, cORF, PCAP1, PCAP2, PCAP4 or PCAP4a (a splicing variant of PCAP4). These cells were then cultured for three weeks and the overall effect on cell growth was measured by staining the cells with methylene blue (FIG. 35). Using the GFP for comparison purposes, PCAP4 and 4a induced proliferation of NIH3T3 cells in the same way as activated ras, whereas the other genes either had no effect or inhibited cell growth.

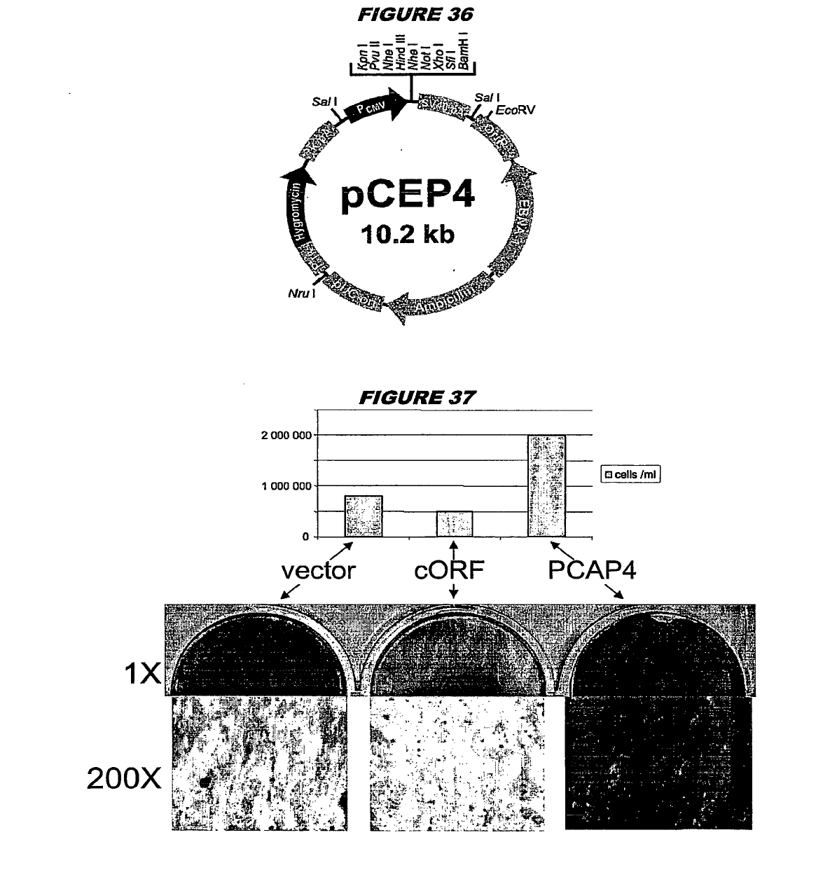

To explore this finding further, stable NIH3T3 cell lines expressing either no extra gene, PCAP4 or cORF were made by inserting the genes in pCEP4, a plasmid with a hygromycin marker (FIG. 36). Stable cell pools of each were collected, counted and allowed to grow for 4 days in duplicate wells. One well was stained and photographed, and the other was trypsin treated and counted (FIG. 37). Again, PCAP4 promoted growth of NIH3T3 cells and cORF may have slightly suppressed growth. A similar experiment with PCAP3 gave a population of cells that did not expand, but instead appeared to have off-setting high rates of death and division.

Like PCAP2, PCAP4 was able to make RWPE1 cells behave like DU145 cancer cells (FIG. 31).

### PCAP Proteins and Senescence

The above data show that PCAP2, PCAP3 and PCAP4, all of which use the third reading frame of exon 3, have a strong effect on the growth properties of immortal cell lines, including on approximately-normal human prostate epithelial cells. This oncogenic potential, combined with their expression in tumor tissue but not normal tissue, suggests a clear link with cancer.

Prostate cancer is believed to arise in the luminal epithelial layer, but normal luminal epithelial cells are capable of very few cell divisions. In contrast, NIH3T3 and RWPE1 cells are immortal. Because PCAV seems to be involved in early stages of cancer (see above), the effects of PCAP polypeptides on primary prostate epithelial cells (PrEC), which normally senesce rapidly, were tested.

Primary human epithelial cells have a very limited division potential. After a certain number of divisions the cells will enter senescence. Senescence is distinct from quiescence (immortal or pre-senescent cells enter quiescence when a positive growth signal is withdrawn, or when an inhibitory signal such as cell-cell contact is received, but can be induced to divide again by adding growth factors or by re-plating the cells at lower density) and is a permanent arrest in division, although senescent cells can live for many months without dividing if growth medium is regularly renewed.

Certain genes, particularly viral oncogenes (e.g. SV40 T-antigen) force cells to ignore senescence signals. T-antigen stimulates cells to continue division up to a further expansion barrier termed ‘replicative crisis’. Two processes occur in crisis: cells continue to divide, but cells die in parallel at a very high rate from accumulated genetic damage. When cell death exceeds division then virtually all cells die in a short period. The rare cells which grow out after crisis have become immortal and yield cell lines. Cell lines typically have obvious genetic rearrangements: they are frequently close to tetraploid, there are frequent non-reciprocal chromosomal translocations, and many chromosomes have deletions and amplifications of multiple loci {205, 206, 207}.

Gene products that lead to crisis are particularly interesting because prostate cancers exhibit high genomic instability, which could be caused by post-senescence replication. Current theory holds that prostate cancer arises from lesions termed prostatic intraepithelial neoplasia (PIN) {208}. Genetic analyses of PIN show that many of the genetic rearrangements characteristic of prostate cancer have already occurred at this stage {209}. PIN cells were thus tested for PCAV expression to determine if the virus could play a role in the earliest stages of prostate cancer. PCAV gag was found to be abundantly expressed, indicating that PCAV expression is high at the time when the genetic changes associated with prostate cancer occur. As PCAP2 and PCAP3 was seen to be expressed in prostate tumors, their roles were investigated by seeing if they are capable of inducing cell division in PrEC after senescence.

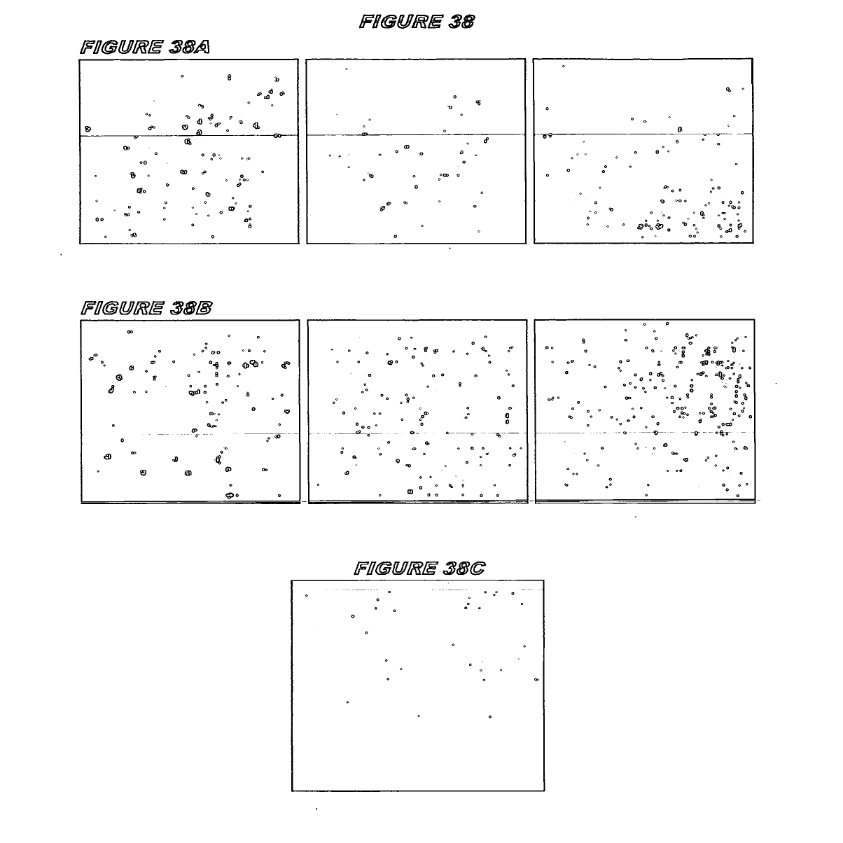

Initial attempts to select drug-resistant PrECs after transfection with PCAP expression plasmids failed. Analysis of PrEC after infection with adenovirus vectors expressing GFP, PCAP2 or PCAP3 revealed abundant cell death on day 4 post-infection in the PCAP cells. A dose-dependent increase in terminal deoxytransferase end labeling (TUNEL), to mark nuclei with nicked DNA, confirmed that the cells were undergoing apoptosis (FIG. 38). This apoptosis may explain the failure to isolate drug-resistant PrECs, and is consistent with engagement of cell division machinery by PCAP3, as an unbalanced growth signal is an inducer of apoptosis.

These results suggested that apoptosis would have to be blocked before the effect of PCAP expression in PrECs could be assessed. Plasmids encoding PCAPs 2, 3 and 4 plus neomycin markers were thus co-transfected with expression plasmids encoding either bcl-2 or bcl-X_{L} to block apoptosis. As controls, cells were transfected with plasmids expressing single proteins. After two weeks under selection, the bcl-2 and bcl-XL dishes all had numerous resistant cells that grew to fill in a fraction of the dish. When these cell were split they failed to divide further, but were viable and resembled senescent parental cells. In contrast, the cells which expressed PCAP2, PCAP3 or PCAP4 plus an anti-apoptosis protein yielded some colonies made up of small cells which divided to fill the initial plate and continued to divide when split.

In parallel to the above drug selections, the growth potential of cells was assessed. The parental PrECs went through seven population doublings before reaching senescence. In contrast, drug-resistant cells co-transfected with an anti-apoptotic gene plus a PCAP expanded well beyond the senescence point before ceasing to grow:

| PCAP product | BCL product | Doublings |
|---|---|---|
| None | None | 8 |
| 2 | Bcl-XL | 20 |
| 3 | Bcl-2 | 16 |
| 4 | Bcl-Xl | 20 |

Cells transfected with PCAP4 grew rapidly for around two weeks. Expansion of the cells then slowed and finally ceased. Concomitantly, the number of floating and dead cells increased and the appearance of the cells changed—they no longer had the regular “cobblestone” appearance of epithelial cells, but instead had several morphologies, and there were many multinucleate cells. Cells died 2 weeks later, while the cells transfected with lacZ or lacZ+bcl-2 were still alive 1 month later.

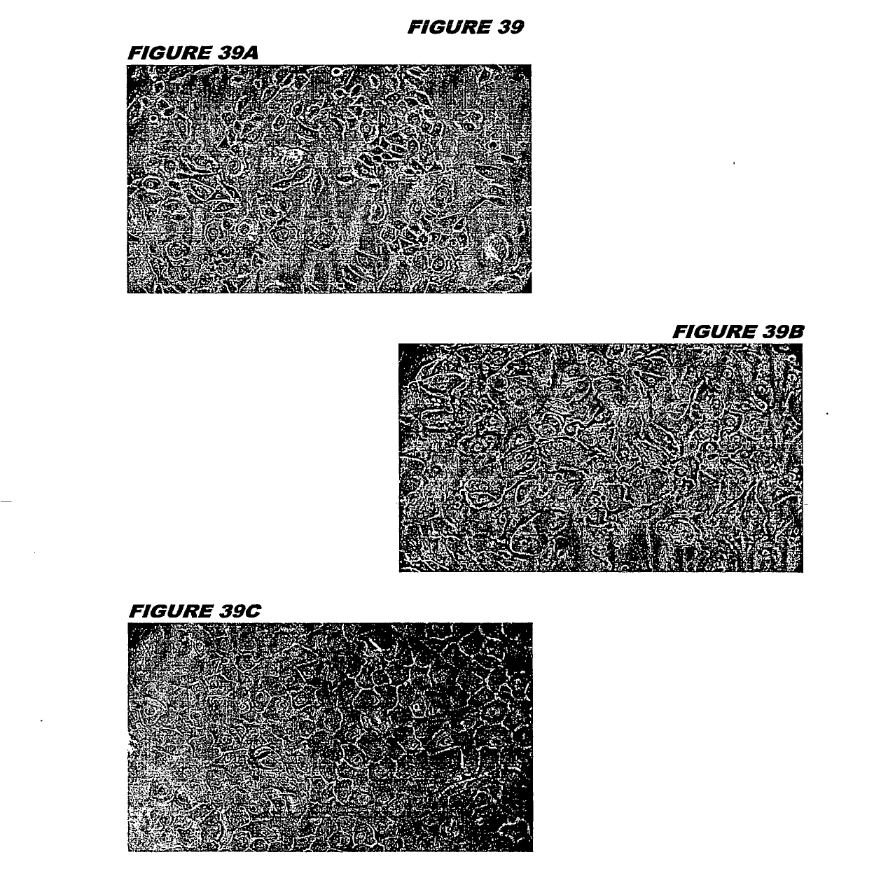

The PCAP2 and PCAP3 cells behaved similarly. FIG. 39 shows cultures maintained in supplemented prostate epithelial growth media (PrEM) renewed twice per week (including G418 for transfected cells). FIG. 39B shows the PCAP2+bcl-X_{L} cells at the stage where expansion had ceased in comparison to control cells. Senescent PrEC (FIG. 39A) and lacZ transfected cells (FIG. 39C) are regular in appearance and have a central, single nucleus in each cell, whereas the PCAP2 cells are irregularly shaped and many have multiple nuclei.

Neither senescent cells nor cells approaching crisis expand in number. One difference between them, however, is that cells approaching crisis are dividing and dying at an appreciable rate, and so cell division can distinguish between the two states. After labeling with bromo-deoxyuridine, 30% of pre-senescent PrECs were labeled, as were 10% of PrEC transfected with either PCAP2 or PCAP3 (plus anti-apoptosis proteins), but none of the senescent lacZ or cORF+bcl-2 controls were labeled (FIG. 40).

These results show that PCAP proteins are capable of inducing growth in prostate epithelial cells, and this growth could be an underlying cause of prostate cancer. The ability to drive cells past senescence is another property which matches tax rather than tat.

PCAP Products from Other HERV-K Viruses

The amino acid sequences encoded by the third reading frame of exon 3 for various HERV-Ks found in the human genome are given as SEQ IDs 78 to 277. Nucleotide sequences which encode these 200 amino acid sequences are given as SEQ IDs 278 to 477 although other nucleotide sequences, either found naturally in the human genome or designed artificially, can encode the same amino acid sequences due to codon degeneracy. The amino acid sequences are aligned below:

| | | |
|---|---|---|
| PCAP2_3rd | -----------VYPTAPKRQRPSRTGHDDD----------GGFVEKK-----RGKCGEKQ | 34 |
| 129 | -----------VYPTAPKRQRPSRTGHDDD----------GGFVEKK-----RGKCGEKQ | 34 |
| 55 | -----------VYPTARKRQRPSRTGHDDD----------GGFVEKK-----RGKCGEKQ | 34 |
| 9 | -----------VYPTAPKRQRPSRTGHDDD----------GGFVEKK-----RGKCGEKQ | 34 |
| 26 | -----------VYPTAPKRQRPSRTGHDDD----------GGFVEKK-----RGKCGEKQ | 34 |
| 54 | -----------VYPTALKRQQPSRTGHDDD----------GGFVKKK-----RGKCGEKQ | 34 |
| 98 | -----------VYPTALKRQRPSRTGHDND----------GGFVEKK-----RGKCGEKQ | 34 |
| 186 | -----------LYPTAPKRQRPSRTGHDDD----------SGFVEKK-----RGKCGEKQ | 34 |
| 224 | -------------PTAPKRQRPSRTGHDYD----------GGFVEKK-----RGKCGEKQ | 32 |
| 25 | -----------VYPTAPKRQRPSRTGHDDD----------GGFVEKK-----RGKCGEKQ | 34 |
| 219 | -----------VYPTAPKRQRPSRTGHDDD----------GGFVEKK-----RGKCGEKQ | 34 |
| 163 | -----------VYPTAPKRQRPSRTRHDDD----------GSFVEKR-----RGKCGEKQ | 34 |
| 164 | -----------VYPTAPKRQRPSRTGQDDD----------GSFVEKR-----RGKCGEKQ | 34 |
| 14 | -----------VYRTALKRQRPSRMGHDDD----------GSFVEKK-----RGKCGEKK | 34 |
| 329 | -----------VYLTALKRQRPSRMGHDYD----------GSFVEKK-----RGKCGEKK | 34 |
| 3 | -----------VFPTALKRQRPSRMGHDDD----------GGFVEKK-----RGKCGEKK | 34 |
| 123 | ------------YPTALKRQRPSRTGHDDY----------GSFVKKK-----RGKCGEKK | 33 |
| 327 | -----------VYPTALKRQRPLRTGHDDN----------GSFVEKK-----RGKCGEKK | 34 |
| 177 | -------------PTALKRQRPSRTGHDDD----------GSFVEKK-----RGKCGEKK | 32 |
| 99 | -----------VHPTAPKRQRPSRTGHDDN----------GSFVEKK-----RGKCGEKK | 34 |
| 148 | -----------VYPTAPKRQRPSRTGHDDN----------GSFVEKK-----RGKCGEKK | 34 |
| 79 | -----------VYPTAPKRQRPSRTGHDDN----------GSFVEKK-----KGKCGEKK | 34 |
| 244 | -----------VYPTAPKRQRPSRMGHDDN----------GSFVEKK-----RGKCGEKK | 34 |
| 228 | -----------VYPTAPKRQRSSRMGHDDD----------GSFVEKK-----RGKCGEKK | 34 |
| 320 | -----------VYPTAPKRQRPSRMGHDDD----------GSFVEKK-----RGKCGEKK | 34 |
| 52 | -----------VYPTAPKRQRPSRTGHDDS----------GSFVKKK-----RGKCGEKK | 34 |
| 240 | ----------EVYPTAPKRQRPSRTGHDDN----------GSFVKKK-----RGKCGEKK | 35 |
| 144 | -----------VYPTAPKRQRPSRTGHDDD----------GSFVKNK-----RGKCGEKK | 34 |
| 264 | -----------VYPTAPKRQQPSRTGHDDD----------GSFVKKK-----RGKCGEKK | 34 |
| 259 | -----------VYPTAPKRQRPSRTGHDDD----------GSFVEKK-----RGKCGEKK | 34 |
| 328 | -----------VYPTAPKRQRPSRTGHDDD----------GSFVEKK-----RGKCGEKK | 34 |
| 34 | -----------VYPTAPKRQRPSRTGHDDD--------- GGFVEKQ-----RGKCGEKK | 34 |
| 321 | -----------VYPTAPKRQRPSRTGHDDD----------GSFVEKQ-----RGKCGEKK | 34 |
| 27 | -----------VYPTAPKRQRPSRTGHDDS----------GGFVEKK-----RGKCGEKK | 34 |
| 47 | -----------VYPTAPKRQRPSRTGHDDN----------GGFVEKK-----RGKCGEKK | 34 |
| 36 | -----------VYPTAPKRQRPSRTGHDDD----------GGFVEKK-----RGKCGGKN | 34 |
| 108 | -----------VYPTAPKRQRPSRTGHDDD----------GGFVEKK-----RGKCGGKK | 34 |
| 15 | -----------VYPTAPKRQRPSRTGHDDD----------GGFVEEK-----RGKCGAKK | 34 |
| 102 | ----------EVYPIAPKRQRPSRTGHDDD----------GGFVEKK-----RGKCGEKK | 35 |
| 322 | -----------VYPAAPKRQRPSRTGHDDD----------GGFVEKK-----RGKCGEKK | 34 |
| 31 | -----------VYPTAPKRQRPSRTGHDDD----------GGFVEKK-----RGKCGEKK | 34 |
| PCAP3 | -----------VYPAAPKRQQPARMGHSDD----------GGFVKKK-----RGGYVRKR | 34 |
| 172 | -----------VYPAAPKRQQPARMGHSDD----------GGFVKKK-----RGGYVRKR | 34 |
| 302 | ---------LQVYPAAPERQRPARRDHDDH----------GGFVKKK-----SGKCREKR | 36 |
| 303 | ---------LQVYPAAPERQRPARRDHDDH----------GGFVKKK-----SGKCREKR | 36 |
| 296 | ---------LQVYPAAPERQRPGRRGHDDH----------GGFVKKK-----SGKCREKR | 36 |
| CHR8_3rd | ---------LQVYPAAPERQRPARRGHDDH----------GGFVKKK-----SGKCREKR | 36 |
| 208 | -----------VYPAAPERQRPVRRGHDDD----------GGFVKKK-----RGKCREKR | 34 |
| 212 | ---------- LYPAAPERQRPARRGHDDG----------GGFFKTK-----RGICREKK | 34 |
| 20 | -----------VYPTAPKRQRPSRTGQYDD----------GSFVKKKRGRKEKGEMWGKE | 39 |
| 32 | -----------VYPTAPKRQRPSRTGHDDD----------GGFVEKK----EKGEMWGKE | 35 |
| 140 | -----------------RRERPSRTSHDDN----------GGFVEKK------GEMWGKE | 27 |
| 156 | -----------VYPIAPKRQRTSRTGHDDN----------GGFVEKK------REMWGKE | 33 |
| 282 | -----------VYPTAPKRQRPSRTGHDDD----------RGFVKKK-----WGKMWGKK | 34 |
| 7 | -----------VYPAAPKRQRPSRTSHDDD----------GGLSKRK---------WGNV | 30 |
| 17 | -----------VYPTAPKRQRPSRTGHDDD----------GGFVEKK------RRKSGEK | 33 |
| 201 | -----------VYPTAPKRQRPSRTGHDDD----------GGFVEKR------RGKCGEK | 33 |
| 254 | -----------LYPTAPKRQRPLRMGHDAD----------GGFVEKK-----RGKCGEKK | 34 |
| 312 | -----------LYPTAPKRQRPSRMGHDDD----------GGFVKKK-----RGKCGGKR | 34 |
| 239 | -----------VYPAAPKRQRPSRTGHDDD----------GSFVKNK-----RE-NVGKR | 33 |
| 319 | -----------VYPTAPKRQRPSRMGHDDD----------GGFVEKK-----GG-NVEKR | 33 |
| 242 | -----------VYPTAPKRQRPSRTGHDERAM-----MTMAVLLKRK-----GG-NAGKR | 38 |
| 333 | -----------VYSTAPKRQRPGRMGHDD--------V--AVLSKRK-----GG-NVGKR | 33 |
| 213 | -----------VYPTAPKRKRPSRMGHDDN----------GGFVEKK-----RG-NVGKR | 33 |
| 190 | -----------VYPTSPKRQRPSRTGHDDD----------GGFVEKK-----RG-NVGKR | 33 |
| 84 | -----------VYPTAPKKQQPSIMGHDDD----------GGFVKKK-----RGKCGEKR | 34 |
| 149 | -----------VYPTAPKRQQPSRTGHDDD----------GSFVKKK-----RGKCGEKR | 34 |
| 135 | -----------VYPTAPKRQRPSRTGHDDD----------GGFVQKK-----RGK-WEKR | 33 |
| 226 | -----------VYPTAPKRQRPSRTGHDDD----------GSFVIKK-----RGKRGEKR | 34 |
| 51 | -----------VYPTAPKRQRPSRTGHDDD----------GGFVEKK-----RGKCGEKR | 34 |
| 71 | -----------VYPTALKRQRPSRTGHDDD----------GGFVEKK-----REKCGEKK | 34 |
| 176 | -----------VYPTALKRQRPSRTGHDDD----------GSFVEKK-----RGKCGEKR | 34 |
| 261 | -----------VYPTAWKRQRPSRMGHDDD----------GGFVEKK-----RGKCGENR | 34 |
| 94 | -----------VYPTAPKRQRLSRTGHDDD----------GGFVEKK-----RGKCGEKK | 34 |
| 233 | -----------VYPTAPKRQRPSRTGHDDN----------GGFVEKK-----RGKCGEKK | 34 |
| 69 | ---------LQVYPTALKRQQPSRTGHDDD----------GSFVEKK-----RGKCGEKK | 36 |
| 183 | -----------VYPTAPKRQRPLRTGHDDD----------GSFVEKK-----RRKCGEKR | 34 |
| 268 | -----------VYPTAPKRQRPSRTGHDDD----------GAFVEKK-----RGKCGEKK | 34 |
| 19 | -----------VYPTAPKRQRPSRTGHDDD----------GGFVRKK-----RGKCGEKK | 34 |
| 246 | -----------VYATALKRQRPSRTGHDDD----------GGFVEKK-----RGKCGEKK | 34 |
| 335 | -----------VYPTAPKRQRPSRTGHDDD----------GGFAEKK-----RGKCGEKK | 34 |
| 116 | -----------VYPTAPKRQRPSRTGHDDD----------GGFVEKK-----RGKCGEKK | 34 |
| 63 | -----------VYPTAPKRQRPSRKGHDDD----------GGFVEKK-----RGKCGEKK | 34 |
| 73 | -----------VYPTAPKRQPPSRTGHDDD----------GGFVLKK-----RGKCGEKK | 34 |
| 74 | -----------VYPTAPKRQRPSRTGHDDD----------GGFVEKK-----RGKCGEKK | 34 |
| 109 | -----------VYPTALKRQRPSRTGHDDD----------GGFVEKK-----RRKCGEKK | 34 |
| 111 | -----------VYPTAPKRQRPSRTGHDDD----------GGFVEKK-----RRKCGEKK | 34 |
| 83 | -----------VYPTAPKRQRPSRTGSDDD----------GGFVEKK-----RGKCGEKK | 34 |
| 235 | -----------------RRDRPWRTGHDDD----------GGFVEKT-----RGKCGEKK | 28 |
| 332 | -----------MYPTPLKRQRPWRTGHDDN----------GGFVEKK-----RGKCGEKK | 34 |
| 251 | -----------VYPTALKRQRPWRTGHDDD----------GGFVEKK-----RGKCGEKK | 34 |
| 162 | -----------VYPTAPKRQRPWRTGHDDD----------GGFVEKK-----RGKCGEKK | 34 |
| 315 | -----------VYPTALKRQRPSRTGHDDD----------GGFVEKK-----RGKCGEKK | 34 |
| 1 | -----------VYPTAPKRQQPSRTGHDND----------GSFVEKR-----RGKCGEKK | 34 |
| 203 | -----------VYPTAPKRQQPSRTGHDDD----------GSFVEKK-----RGKCGEKK | 34 |
| 13 | -----------VYPTAPKRQQPSRTGHDDD----------GCFLEKK-----RGKCGEKK | 34 |
| 96 | -----------VYPTAPKRQQPSRTGHDDD----------GGFVKNK-----RGKRGEKK | 34 |
| 217 | -----------VYPTAPKRQQPSRTGHDDD----------GGFVEKK-----RGKRGEKK | 34 |
| 198 | ------------YPTAPKRQQPWRTGLDDL----------GGFFEKK-----RGNFGEKK | 33 |
| 199 | -----------VYPTAPKRQQPWRTGHDDH----------GGFVEKK-----RGKCGEKK | 34 |
| 269 | -----------VYPTAPKRQQPLRTGHNDD----------GGFVEKK-----RGKYGEKK | 34 |
| 35 | -----------VYPTAPKRQQPSRTGHDED----------GGFVERK-----RGNCGEKK | 34 |
| 24 | -----------VYPTAPKRQQPSRMGHDDD----------GGFVKKK-----RGKCGEKK | 34 |
| 113 | ----------EVYPTSPKRQQPSRMGHDDD----------GGFVAKK-----RGKCGEKK | 35 |
| 130 | -----------VYPTAPKRQQPSRMGHDDN----------GGFVEKK-----RGKCGEKK | 34 |
| 318 | -----------VYPTAPKRQQPSRMGHHDD----------GGFVEKK-----RGKCGEKK | 34 |
| 29 | -----------VYPTARKRQQPSRTGHDDD----------GGFVVKK-----RGKCGEKK | 34 |
| 232 | -----------VYPTALKRQQPSRTGHDDD----------GGFVEKK-----RGKCGEKK | 34 |
| 80 | -----------VYPTAPKRQQPSRTGHDDD----------GGFVKKK-----RGKCGEKK | 34 |
| 160 | -----------VYPTAPKRQQPSRTGHDDD----------GGFVQKK-----RGKCGEKK | 34 |
| 68 | -----------VYPTAPKRQRPSRTGHDDD----------GGFVEKK-----REKCGEKK | 34 |
| 249 | -----------VYPTAPKRQQSSRTGHDDD----------GGFVEKK-----REKCGEKK | 34 |
| 231 | -----------VYPTAPKRQRPSRMAHDDD----------GGFVENK-----SGKCGEKK | 34 |
| 234 | -----------VYPTAPKRQRPSRTGHDDD----------GGFVEKK-----RGKCGEKK | 34 |
| 42 | -----------VYPAAPKRQRPSRTSHDDD----------GSFVKKK-----RVMWG--K | 32 |
| 236 | -----------VYLAAPKRQRPSRTSHDDN----------GGFVKKK-----RGKCGEEK | 34 |
| 16 | -----------VYPTAPKRQQPSRNSHDDD----------GGFV-EK-----GEMWG--K | 31 |
| 134 | ------------YPTAPKRQRPSRKSHDDD----------GGFVEKK-----RGKYGEKK | 33 |
| 90 | -----------VYPTAPKRQRPSRTGHDDD----------GGFVEKK-----MGKFGEKK | 34 |
| 28 | -----------VYPTAPKRQRPSRMGHDDD----------GGFVEKK-----RGKCGEKK | 34 |
| 200 | -----------VYPTAPKRQRPSRMGHDDD----------GGFVEKK-----RGKCGEKK | 34 |
| 23 | -----------VYPTAPKRQRPSRMGHDDY----------GGFVEKK-----RGKCGEKK | 34 |
| 37 | -----------VYPTAPKRQRPSRMGHDDD----------GGFVEKK-----RGKCGEKQ | 34 |
| 324 | -----------VYPTAPKRQRPSRMGHDDD----------GGFVEKK-----RGKCGEKK | 34 |
| 53 | -----------VYPTAPKRQRPSRMGHDDD----------GGFVEKQ-----RGKCREKK | 34 |
| 191 | -----------VYPTAPKRQRPLRMGHGDD----------GGFVEKK-----RGKCREKK | 34 |
| 117 | -----------VYPTAPKRQRPLRMGHDDD----------GGFVEKK-----MGKCGEKK | 34 |
| 230 | -----------VYPTAPKRQRPLRMGHDDD----------GGFVEKK-----RGKCGEKK | 34 |
| 120 | -----------VYPTAPKRQRPWRMGHDDD----------GGFVEKK-----RGKCGEKK | 34 |
| 121 | -----------VYPTAPKRQRPWRMGHDDD----------GGFVEKK-----RGKCGEKK | 34 |
| 252 | -----------VYPTAPKRQRPSRAGHDDD----------RGFVEKK-----RGKCGEKK | 34 |
| 258 | -----------VYPTAPKRQRPSRAGHDDD----------GGFVEKK-----RGKCGEKE | 34 |
| 314 | -----------VYPTAPKRQRPSRRGHDDD----------GGFVEKK-----RGKCEEKK | 34 |
| 323 | -----------VYPTAPKRQRPSRRGHDDD----------GGFVKKK-----RGKCGEKK | 34 |
| 131 | -----------VYPTAPKRQRPSRTGHDDD----------GGFVEKK-----RGKCREKK | 34 |
| 326 | -----------VYPTAPKRQRPSRTGHDDD----------GGFVEKK-----RGKCREKK | 34 |
| 151 | -----------VYPTAPKRQRPLRTGHDDD----------GGFVEKK-----RGKCGEKK | 34 |
| 248 | -----------VYPTAPKRQRPSRTGHDDD----------GGFVEKK-----RGKCGENK | 34 |
| 75 | -----------VYSTAPKRQRPSRTGHDDD----------GGFVEKK-----RGKCGEKK | 34 |
| 56 | ---------------KP--RRTKTQH--------TRISGTHS------------TCGEKQ | 23 |
| 311 | -------------PLCP--RLKQSSR--------LSLSSSRD------------CCGEKQ | 25 |
| 166 | -----------PPEQRP--REMNGCH--------SGPDPRHSQE---------GPCGEKK | 30 |
| 174 | -----------PPEQRP--REMNGCH--------SGPDPRHSQE---------GPCGEKK | 30 |
| 155 | -----------PPEQRP--REMNGCH--------SGPDLRHSQE---------GPCGEKK | 30 |
| 82 | -----------PSEQRP--RETNGCH--------SGPDPRHSQE---------GPCGEKK | 30 |
| 245 | ----------------------------P----GNPRRKLPQGQG--------HHCGEKQ | 20 |
| 310 | LHPLSPSQLAPPQPGHPAWATPSDCHNPR----AYGQDELHQVKM--------VECGEKQ | 48 |
| 87 | ---------------SPSAQRPPRLGGVPNSSLRTGHDDDGGFVEWR-----GGKCGEKI | 40 |
| 189 | ---------------SPSAQRPPRLGGVPNSSLRTGHDADGGFVEWK-----RGKCGEKI | 40 |
| 132 | ----------------PSGRCAQQLI-------EKGHDDNGGLVEWR-----RGKCGEKR | 32 |
| 317 | ------------WPAAPSGRCTQQL--------RTGHDDNGGFVEWK-----GGKGGEKI | 35 |
| 125 | ----------------APTRQPPCLRGVPNSSLRTGHDDDGGFVEQK-----RGKCREKK | 39 |
| 146 | -----------VYPAAP--KRQRPLR--------TGHDDDGGFVEKK-----RGKCGEKK | 34 |
| 256 | -----------VYPTAP--KRQRPLR--------TGHDDDSGFVEKK-----RGKCGEKK | 34 |
| 210 | -----------VYPTAPKRQRPSRTGHDDD----------GGFVEKK-----RGKCGEKK | 34 |
| 211 | -----------VYPTAPKRQRPSRTGHDDD----------GGFVEKK-----RGKCGEKK | 34 |
| 265 | -----------VYPTAPKRQRPWRTGHDDD----------GGFVKKK-----RGKCGEKK | 34 |
| 93 | -----------VYPTAPKRQRPLRMGHDDD----------GSFVKKK-----RGKCGEKK | 34 |
| 106 | -----------VYPTAPKRQRPLRMGHDDD----------GGFVKKK-----RGKCGEKK | 34 |
| 291 | -----------VYPTAPKRQRPLRRGHDDD----------GGSVKKK-----RGKCGEKK | 34 |
| 12 | -----------VYPTAPKRQRPLRTGHDDD----------GGFVKKK-----RGKCGEKK | 34 |
| 223 | -----------VYPTAPKRQRPTRTGHDDD----------GGFVKKK-----RGKCGEKK | 34 |
| 86 | -----------VYPTAPKRQRPSRTGHDDD----------GGFVKKK-----RGKCGEKK | 34 |
| 88 | -----------VYPTAPKRQRPSRTGHDDD----------GGFVKKK-----RGKCGEKK | 34 |
| 205 | -----------VYPTAPKRQRSSRTGRDND----------GGFVKKK-----RGKCGEKK | 34 |
| 126 | -----------VYPTAPKRQRPSRTGHDDD----------GGFVKKK-----RGKCGEKK | 34 |
| 165 | -----------VYPTAPKRQRPSRTGHEDD----------GGFVKKK-----RGKCGEKK | 34 |
| 330 | -----------VYPTVPKRQRPSRKGHEDD----------GCFVKKK-----RGKFGEKK | 34 |
| 247 | -----------VYPTSPKRQRPSRTGHDDD----------GGFVKKK-----RGKCGEKK | 34 |
| 4 | -----------VYPTAPKRQQPSRTGHDDD----------GGFVKKK-----RGKCGEKK | 34 |
| 10 | -----------VYLTAPKRQRPSRTGHDDD----------GGFVKKK-----RGKCGEKK | 34 |
| 81 | -----------VYPTASKRQPPSGTDHDDD----------GGFVKKK-----RGKCGEKK | 34 |
| 115 | -----------VYPTASKRQPPSGTDHDDD----------GSFVKKK-----RGKCGEKE | 34 |
| 175 | -----------VYPTAVKRQRPSRTGHDDD----------GGFVKKK-----RGKCGEKK | 34 |
| 275 | -----------VYPTARKRQRPSRTGHDDD----------GGFVKKK-----RGKCGEKK | 34 |
| 270 | -----------VYPIALKRQRPSRTGHDDD----------GGFVKKK-----RGKCGEKK | 34 |
| chrY_3rd | ---------LQVYPAAPERQQPARTGHDDYGS----------FVKKK-----RDICREKK | 36 |
| 272 | ---------LQVYPAAPERQRLARTDHDDDGG----------FVKKK-----RGICREKR | 36 |
| 308 | ---------LQVYPAAPERQQPAKTGHNDYGG----------FVKKK-----RGICTAKK | 36 |
| 40 | ---------LQVYPTAPKRQQPARTGHNDDGS----------FVKKK-----RGICREKK | 36 |
| 170 | -----------VFTTAEQGRTPAPGTQRDFAKG-----MDLAGPRGC-----L--CREKK | 37 |
| 266 | -------------PAWPTWRNPVSTKNTKLAR---------HG-AAC-----LQSCREKK | 32 |
| 5 | ---------LQVYPAAPKRERPVRTGHDDDGG----------FLKKK-----RGICREKK | 36 |
| 64 | ---------LQVYTTAPERQRPARTGHDDDGG----------FVKKK-----RGKCREKK | 36 |
| 221 | -----------VYPAASETQRPARTGHDDDGG----------FVKKK-----RGICREKK | 34 |
| 43 | ---------LQVYPAAPERQRPGRRGHDDGGG----------FVKTK-----RGICRGKK | 36 |
| 50 | ---------LQVYPAAPERQRPGRRGHDDGGG----------FVKTK-----RGICRGKK | 36 |
| 60 | ---------LQVYPAAPERQRPARRGHDDGGG----------FVKTK-----MGICREKK | 36 |
| 192 | ---------LQVYPAAQERHRPARRGHDDGGG----------FVKTK-----RGIYREKK | 36 |
| 57 | -----------VYPAAPERQRPARRGHDDGGG----------FVKTK-----RGICRVKK | 34 |
| 187 | -----------------DSDRPERRGHDDGGG----------FVKTK-----RGICREKK | 28 |
| 293 | -----------VYPAAPERQRPARRGHDDGGG----------FVKMK-----RGICREKK | 34 |
| 299 | ---------LQVYPAAPERQRPARRGHDDGGG----------FVKTK-----RGICREKK | 36 |
| 292 | -----------VYPAAPERQRPARRGHDDGGG----------FVKTK-----RGICREKK | 34 |
| 207 | -----------VYPAAPERQRPARRGHDDGGG----------FVKKK-----RGICREKK | 34 |
| 178 | -----------VYPAAPERQRPARRGHNDGGG----------FVKKK-----RGICREKK | 34 |
| 152 | -----------VYAAALERQRPARNGHDDDGG----------FVKKK-----RGIYREKK | 34 |
| 195 | -----------VYPAATEKQRPARTGHDDDGG----------VVKKK-----RGKCREKK | 34 |
| 138 | ---------LQVYPAAPKRQRPLRMGDDDDGG----------FVKKK-----RGKCGEKK | 36 |
| 204 | -----------VYPAAPERQRPARMGHDDDGG----------FVKKK-----RGKCREKK | 34 |
| 22 | -----------VYPAAPKRQRPVRMGHNDDVS----------FVKKK-----RGICREKK | 34 |
| 11 | -----------VYPTALKRQRPKRMGHDDYGS----------SVKKK-----RGICRGKK | 34 |
| PCAP2_3rd | ERSDCYCVCVERSRHRRLHFVLY------------------------------------- | 57 |
| 129 | ERSDCYCVCVERSRHGRLHFVLY------------------------------------- | 57 |
| 55 | ERSDCYCVCVERSRHRRLHFVLY------------------------------------- | 57 |
| 9 | ERSDCYCVCVERSRHGRLHFVM-------------------------------------- | 56 |
| 26 | ERSDCHCVCVERSRHGRLHFVMY------------------------------------- | 57 |
| 54 | ERSDCHCVCVERSRHRRLHFVMY------------------------------------- | 57 |
| 98 | ERSDCHCVCVERSRHRRLHFVMY------------------------------------- | 57 |
| 186 | ERSDCYCVCVERSRHRRLHFVMY------------------------------------- | 57 |
| 224 | ERSDCCCVCVERSRHRRLHFVMY------------------------------------- | 55 |
| 25 | ERSDCYCVCVERSRHRRLHFVMY------------------------------------- | 57 |
| 219 | ERSNCYCVCVERSRHRRLHFVM-------------------------------------- | 56 |
| 163 | ERSDCYCVCVERSRHRRLHLVMY------------------------------------- | 57 |
| 164 | ERSDCYCVCVERSRHRRLHFLMY------------------------------------- | 57 |
| 14 | ERSDCYCVCVERSRHRRLHFVLY------------------------------------- | 57 |
| 329 | ERSDCYCVCVERSRHRRLHFVLY------------------------------------- | 57 |
| 3 | ERSDCYCVCVERSRHRRLHFVLY------------------------------------- | 57 |
| 123 | ERSDCYCVCVERSRHSRLHFVLY------------------------------------- | 56 |
| 327 | ERSDCYCVCVERSRHRRLHFVLY------------------------------------- | 57 |
| 177 | ERTDCYCVCVERSRHRRLHFVLY------------------------------------- | 55 |
| 99 | ERSDCYCVCVERSRHRRLHFVLY------------------------------------- | 57 |
| 148 | ERSDCYCVCVERSRHRRLHFVLY------------------------------------- | 57 |
| 79 | ERSDCYCVCVERSRHRRLHFVLY------------------------------------- | 57 |
| 244 | ERSDCYYVCVERSRHRRLHFVLY------------------------------------- | 57 |
| 228 | ERSDCYCVYVERSRHRRLHFVLY------------------------------------- | 57 |
| 320 | ERSDCYCVCVERSRHRRLHFVLY------------------------------------- | 57 |
| 52 | ERSDCYCVCVERSRHRRLRFVLY------------------------------------- | 57 |
| 240 | ERSDCYCVCVERRRHRRLHFVLYQEMFFCLGMLLIYNLTPNPLLSETCAV---------- | 85 |
| 144 | ERSDCCCVCVERSRHRRLHFVLY------------------------------------- | 57 |
| 264 | ERSDCCCVCVERSRHRRLHFVLY------------------------------------- | 57 |
| 259 | ERSDCSCVCVERSRHRRLHFVLY------------------------------------- | 57 |
| 328 | ETSDCYCVCVERSRHRRLHFVLY------------------------------------- | 57 |
| 34 | ERSDCYCVCVERSRHRRLHFVLY------------------------------------- | 57 |
| 321 | ERSDCYCVCVERSRHRRLHFVLY------------------------------------- | 57 |
| 27 | ERSDCYCVCVERSRHRRLHF---------------------------------------- | 54 |
| 47 | ERSDCYCVCVERSRHRRLHFVLY------------------------------------- | 57 |
| 36 | ERSDCYCVCVERSRHRRLHFVLY------------------------------------- | 57 |
| 108 | ERSDCSCVCVERSRHRRLHFVLY------------------------------------- | 57 |
| 15 | ERSDCYCVCVERSRHRRLHFVLY------------------------------------- | 57 |
| 102 | ERSDCYCVCVERSRYRRLHFVLYLEKFFCLGMLLIYNLTPNPVLSETCAV---------- | 85 |
| 322 | ERSDCYCVCVERSRHRRLHFVL-------------------------------------- | 56 |
| 31 | ERSDCYCVCVERSRHRRLHFVLY------------------------------------- | 57 |
| PCAP3 | E----IRLSLCLCRKGRHKKLHFV--LY-------------------------------- | 56 |
| 172 | E----IRLSLCLCRKGRHKKLHFD--LY-------------------------------- | 56 |
| 302 | E----IRLSLCLCRKGRHKRLHFEKDLYSNNCFAEMLFICSFAPATLPQSLCPNLEFTKI | 92 |
| 303 | E----IRLSLCLCRKGRHKRLHFEKDLYSNNCFAEMLFICSFAPATLPQSLCPNLEFTKT | 92 |
| 296 | Q----IRLSLCLCRKGFHKRLHFEKDLYSNNCFAEMLFICSFAPATLPQSLCPNLEFTKT | 92 |
| CHR8_3rd | E----IRLSLCLCRKGRHKRLHFEKDLYSNYCFAEMLFICSFAPATLPQSLCPNLEFTKT | 92 |
| 208 | E----IRLSLCLCRKGRHKRLHF------------------------------------- | 53 |
| 212 | ERSDSYRLLLCLHRKGRHKRLH-------------------------------------- | 56 |
| 20 | ---REIRLLLCLCRK--------------------------------------------- | 51 |
| 32 | ---REIRLLLCLC----------------------------------------------- | 45 |
| 140 | ---RDIRLLLCLC----------------------------------------------- | 37 |
| 156 | ---REIRLLLCLC----------------------------------------------- | 43 |
| 282 | ---REIRLLLCLCRK--------------------------------------------- | 46 |
| 7 | GK-REIRLLLCLC----------------------------------------------- | 42 |
| 17 | ---REIRLLLCLCRK--------------------------------------------- | 45 |
| 201 | ---KEIRLLLCLCRK--------------------------------------------- | 45 |
| 254 | E----IRLLLCLC----------------------------------------------- | 43 |
| 312 | ------------------------------------------------------------ | |
| 239 | K----RDQIVTVSMQKRK------------------------------------------ | 47 |
| 319 | K----REQIVTVSV---------------------------------------------- | 43 |
| 242 | E----IR----------------------------------------------------- | 41 |
| 333 | K----RNQIVTVSV---------------------------------------------- | 43 |
| 213 | Q----------------------------------------------------------- | 34 |
| 190 | K----------------------------------------------------------- | 34 |
| 84 | DQ---------------------------------------------------------- | 36 |
| 149 | DQMLLCLCRK-------------------------------------------------- | 44 |
| 135 | DQ---------------------------------------------------------- | 35 |
| 226 | DQ---------------------------------------------------------- | 36 |
| 51 | DQ---------------------------------------------------------- | 36 |
| 71 | DQIVTVSVERSRHRRLHFVLY--------------------------------------- | 55 |
| 176 | DQ---------------------------------------------------------- | 36 |
| 261 | DQ---------------------------------------------------------- | 36 |
| 94 | DQ---------------------------------------------------------- | 36 |
| 233 | DQ---------------------------------------------------------- | 36 |
| 69 | ERSDCYCVCVERSRHRRFQKKK-------------------------------------- | 58 |
| 183 | EQ---------------------------------------------------------- | 36 |
| 268 | E----RSDCYCV------------------------------------------------ | 42 |
| 19 | ERSDCYCVCVERTRHRRFHFVLY------------------------------------- | 57 |
| 246 | ERSDCYCVCIERSRHRRHHFVLY------------------------------------- | 57 |
| 335 | ERSDFYCVCAERSRHRRHHFVLY------------------------------------- | 57 |
| 116 | ERSDCYCVCVERSRHRRHHFVLY------------------------------------- | 57 |
| 63 | ERSDCYCVCVERSRHRRHHFVLY------------------------------------- | 57 |
| 73 | ERSDCYHVCVERSRHRRHHFVLY------------------------------------- | 57 |
| 74 | ERSDCYCVCLERSRHRRLHFVL-------------------------------------- | 56 |
| 109 | ERTDCYCVCVERSRHRGLHFVLY------------------------------------- | 57 |
| 111 | ERTDCYCVCVERSRHRGLHFVLY------------------------------------- | 57 |
| 83 | ERTDCYCVCVERSRHRRLHFVLY------------------------------------- | 57 |
| 235 | ERSDCYCVCVERSRHRRHHFVLY------------------------------------- | 51 |
| 332 | ERSDCYCVCVERSRHRRLHFVLY------------------------------------- | 57 |
| 251 | ERSDCYCVCVERSRHRRLHFVLY------------------------------------- | 57 |
| 162 | ERSDCYCVCVERSRHRRLHFVLY------------------------------------- | 57 |
| 315 | ERSDCYCVCVERSRHRRLHFVLY------------------------------------- | 57 |
| 1 | ERSDCYCVCVERSRHRRLHFVLY------------------------------------- | 57 |
| 203 | ERSDCYCVCVERSRHRRLHFVLY------------------------------------- | 57 |
| 13 | ERSDCYCVCVERSRHRRLHFVLY------------------------------------- | 57 |
| 96 | ERSDCYCVCVERSRHRRHPFVLY------------------------------------- | 57 |
| 217 | ERSDCYCVCVERSRHRRLPFVLY------------------------------------- | 57 |
| 198 | GGSDFYSVCVERSRHRGPHFVLY------------------------------------- | 56 |
| 199 | ERSDCYCVCVERSRHRRLHFVLY------------------------------------- | 57 |
| 269 | ERSDCYCVCVERSRHRRLHFVLY------------------------------------- | 57 |
| 35 | ERSDCYCVCVERSRHRRLHFALY------------------------------------- | 57 |
| 24 | ERSDCYFVCVERSRHRRLHFVLY------------------------------------- | 57 |
| 113 | ERSDCYCVCVERSRHRRLHFVLYLEKFFCLGMLLIYNFTPNHVLSETC------------ | 83 |
| 130 | ERSDYYCVCVERSRHRRLHFVLY------------------------------------- | 57 |
| 318 | ERSDCYCVCVERSRHRRLHFVLY------------------------------------- | 57 |
| 29 | ERSDCYCVCVERSRHRRLHFVLY------------------------------------- | 57 |
| 232 | ERSDCYCVCVERSRHRRLHFVLY------------------------------------- | 57 |
| 80 | ERSDCYCVCVERSRHRRLHFVLY------------------------------------- | 57 |
| 160 | ERSDCYCVCVERSRHRRLHFVLH------------------------------------- | 57 |
| 68 | ERSNCYCVCVERSRHRRLHFVLY------------------------------------- | 57 |
| 249 | ERSDCYRVCVERSRHRRLHFVLY------------------------------------- | 57 |
| 231 | ERSDCYRVCVERSRHRRLHFVLY------------------------------------- | 57 |
| 234 | ERSDCYRVCVERSRHRRLHFVLY------------------------------------- | 57 |
| 42 | ERSDCYCVYVERSRHKRLHFVLY------------------------------------- | 55 |
| 236 | ERSDCYCVYVERSRHERLHFVLY------------------------------------- | 57 |
| 16 | ERSDCYCVCVERSRHRRLHFVLY------------------------------------- | 54 |
| 134 | ERSDCYCVCVERSRHRRLHFVLY------------------------------------- | 56 |
| 90 | ERSDCYCVCVERSRHRRLHFVLY------------------------------------- | 57 |
| 28 | ERSDCYCVCVERSRHRRLHFVLY------------------------------------- | 57 |
| 200 | ERSDCYCVCVES-RHRRLHEVLY------------------------------------- | 56 |
| 23 | ERSDCYCVCVERSRHRRLHFVLY------------------------------------- | 57 |
| 37 | ERSDCYCVCIERSRHRRLHFVLY------------------------------------- | 57 |
| 324 | ERTDCYCVYIERSRHRRLHFVLY------------------------------------- | 57 |
| 53 | ERSDCYCVCVERSWHRRLHFVLY------------------------------------- | 57 |
| 191 | ERSDCYCVCVERSRHRRLHFVLY------------------------------------- | 57 |
| 117 | ERSDCYCVCVERSRHRRLHFVLY------------------------------------- | 57 |
| 230 | ERSDCYCVCVERSRHRRLHFVLY------------------------------------- | 57 |
| 120 | ERSDCHCVCVERSRHRRLHFVLY------------------------------------- | 57 |
| 121 | ERSDCYCVCVERSRHRRLHFVLY------------------------------------- | 57 |
| 252 | ERSDCYCVCVERSRHRRLHFVLY------------------------------------- | 57 |
| 258 | ERSDLYCVCVERSRHRRLHFVLY------------------------------------- | 57 |
| 314 | ERSDCYCVCVERSRHRRLHFILY------------------------------------- | 57 |
| 323 | ERSDCYCVCVERSRHRRLHFVLY------------------------------------- | 57 |
| 131 | ERSDCYCVCVERSRHRRLHFVLY------------------------------------- | 57 |
| 326 | ERSDCYCVCVERRRHRRLHFVLY------------------------------------- | 57 |
| 151 | ERSDCYCVCVERSRHKRLHFVLY------------------------------------- | 57 |
| 248 | ERSDCYCVCVERSRHKRLHFVLY------------------------------------- | 57 |
| 75 | ERSDCYCVCVERSRHSRLHFVLY------------------------------------- | 57 |
| 56 | ERSDCYCVCVERSRHRRLHFVLY------------------------------------- | 46 |
| 311 | ERSDCYCVCIERSRHRRLHFVLY------------------------------------- | 48 |
| 166 | EISDCYCVYVERSRRKRLHFVV-------------------------------------- | 52 |
| 174 | EISDCYCVYVERSRRKRLHFVL-------------------------------------- | 52 |
| 155 | EISDCYCVYVERSRRKRLHFVLY------------------------------------- | 53 |
| 82 | EISDCYCVYVERSRHKRLHFVV-------------------------------------- | 52 |
| 245 | EGSDCYCVCVERSRHRRLHFVLH------------------------------------- | 43 |
| 310 | ERSECHCICVERSRHGRLHFVMY------------------------------------- | 71 |
| 87 | DKSDCCCVCVEGSRRRRLHFVLY------------------------------------- | 63 |
| 189 | ERSDCYCVCIERSRHRRLHFVLY------------------------------------- | 63 |
| 132 | ERSDCCCVCVEGGRRGRLHFVLY------------------------------------- | 55 |
| 317 | EKSDGCRVCVERGRHGR-FFILF------------------------------------- | 57 |
| 125 | ERSDCYCVCVERSRHRRLHFVLY------------------------------------- | 62 |
| 146 | QKSDCYCVCVERDRHRRLHFVLY------------------------------------- | 57 |
| 256 | ERSDCYCVCVERSKHRRLHFVLY------------------------------------- | 57 |
| 210 | KRSDCYCVCVERSRCRRLRFVLY------------------------------------- | 57 |
| 211 | KRSDCYCVCVERSRCRRLHFVLY------------------------------------- | 57 |
| 265 | KRSDCYCVCVERSRHGRLRFVLY------------------------------------- | 57 |
| 93 | ERSDCYCVCVERSRHRRLHFVLY------------------------------------- | 57 |
| 106 | ERSDCYCVCVERSRHRRLHFVLY------------------------------------- | 57 |
| 291 | ERSDCYCVCVERSRHKRLHFVLY------------------------------------- | 57 |
| 12 | ERSDCYCVCVERSRHRRLHFVLY------------------------------------- | 57 |
| 223 | ERSGCYCACVERSRHRRLHFVLY------------------------------------- | 57 |
| 86 | EKSDCYCVCVERSRHRRLHFVLY------------------------------------- | 57 |
| 88 | ERSDCYCVCVERSRHRRLHFVLY------------------------------------- | 57 |
| 205 | ERSDCYCVCVGRSRHRRLHFVLY------------------------------------- | 57 |
| 126 | ERSDCYCVCVERSIHRRLHFVLY------------------------------------- | 57 |
| 165 | ERSDCYCVCVERNRHRRLHFVLY------------------------------------- | 57 |
| 330 | ERSDCYCVCVERSRHRRLHFVLY------------------------------------- | 57 |
| 247 | ERSDCYCVCVERSRHRRLHFVLY------------------------------------- | 57 |
| 4 | ERSDCYCVCVERSRHRILHFVLY------------------------------------- | 57 |
| 10 | ERSDCYCVCVERSRHRILHFVLY------------------------------------- | 57 |
| 81 | ERSDCYCVCVERSRHRRLHFLLY------------------------------------- | 57 |
| 115 | ERSDCYCVCVERSRHRRLHFLLY------------------------------------- | 57 |
| 175 | ERSDCYCVCVERSRHRRLHFL--------------------------------------- | 55 |
| 275 | ERSDCYCVCVERSRHRRLHFVL-------------------------------------- | 56 |
| 270 | ERSDCYCVYVERSRHRRLHFVLY------------------------------------- | 57 |
| chrY_3rd | ERSDCYCVYVEKKDIRDSILKKTCTLNNCFAEMLLICSFAPATLTQPGAHKNMCCMESRL | 96 |
| 272 | ERSDCYCVYVEREDIRDSILKKTCTLNNCFAQMLLICSFAPATLTQPGAHKNMCCMKSRF | 96 |
| 308 | ERSDCYCVYVEREDIRNSIL--TCTLNNCFAEMLLICNFAPATLPQ-------------- | 80 |
| 40 | EISDCYCIFVEKEDIRNSIL--TCTVNNCFA----------------------------- | 65 |
| 170 | ERSHCYCVYVEKEDI-NSILS--CTKKNYFA----------------------------- | 65 |
| 266 | ERSDCYCVCVEREDIRNSILT--CTLNNWLAEMLLICDFAPNLSSQ-------------- | 76 |
| 5 | ERSDGYCVYVEKEDIRNFILI--CTLNNCFA----------------------------- | 65 |
| 64 | ERSDCHCAYVEREDIRDSILKKTCTLNNCFAEMLLICSF--------------------- | 75 |
| 221 | VRSDCYCIYVER------------------------------------------------ | 46 |
| 43 | ERSDCYCVYIEREDIRDSILKKICTLSNCFAEMLLICSFAPATLPQP------------- | 83 |
| 50 | ERSDCYCVYIEREDIRDSILKKNCTLNNCFAEMFLICSFAPATFPQP------------- | 83 |
| 60 | ERSDCYCVYIEREDIRDSILEKTCTLNNCFAEMLLICSFAPATLPQP------------- | 83 |
| 192 | ERSDCYCVYTEREDIRDSILKKTCTLNNCFAEMLLICSFAPATLP--------------- | 81 |
| 57 | ERSDCYCVYIER------------------------------------------------ | 46 |
| 187 | ERSDCYCVYIEREDIRDSILKKTCTLNSCFDRDSCLSAFMCLLLPQ-------------- | 74 |
| 293 | ERSDCYCVYIEREAIR-------------------------------------------- | 50 |
| 299 | ERSDCYCVYIEREAIRDSILKKTCTLNNCLLRCCLSVALPQPLCPN-------------- | 82 |
| 292 | ERSDSYCVYIER------------------------------------------------ | 46 |
| 207 | ERSDSYCVYIER------------------------------------------------ | 46 |
| 178 | ERSDCYCVYIER------------------------------------------------ | 46 |
| 152 | ERSDCYCVYVER------------------------------------------------ | 46 |
| 195 | EGSDCHCVYAER------------------------------------------------ | 46 |
| 138 | ERSDCYCVYVEKEDIRNSILICIKKNCSALRC---------------------------- | 68 |
| 204 | ERSDCHSVYVEK------------------------------------------------ | 46 |
| 22 | ERSDCYCVYVEK------------------------------------------------ | 46 |
| 11 | ERSDCYCVYVEK------------------------------------------------ | 46 |
| 302 | CVV-------- | 95 |
| 303 | CVV-------- | 95 |
| 296 | CVV-------- | 95 |
| CHR8_3rd | CVV-------- | 95 |
| chrY_3rd | KGSRAVQDVPC | 107 |
| 272 | KGSRAVQDVPC | 107 |

All publications and patent applications mentioned in this specification are incorporated herein by reference to the same extent as if each individual publication or patent application were specifically and individually indicated to be incorporated by reference.

The foregoing description of preferred embodiments of the invention has been presented by way of illustration and example for purposes of clarity and understanding. It is not intended to be exhaustive or to limit the invention to the precise forms disclosed. It will be readily apparent to those of ordinary skill in the art in light of the teachings of this invention that many changes and modifications may be made thereto without departing from the spirit of the invention. It is intended that the scope of the invention be defined by the appended claims and their equivalents.

TABLE 1
DESCRIPTION OF SPLICE VARIANTS
Consensus sequences of splice forms A to J (FIG. 17) are given as SEQ IDs 18-27. These
represent consensus sequences of all cDNA clones characterized and in general represent the
sequence variability observed in the genomic alignments of FIG. 19-22.

| SPLICE | | |
|---|---|---|
| FORM | | PROTEIN |
| (SEQ ID) | DESCRIPTION | SEQ ID |
| A | Splice variant that joins exons 1 (Splice Site I) and 3 (Splice Site XIII), without | — |
| 18 | exon 2. Probably does not encode any protein, as its only methionines are from | |
| | the ORFs in exon 3. This splice form was detected in prostate cancer cell lines | |
| | LNCaP and PC3. By agarose gel analysis, a product corresponding to this | |
| | splice form was detected in all cell line and tumor samples analyzed. | |
| B | PCAP2 Splice variant that joins exon 1 (Splice Site I) to exon 2 (Splice Site IV | 28 |
| 19 | to VI) and to exon 3 (Splice Site XIII). Derived from a type I PCAV. Encodes | |
| | a 74aa protein from the env start codon and ends in the +2 frame termination | |
| | codon in exon 3. Detected in all prostate cancer cell lines tested and in tissue | |
| | samples from prostate tumors. | |
| C | PCAP2 Splice variant that joins exon 1 (Splice Site I) to exon 2 (Splice Site V | 29 |
| 20 | to VI) and to exon 3 (Splice Site XIII). Derived from a type I PCAV. Encodes | |
| | a 74aa protein identical to that encoded by Splice Form B, from the env start | |
| | codon to the +2 frame termination codon in exon 3. Detected in all prostate | |
| | cancer cell lines tested and in tissue samples from prostate cancer tumors. | |
| D | PCAP2 Splice variant that joins exon 1 (Splice Site I) to exon 1.5 (Splice Site | 30 |
| 21 | II and III), to exon 2 (Splice Site V to VI) and to exon 3 (Splice Site XIII). | 31 |
| | Derived from a type I PCAV. Exon 1.5 contains a potential initiation | |
| | methionine and so this splice form potentially encodes an additional | |
| | polypeptide different from the one initiated by the env methionine in exon 2. It | |
| | also encode for the 74aa protein identical to that encoded by Splice Form B, | |
| | from the env start codon to the +2 frame termination codon in exon 3. Detected | |
| | in prostate cancer cell line MDA Pca-2b. | |
| E | PCAP1 Splice variant that joins exon 1 (Splice Site I) to exon 2 (Splice Site V | 32 |
| 22 | to VIII) and to exon 3 (Splice Site XIII). Derived from a type II PCAV. | |
| | Encodes a 76aa protein from the env start codon to the cORF termination | |
| | codon in exon 3. Detected in most prostate cancer cell lines tested and in tissue | |
| | samples from prostate cancer tumors. | |
| F | Splice variant that joins exon 1 (Splice Site I) to exon 2 (Splice Site V to IX) | 33 |
| 23 | and to exon 3 (Splice Site XIII). Derived from a type II PCAV. Encodes a 95aa | |
| | protein from the env start codon to the cORF termination codon in exon 3. | |
| | Detected in most prostate cancer cell lines tested and in the tissue samples from | |
| | prostate cancer tumors. | |
| G | Splice variant that joins exon 1 (Splice Site I) to exon 2 (Splice Site V to XI) | 34 |
| 24 | and to exon 3 (Splice Site XIII). Derived from a type I PCAV. If the env start | |
| | codon is used, the ORF is very short and ends with a TAA termination codon | |
| | at position 6922 in the type I PCAV (FIG. 21). However, a second potential | |
| | initiation methionine is found at position 6918 in the type I PCAV (FIG. 21), | |
| | and this ORF encodes a 127aa protein that ends at the +2 frame termination | |
| | codon in exon 3. Detected in most prostate cancer cell lines tested and in the | |
| | tissue samples from prostate cancer tumors. | |
| H | Splice variant that joins exon 1 (Splice Site I) to exon 2 (Splice Site V to X) | 35 |
| 25 | and to exon 3 (Splice Site XIII). Derived from a type I PCAV. As for splice | |
| | form G, if the env start codon is used then the ORF is very short. Using the | |
| | second initiation methionine at position 6918 in the type I PCAV (FIG. 21), | |
| | however, this ORF encodes a 105aa protein that ends at the +2 frame | |
| | termination codon in exon 3. Detected in most prostate cancer cell lines tested | |
| | and in the tissue samples from prostate cancer tumors. | |
| I | PCAP3 Splice variant that joins exon 1 (Splice Site I) to exon 2 (Splice Site V | 36 |
| 26 | to VII) and to exon 3 (Splice Site XIII). Derived from a type II PCAV. | |
| | Encodes a 79aa protein from the env start codon and ends at the +2 frame | |
| | termination codon in exon 3. Detected in prostate cancer cell line MDA Pca- | |
| | 2b. | |
| J | This cDNA clone was identified using a PCR forward primer in exon 2 and a | 37 |
| 27 | reverse primer in exon 3. The mRNA from which this splice form is derived | |
| | probably does not have intron 1 spliced out. Splicing of intron 2 was between | |
| | Splice Sites XII & XIII. | |
| K | Created by joining exon 2 at ‘Potential Splice Site A’ (FIG. 21) with exon 3 at | 39 |
| 38 | Splice Site XIII. Encodes PCAP4. | |
| L | Created by joining exon 2 at Splice Site VIII (FIG. 21) with exon 3 at | 41 |
| 40 | ‘Potential Splice Site B’. Encodes PCAP4a. | |
| M | Created by joining exon 2 at Splice Site IX (FIG. 21) with exon 3 at ‘Potential | 43 |
| 42 | Splice Site B’. Encodes PCAP5. | |

TABLE 2
SEQUENCE LISTING

| SEQ ID | DESCRIPTION |
|---|---|
| 1 | U5 region of herv-k(hml-2.hom) {GenBank AF074086} |
| 2 | U3 region of herv-k(hml-2.hom) |
| 3 | R region of herv-k(hml-2.hom) |
| 4 | RU5 region of herv-k(hml-2.hom) |
| 5 | U3R region of herv-k(hml-2.hom) |
| 6 | Non-coding region between U5 and first 5′ splice site of |
| | herv-k(hml-2.hom) |
| 7 | PCAP4 |
| 8 | PCAP4a |
| 9 | PCAP5 |
| 10 | PCAP1 |
| 11 | PCAP2 |
| 12 | PCAP3 |
| 13 | cORF |
| 14 | TAR |
| 15 | FIG. 15, forward primer |
| 16 | FIG. 15, reverse primer 1 |
| 17 | FIG. 15, reverse primer 2 |
| 18 | Splice form A |
| 19 | Splice form B |
| 20 | Splice form C |
| 21 | Splice form D |
| 22 | Splice form E |
| 23 | Splice form F |
| 24 | Splice form G |
| 25 | Splice form H |
| 26 | Splice form I |
| 27 | Splice form J |
| 28 | Splice form A protein |
| 29 | Splice form B protein |
| 30 | Splice form C protein |
| 31 | Splice form D protein |
| 32 | Splice form E protein |
| 33 | Splice form F protein |
| 34 | Splice form G protein |
| 35 | Splice form H protein |
| 36 | Splice form I protein |
| 37 | Splice form J protein |
| 38 | Splice form K |
| 39 | Splice form K protein |
| 40 | Splice form L |
| 41 | Splice form L protein |
| 42 | Splice form M |
| 43 | Splice form M protein |
| 44 | LTR of herv-k(hml-2.hom) |
| 45 | HML-2 LTR |
| 46 | HML-2 LTR |
| 47 | HML-2 LTR |
| 48 | HML-2 LTR |
| 49 | Putative TAR of herv-k(hml-2.hom) |
| 50 | Remainder of herv-k(hml-2.hom) LTR |
| 51 | Region downstream of ‘potential splice site B’ in FIG. 22 |
| 52-59 | Oligos and primers used in mapping 5′ end of mRNA |
| 60 | V5 tag |
| 61-67 | Motifs common to exon 3 of various PCAPs |
| 68 | Exon 3 region of PCAP3 |
| 69-76 | Optional disclaimed amino acid sequences |
| 77 | MDALTR |
| 78-477 | Third frame sequences from human genome |
| 478 | SEQ ID 49, excluding its 77 5′ nucleotides |

### REFERENCES

### The Contents of which are Hereby Incorporated in Full by Reference

{1} International patent application WO02/46477 (PCT/US01/47824. filed Dec. 7, 2001).
{2} U.S. patent application Ser. No. 10/016,604 (filed Dec. 7, 2001).
{3} Magin-Lachmann (2001) J. Virol. 75(21):10359-71.
{4} Löwer et al. (1995) J. Virol. 69:141-149.
{5} Magin et al. (1999) J. Virol. 73:9496-9507.
{6} GenBank entry Y17832.
{7} Mayer et al. (1999) Nat. Genet. 21 (3), 257-258 (1999)
{8} Farrell (1998) RNA Methodologies (Academic Press; ISBN 0-12-249695-7).
{9} Robbins et al. (1997) Clin Lab Sci 10(5):265-71.
{10} Ylikoski et al. (1999) Clin Chem 45(9):1397-407
{11} Ylikoski et al. (2001) Biotechniques 30:832-840
{12} Shirahata & Pegg (1986) J. Biol. Chem. 261(29):13833-7.
{13} Suzuki et al. (2001) Br J Cancer 85:1731-1737.
{14} Revillion et al. (2000) Eur J Cancer 36:1038-1042.
{15} Miyakis et al. (1998) Biochem Biophys Res Commun 251:609-612.
{16} Berois et al. (1997) Anticancer Res 17(4A):2639-2646.
{17} Sambrook et al. (1989) Molecular Cloning: A Laboratory Manual (New York, Cold Spring Harbor Laboratory)
{18} Short protocols in molecular biology (4th edition, 1999) Ausubel et al. eds. ISBN 0-471-32938-X.
{19} U.S. Pat. No. 5,707,829
{20} Current Protocols in Molecular Biology (F. M. Ausubel et al., eds., 1987) Supplement 30.
{21} EP-B-0509612
{22} EP-B-0505012
{23} Hashido et al. (1992) Biochem. Biophys. Res. Comm. 187:1241-1248.
{24} Geysen et al. (1984) PNAS USA 81:3998-4002.
{25} Carter (1994) Methods Mol Biol 36:207-23.
{26} Jameson, B A et al., 1988, CABIOS 4(1):181-186.
{27} Raddrizzani & Hammer (2000) Brief Bioinform 1(2):179-89.
{28} De Lalla et al. (1999) J. Immunol. 163:1725-29.
{29} Brusic et al. (1998) Bioinformatics 14(2):121-30
{30} Meister et al. (1995) Vaccine 13(6):581-91.
{31} Roberts et al. (1996) AIDS Res Hum Retroviruses 12(7):593-610.
{32} Maksyutov & Zagrebelnaya (1993) Comput Appl Biosci 9(3):291-7.
{33} Feller & de la Cruz (1991) Nature 349(6311):720-1.
{34} Hopp (1993) Peptide Research 6:183-190.
{35} Welling et al. (1985) FEBS Lett. 188:215-218.
{36} Davenport et al. (1995) Immunogenetics 42:392-297.
{37} Morris et al. (2001) Vaccine 20(1-2):12-15.
{38} Goldstein et al. (2001) Vaccine 19(13-14):1738-46
{39} Tosi et al. (2000) Eur. J. Immunol. 30(4):1120-6
{40} Tahtinen et al. (1997) Biomed Pharmacother 51(10):480-7.
{41} Nath et al. (1996) J. Virol. 70:1475-1480.
{42} Pardi et al. (1995) J Infect Dis 172:554-557.
{43} Lairmore et al. (1995) Biomed Pept Proteins Nucleic Acids 1:117-122.
{44} Sakakibara et al. (1998) J Vet Med Sci 60:599-605.
{45} Smith and Waterman, Adv. Appl. Math. (1981) 2: 482-489.
{46} Go et al., Int. J. Peptide Protein Res. (1980) 15:211
{47} Querol et al., Prot. Eng. (1996) 9:265
{48} Olsen and Thomsen, J. Gen. Microbiol. (1991) 137:579
{49} Clarke et al., Biochemistry (1993) 32:4322
{50} Wakarchuk et al., Protein Eng. (1994) 7:1379
{51} Toma et al., Biochemistry (1991) 30:97
{52} Haezerbrouck et al., Protein Eng. (1993) 6:643
{53} Masul et al., Appl. Env. Microbiol. (1994) 60:3579
{54} U.S. Pat. No. 4,959,314
{55} Breedveld (2000) Lancet 355(9205):735-740.
{56} Gorman & Clark (1990) Semin. Immunol. 2:457-466
{57} Jones et al., Nature 321:522-525 (1986)
{58} Morrison et al., Proc. Natl. Acad. Sci, USA., 81:6851-6855 (1984)
{59} Morrison and Oi, Adv. Immunol., 44:65-92 (1988)
{60} Verhoeyer et al., Science 239:1534-1536 (1988)
{61} Padlan, Molec. Immun. 28:489-498 (1991)
{62} Padlan, Molec. Immunol. 31(3):169-217 (1994).
{63} Kettleborough, C. A. et al., Protein Eng. 4(7):773-83 (1991).
{64} Chothia et al., J. Mol. Biol. 196:901-917 (1987)
{65} Kabat et al., U.S. Dept. of Health and Human Services NIH Publication No. 91-3242 (1991)
{66} U.S. Pat. No. 5,530,101.
{67} U.S. Pat. No. 5,585,089.
{68} WO 98/24893
{69} WO 91/10741
{70} WO 96/30498
{71} WO 94/02602
{72} U.S. Pat. No. 5,939,598.
{73} WO 96/33735
{74} Johnston et al. (2001) Ann Neurol 50(4):434-42.
{75} Medstrand et al. (1998) J Virol 72(12):9782-7.
{76} Lisziewicz et al. (1993) PNAS USA 90:8000-8004
{77} Chang et al. (1994) Gene Ther 1(3):208-16.
{78} Fraisier et al. (1998) Gene Ther 5(7):946-54.
{79} Fraisier et al. (1998) Gene Ther 5(12):1665-1676.
{80} Wyszko et al. (2001) Int J Biol Macromol 28(5):373-80.
{81} Rao et al. (2000) J. Biomolec. Struc. Dynam. 11(1).
{82} Tamilarasu et al. (2000) Bioorg Med Chem Lett 10(9):971-4.
{83} Hamy et al. (1998) Biochemistry 37(15):5086-95
{84} Yamamoto et al. (2000) Genes Cells 5:371-388
{85} Coburn & Cullen (2002) J. Virol. 76:9225-9231.
{86} Cantor et al. (1993) Proc Natl Acad Sci USA 90:10932-10936.
{87} Miyano-Kurosald et al. (1996) Virus Genes 12:205-217.
{88} Cantor & Palmer (1992) Antisense Res Dev 2:147-152.
{89} Radrizzani et al. (1999) Medicina (B Aires) 59(6):753-8
{90} Bianchini et al. (2001) J Immunol Methods 252(1-2):191-197
{91} Zamore (2001) Nat Struct Biol 8:746-750.
{92} Carthew (2001) Curr Opin Cell Biol 13:244-248.
{93} Billy et al. (2001) PNAS USA 98:14428-14433.
{94} Yang et al. (2001) Mol Cell Biol 21:7807-7816.
{95} Carmichael (2002) Nature 418:379-380.
{96} Xia et al. (2002) Nature Biotech 20:1006-1010.
{97} Mei et al. (1998) Biochemistry 37:14204-12.
{98} An et al. (1998) Bioorg Med Chem Lett 8(17):2345-50
{99} McSharry (1999) Antiviral Res 43(1):1-21.
{100} Kuhelj et al. (2001) J Biol Chem 276(20):16674-82.
{101} Schommer et al. (1996) J Gen Virol 77:375-379.
{102} Magin et al. (2000) Virology 274:11-16.
{103} Boese et al. (2001) FEBS Lett 493(2-3):117-21.
{104} Further details: Rational drug design: novel methodology and practical applications, ACS Symposium Series vol. 719 (Parrill & Reddy eds., 1991).
{105} Available from Tripos Inc (http://www.tripos.com).
{106} Available from Oxford Molecular (http://www.oxmol.co.uk/).
{107} Available from Molecular Simulations Inc (http://www.msi.com/).
{108} Available from Hypercube Inc (http://www.hyper.com/).
{109} Available from Pyramid Learning (http://www.chemsite.org/).
{110} Filikov et al. (2000) J Comput Aided Mol Des 14(6):593-610
{111} Re et al. (2001) New Microbiol 24(2):197-205.
{112} Ensoli & Cafaro (2000) Peptides 21:1839-1847.
{113} Ensoli & Cafaro (2000) J Biol Regul Homeost Agents 14(1):22-6
{114} Boykin et al. (2000) Peptides 21:1839-1847.
{115} Dezzutti et al. (1990) J Med Primatol 19:305-316.
{116} Cafaro et al. (2001) Vaccine 19(20-22):2862-77
{117} Ohashi et al. (2000) J Virol 74:9610-9616.
{118} WO90/14837
{119} Vaccine Design—the subunit and adjuvant approach (1995) ed. Powell & Newman
{120} WO00/07621
{121} GB-2220221
{122} EP-A-0689454
{123} EP-A-0835318
{124} EP-A-0735898
{125} EP-A-0761231
{126} WO99/52549
{127} WO01/21207
{128} WO01/21152
{129} WO00/62800
{130} WO00/23105
{131} WO99/11241
{132} WO98/57659
{133} WO93/13202.
{134} Gennaro (2000) Remington: The Science and Practice of Pharmacy. 20th edition, ISBN: 0683306472.
{135} WO 93/14778
{136} Findeis et al., Trends Biotechnol. (1993) 11:202
{137} Chiou et al., Gene Therapeutics: Methods And Applications Of Direct Gene Transfer (J. A. Wolff, ed.) (1994)
{138} Wu et al., J. Biol. Chem. (1988) 263:621
{139} Wu et al., J. Biol. Chem. (1994) 269:542
{140} Zenke et al., Proc. Natl. Acad. Sci. (USA) (1990) 87:3655
{141} Wu et al., J. Biol. Chem. (1991) 266:338
{142} Jolly, Cancer Gene Therapy (1994) 1:51
{143} Kimura, Human Gene Therapy (1994) 5:845
{144} Connelly, Human Gene Therapy (1995) 1:185
{145} Kaplitt, Nature Genetics (1994) 6:148
{146} WO 90/07936
{147} WO 94/03622
{148} WO 93/25698
{149} WO 93/25234
{150} U.S. Pat. No. 5,219,740
{151} WO 93/11230
{152} WO 93/10218
{153} U.S. Pat. No. 4,777,127
{154} GB Patent No. 2,200,651
{155} EP-A-0 345 242
{156} WO 91/02805
{157} WO 94/12649
{158} WO 93/03769
{159} WO 93/19191
{160} WO 94/28938
{161} WO 95/11984
{162} WO 95/00655
{163} Curiel, Hum. Gene Ther. (1992) 3:147
{164} Wu, J. Biol. Chem. (1989) 264:16985
{165} U.S. Pat. No. 5,814,482
{166} WO 95/07994
{167} WO 96/17072
{168} WO 95/30763
{169} WO 97/42338
{170} WO 90/11092
{171} U.S. Pat. No. 5,580,859
{172} U.S. Pat. No. 5,422,120
{173} WO 95/13796
{174} WO 94/23697
{175} WO 91/14445
{176} EP 0524968
{177} Philip, Mol. Cell Biol. (1994) 14:2411
{178} Woffendin, Proc. Natl. Acad. Sci. (1994) 91:11581
{179} U.S. Pat. No. 5,206,152
{180} WO 92/11033
{181} U.S. Pat. No. 5,149,655
{182} WO 92/11033
{183} Larsson, E., et al., Current Topics in Microbiology and Immunology 148:115 (1989)
{184} Mariani-Costantini, et al., J. Virol, 63:4982 (1989) and Shill, et at, Virology 182:495 (1991)
{185} Tönjes et al. (1996) J. AIDS Hum. Retrovir. 13(Suppl 1):S261-S267.
{186} Barbulescu et al., Curr. Biol. 9:861 (1999)
{187} Ono, et al., J. Virol. 58:937 (1986)
{188} Löwer et al., Proc. Natl. Acad. Sci USA 90:4480 (1993)
{189} Ono et al., (1986) J. Virol. 60:589
{190} Boller, et al., Virol. 196:349 (1993)
{191} Yang et al., Proc. Natl. Acad. Sci USA 96:13404 (1999)
{192} Mueller-Lantzsch et al., AIDS Research and Human Retroviruses 9:343-350 (1993)
{193} Herbst et al., Amer. J. Pathol. 149:1727 (1996)
{194} U.S. Pat. No. 5,858,723
{195} Löwer et al., Proc. Natl. Acad. Sci USA 93:5177 (1996)
{196} Löwer et al., Virology 192:501 (1993)
{197} Blomberg et al., J. Cancer. res. Clin. Oncol. 121: Supp. 1, 3 (1995)
{198} Genbank accession number AB047240
{199} Andersson et al. (1999) J. Gen. Virol. 80:255-260.
{200} Zsiros et al. (1998) J Gen. Virol. 79:61-70.
{201} Tönjes et al. (1999) J. Virol. 73:9187-9195.
{202} Yoshida (X) Annual Review of Immunology 19:475-496.
{203} Bello et al. (1997) Carcinogenesis 18:1215-1223.
{204} Armbruester et al. (2002) Clinical Cancer Research 8:1800-1807.
{205} Sedivy (1998) Proc Natl Acad Sci USA 95:9078-9081.
{206} Hahn et al. (2002) Mol Cell Biol. 22(7):2111-2123.
{207} Hahn et al. (1999) Nature 400(6743):464-468.
{208} De Marzo et al. (1998) J Urol. 160:2381-2392.
{209} Sakr & Partin (2001) Urology 57(4 Suppl 1):115-120.

1.-19. (canceled)

20. An isolated polynucleotide comprising:
(a) the nucleotide sequence selected from the group consisting of SEQ ID NOs: 19, 20, 21, 24, 25, 26, 38, 40, and 42;
(b) a fragment of at least 7 nucleotides of the nucleotide sequence of SEQ ID NO: 19, 20, 21, 24, 25, 38 or 40;
(c) a nucleotide sequence having at least 90% identity to the nucleotide sequence of SEQ ID NO: 19, 20, 21, 24, 25, 38 or 40; or
(d) the complement of (a), (b) or (c),
wherein the polynucleotide or its complement encodes an expression product produced by a splicing event in which the 5′ region and start codon of the env coding region of a HML-2 are joined to a downstream coding region in the reading frame +2 relative to that of env.

21. An isolated polynucleotide comprising:
(a) the nucleotide sequence encoding a polypeptide having an amino acid sequence selected from the group consisting of SEQ ID NOs: 7, 8, 9, 10, 11, 12, 28, 29, 30, 31, 34, 35, 39, 41, 43, 66 to 67, 68 and 78 to 277;
(b) a fragment of at least 7 nucleotides of the nucleotide sequence of (a);
(c) a nucleotide sequence having at least 90% identity to the nucleotide sequence of (a); or
(d) the complement of (a), (b) or (c),
wherein the polynucleotide or its complement encodes an expression product produced by a splicing event in which the 5′ region and start codon of the env coding region of a HML-2 are joined to a downstream coding region in the reading frame +2 relative to that of env.

22. An isolated polynucleotide encoding a polypeptide having an amino acid sequence having at least 90% identity to an amino acid sequence selected from the group consisting of SEQ ID NOs: 7, 8, 9, 10, 11, 12, 28, 29, 30, 31, 34, 35, 39, 41, 43, 66 to 67, 68 and 78 to 277, wherein the polypeptide is an expression product produced by a splicing event in which the 5′ region and start codon of the env coding region of a HML-2 are joined to a downstream coding region in the reading frame +2 relative to that of env.

23. The isolated polynucleotide of claim 22, wherein the polynucleotide encodes a polypeptide having at least 90% identity to an amino acid sequence selected from the group consisting of SEQ ID NOs: 7, 8, 9, 10, 11, and 12.

24. The isolated polynucleotide of claim 22, wherein the polynucleotide encodes a polypeptide having at least 90% identity an amino acid sequence selected from the group consisting of SEQ ID NOs: 28, 29, 30, 31, 34, 35, 39, 41, and 43.

25. The isolated polynucleotide of claim 22, wherein the polynucleotide encodes a polypeptide having an amino acid sequence selected from the group consisting of SEQ ID NOs: 66 to 67, and 68.

26. The isolated polynucleotide of claim 22, wherein the polynucleotide encodes a polypeptide having at least 90% identity an amino acid sequence selected from the group consisting of SEQ ID NOs: 78 to 277.

27. An isolated polynucleotide having a nucleotide sequence with at least 90% identity to the nucleotide sequence selected from the group consisting of SEQ ID NOs: 19, 20, 21, 24, 25, 26, 38, 40, and 42; wherein the polynucleotide encodes an expression product produced by a splicing event in which the 5′ region and start codon of the env coding region of a HML-2 are joined to a downstream coding region in the reading frame +2 relative to that of env.

28. The isolated polynucleotide of claim 27, wherein the polynucleotide has a nucleotide sequence selected from the group consisting of SEQ ID NOs: 19, 20, 21, 24, 25, 26, 38, 40, and 42.

29. A method for preventing or treating cancer, the method comprising a step of administering a composition comprising the polynucleotide of claim 20.

30. The method of claim 29, wherein the cancer is prostate cancer.

31. A method for preventing or treating cancer, the method comprising a step of administering a composition comprising the polynucleotide of claim 21.

32. The method of claim 31, wherein the cancer is prostate cancer.


SEQUENCE LISTING (478 sequences)

SEQ ID NO: 1 - DNA, 89 bp, HERV-K
ctttgtctctgtgtctttttcttttccaaatctctcgtcccaccttacgagaaacacccacaggtgtgtaggggcaacccacccctaca

SEQ ID NO: 2 - DNA, 560 bp, HERV-K
tgtggggaaaagcaagagagatcagattgttactgtgtctgtgtagaaagaagtagacataggagactccattttgttatgtactaagaaaaattcttctgccttgagattctgttaatctatgaccttacccccaaccccgtgctctctgaaacatgtgctgtgtccactcagggttaaatggattaagggcggtgcaggatgtgctttgttaaacagatgcttgaaggcagcatgctccttaagagtcatcaccactccctaatctcaagtacccagggacacaaaaactgcggaaggccgcagggacctctgcctaggaaagccaggtattgtccaacgtttctccccatgtgatagcctgaaatatggcctcgtgggaagggaaagacctgaccgtcccccagcccgacacccgtaaagggtctgtgctgaggaggattagtaaaagaggaaggaatgcctcttgcagttgagacaagaggaaggcatctgtctcctgcctgtccctgggcaatggaatgtctcggtataaaacccgattgtatgctccatctact

SEQ ID NO: 3 - DNA, 319 bp, HERV-K
gagatagggaaaaaccgccttagggctggaggtgggacctgcgggcagcaatactgctttgtaaagcactgagatgtttatgtgtatgcatatctaaaagcacagcacttaatcctttacattgtctatgatgcaaagacctttgttcacatgtttgtctgctgaccctctccccacaattgtcttgtgaccctgacacatccccctcttcgagaaacacccacagatgatcagtaaatactaagggaactcagaggctggcgggatcctccatatgctgaacgctggttccccgggtccccttctttctttctctata

SEQ ID NO: 4 - DNA, 408 bp, HERV-K
gagatagggaaaaaccgccttagggctggaggtgggacctgcgggcagcaatactgctttgtaaagcactgagatgtttatgtgtatgcatatctaaaagcacagcacttaatcctttacattgtctatgatgcaaagacctttgttcacatgtttgtctgctgaccctctccccacaattgtcttgtgaccctgacacatccccctcttcgagaaacacccacagatgatcagtaaatactaagggaactcagaggctggcgggatcctccatatgctgaacgctggttccccgggtccccttctttctttctctatactttgtctctgtgtctttttcttttccaaatctctcgtcccaccttacgagaaacacccacaggtgtgtaggggcaacccacccctaca

SEQ ID NO: 5 - DNA, 879 bp, HERV-K
tgtggggaaaagcaagagagatcagattgttactgtgtctgtgtagaaagaagtagacataggagactccattttgttatgtactaagaaaaattcttctgccttgagattctgttaatctatgaccttacccccaaccccgtgctctctgaaacatgtgctgtgtccactcagggttaaatggattaagggcggtgcaggatgtgctttgttaaacagatgcttgaaggcagcatgctccttaagagtcatcaccactccctaatctcaagtacccagggacacaaaaactgcggaaggccgcagggacctctgcctaggaaagccaggtattgtccaacgtttctccccatgtgatagcctgaaatatggcctcgtgggaagggaaagacctgaccgtcccccagcccgacacccgtaaagggtctgtgctgaggaggattagtaaaagaggaaggaatgcctcttgcagttgagacaagaggaaggcatctgtctcctgcctgtccctgggcaatggaatgtctcggtataaaacccgattgtatgctccatctactgagatagggaaaaaccgccttagggctggaggtgggacctgcgggcagcaatactgctttgtaaagcactgagatgtttatgtgtatgcatatctaaaagcacagcacttaatcctttacattgtctatgatgcaaagacctttgttcacatgtttgtctgctgaccctctccccacaattgtcttgtgaccctgacacatccccctcttcgagaaacacccacagatgatcagtaaatactaagggaactcagaggctggcgggatcctccatatgctgaacgctggttccccgggtccccttctttctttctctata

SEQ ID NO: 6 - DNA, 108 bp, HERV-K
tctggtgcccaacgtggaggcttttctctagggtgaaggtacgctcgagcgtggtcattgaggacaagtcgacgagagatcccgagtacatctacagtcagccttacg

SEQ ID NO: 7 - protein, 129 aa, HERV-K
MNPSEMQRKAPPRRRRHRNRAPLTHKMNKMVTSEEQMKLPSTKKAEPPTWAQLKKLTQLATKYLENTKVILQVYPTAPKRQRPSRTGHDDDGGFVEKKRGKCGEKQERSDCYCVCVERSRHRRLHFVLY

SEQ ID NO: 8 - protein, 125 aa, HERV-K
MNPSEMQRKAPPRRRRHRNRAPLTHKMNKMVTSEEQMKLPSTKKAEPPTWAQLKKLTQLATKYLENTKVYPTAPKRQRPSRTGHDDDGGFVEKKRGKCGEKQERSDCYCVCVERSRHRRLHFVLY

SEQ ID NO: 9 - protein, 144 aa, HERV-K
MNPSEMQRKAPPRRRRHRNRAPLTHKMNKMVTSEEQMKLPSTKKAEPPTWAQLKKLTQLATKYLENTKVTQTPESMLLAALMIVSMVVYPTAPKRQRPSRTGHDDDGGFVEKKRGKCGEKQERSDCYCVCVERSRHRRLHFVLY

SEQ ID NO: 10 - protein, 86 aa, HERV-K
MNPSEMQRKAPPRRRRHRNRAPLTHKMNKMVTSEEQMKLPSTKKAEPPTWAQLKKLTQLATKYLENTKSAGVPNSSEETATIENGP

SEQ ID NO: 11 - protein, 74 aa, HERV-K
MNPSEMQRKGPPQRCLQVYPTAPKRQRPSRTGHDDDGGFVEKKRGKCGEKQERSDCYCVCVERSRHRRLHFVLY

SEQ ID NO: 12 - protein, 79 aa, HERV-K [sequence omitted]

SEQ ID NO: 13 - protein, 105 aa, HERV-K
MNPSEMQRKAPPRRRRHRNRAPLTHKMNKMVTSEEQMKLPSTKKAGPPTWAQLKKLTQLATKYLENTKVTQTPESMLLAALMIVSMVSAGVPNSSEETATIENGP

SEQ ID NO: 14 - DNA, 150 bp, HERV-K
gagatagggaaaaaccgccttagggctggaggtgggacctgcgggcagcaatactgctttttaaagcattgagatgtttatgtgtatgcatatctaaaagcacagcacttaatcctttaccttgtctatgatgcaaagatctttgttcac

SEQ ID NO: 15 - DNA, 31 bp, Artificial Sequence
  Primer
catctggtgcccaacgtggaggcttttctct

SEQ ID NO: 16 - DNA, 31 bp, Artificial Sequence
  Primer
aaccgccatcgtcatcatggcccgttctcga

SEQ ID NO: 17 - DNA, 30 bp, Artificial Sequence
  Primer
acagaatctcaaggcagaagaatttttctt

SEQ ID NO: 18 - DNA, 303 bp, HERV-K
catctggtgcccaacgtggaggcttttctctagggtgaaggtacgctcgagcgtggtcattgaggacaagtcgacgagagaatcccgagtacatctacagtcagccttacgtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtactaagaaaaattctt

SEQ ID NO: 19 - DNA, 414 bp, HERV-K
catctggtgcccaacgtggaggcttttctctagggtgaaggtacgctcgagcgtggtcattgaggacaagttgacgagagatcccgagtacatctacagtcagccttgcggagaaaatcagcttcctgtttggatacccactagacatttgaagttctacaatgaacccatcggagatgcaaagaaaagggcctccacagagatgtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttctgtactaagaaaaattcttctgccttgagattctgt

SEQ ID NO: 20 - DNA, 373 bp, HERV-K
tgcccaacgtggaggcttttctctagggtgaaggtacgctcgagcgtggtcattgaggacaagttgacgagagatcccgagtacatctacagtcagccttgcgacatttgaagttctacaatgaacccatcggagatgcaaagaaaagggcctccacagagatgtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttctgtactaagaaaaattcttctgccttgagattctgt

SEQ ID NO: 21 - DNA, 426 bp, HERV-K
catctggtgcccaacgtggaggcttttctctagggtgaaggtacgctcgagcgtggtcattgaggacaagttgacgagagatcccgagtacatctacagtcagccttgcgtatctacagtttaaaacctggtggattgatggagtacaagaacagacatttgaagttctacaatgaacccatcggagatgcaaagaaaagggcctccacagagatgtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttctgtactaagaaaaatttcttctgccttgagattctgt

SEQ ID NO: 22 - DNA, 540 bp, HERV-K
catctggtgcccaacgtggaggcttttctctagggtgaaggtacgctcgagcgtggtcattgaggacaagtcgacgagagatcccgagtacatctacagtcagccttacgacatttgaagttctacaatgaacccatcagagatgcaaagaaaagcwcctccgcggagacggagacatcgcaatcgagcaccgttgactcacaagatgaacaaaatggtgacgtcagaagaacagatgaagttgccatccaccaagaaggcagagccgccaacttgggcacaactaaagaagctgacgcagttagctacaaaatatctagagaacacaaagtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtactaagaaaaattcttctgccttgagattctgt

SEQ ID NO: 23 - DNA, 597 bp, HERV-K
catctggtgcccaacgtggaggcttttctctagggtgaaggtacgctcgagcgtggtcattgaggacaagtcgacgagagatcccgagtacatctacagtcagccttacgacatttgaagttctacaatgaacccatcagagatgcaaagaaaagcacctccgcggagacggagacatcgcaatcgagcaccgttgactcacaagatgaacaaaatggtgacgtcagaagaacagatgaagttgccatccaccaagaaggcagagccgccaacttgggcacaactaaagaagctgacgcagttagctacaaaatatctagagaacacaaaggtgacacaaaccccagagagtatgctgcttgcagccttgatgattgtatcaatggtgtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtactaagaaaaattcttctgccttgagattctgt

SEQ ID NO: 24 - DNA, 581 bp, HERV-K
tcatctggtgcccaacgtggaggcttttctctagggtgaaggtacgctcgagcgtggtcattgaggacaagttgacgagagatcccgagtacatctacagtcagccttgcgacatttgaagttctacaatgaacccatcggagatgcaaagaaaagggcctccacagagatggtaaccccagtcacatggatggataatcctatagaagtatatgttaatgatagtgtatgggtacctggccccacagatgatcgctgccctgccaaacctgaggaagaagggatgatgataaatatttccattgtgtatcgttatcctcctatttgcctagggagagcaccaggatgtttaatgcctgcagtccaaaattgtctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttctgtactaagaaaaattcttctgccttgagattctgt

SEQ ID NO: 25 - DNA, 514 bp, HERV-K
catctggtgcccaacgtggaggcttttctctagggtgaaggtacgctcgagcgtggtcattgaggacaagtcgacgagagatcccgagtacgtctacagtcagccttacgacatttgaagttctacaatgaacccatcggagatgcaaagaaaagggcctccacggagatggtaacaccagtcacatggatggataatcctatagaagtatatgttaatgatagcgaatgggtacctggccccacagatgatcgctgccctgccaaacctgaggaagaagggatgatgataaatatttccattggtctgcaggtgtacccaacggctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtgctaagaaaaattcttctgccttgagattctgt

SEQ ID NO: 26 - DNA, 364 bp, HERV-K
gggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgttctgtactaagaaaaattgttttgccttgagatgctgt

SEQ ID NO: 27 - DNA, 749 bp, HERV-K
yggagatgcaaagaaaagcacctccgcggagacggagacatcgcaatcgagcaccgttgactcacaagatgaacaaaatggtgacgtcagaagaacagatgaagttgtcatccaccaagaaggcagagccgccaacttgggcacaactaaagaagctgacgcagttagctacaaaatatctagagaacacaaaggtgacacaaaccccagagagtatgctgcttgcagccttgatgattgtatcaatggtggtaagtctccctatgcctgcaggagcagctgcagctaactatacctactgggcctatgtgcctttcccgcccttaattcgggcagtcacatggatggataatcctacagaagtatatgttaatgatagtgtatgggtacctggccccatagatgatcgctgccctgccaaacctgaggaagaagggatgatgataaatatttccattgggtatcattatcctcctatttgcctagggagagcaccaggatgtttaatgcctgcagtccaaaattggttggtagaagtacctactgtcagtcccatctgtagattcacttatcacatgtctgcaggtgtacccaacagctccgaagagacagcgaccatcgagaacgggccatgatgacgatggcggttttgtcgaaaagaaaagggggaaatgtggggaaaagcmagaragatcagattgktactgkgtctgtgtagaaagaagtagacataggagactccwttttgc

SEQ ID NO: 28 - protein, 74 aa, HERV-K; identical to SEQ ID NO: 11

SEQ ID NO: 29 - protein, 74 aa, HERV-K; identical to SEQ ID NO: 11

SEQ ID NO: 30 - protein, 44 aa, HERV-K [sequence omitted]

SEQ ID NO: 31 - protein, 74 aa, HERV-K; identical to SEQ ID NO: 11

SEQ ID NO: 32 - protein, 86 aa, HERV-K; identical to SEQ ID NO: 10

SEQ ID NO: 33 - protein, 105 aa, HERV-K
MNPSEMQRKAPPRRRRHRNRAPLTHKMNKMVTSEEQMKLPSTKKAEPPTWAQLKKLTQLATKYLENTKVTQTPESMLLAALMIVSMVSAGVPNSSEETATIENGP

SEQ ID NO: 34 - protein, 127 aa, HERV-K
MVTPVTWMDNPIEVYVNDSVWVPGPTDDRCPAKPEEEGMMINISIVYRYPPICLGRAPGCLMPAVQNCLQVYPTAPKRQRPSRTGHDDDGGFVEKKRGKCGEKQERSDCYCVCVERSRHRRLHFVLY

SEQ ID NO: 35 - protein, 105 aa, HERV-K
MVTPVTWMDNPIEVYVNDSEWVPGPTDDRCPAKPEEEGMMINISIGLQVYPTAPKRQRPSRTGHDDDGGFVEKKRGKCGEKQERSDCYCVCVERSRHRRLHFVMC

SEQ ID NO: 36 - protein, 79 aa, HERV-K [sequence omitted]

SEQ ID NO: 37 - protein, 214 aa, HERV-K
MNSLEMQRKAPPRRRRHRNRAPLTHKMNKMVTSEEQMKLSSTKKAEPPTWAQLKKLTQLATKYLENTKVTQTPESMLLAALMIVSMVVSLPMPAGAAAANYTYWAYVPFPPLIRAVTWMDNPTEVYVNDSVWVPGPIDDRCPAKPEEEGMMINISIGYHYPPICLGRAPGCLMPAVQNWLVEVPTVSPICRFTYHMSAGVPNSSEETATIENGP

SEQ ID NO: 38 - DNA, 418 bp, HERV-K
acatttgaagttctacaatgaacccatcrgagatgcaaagaaaagcacctccgcggagacggagacatcgcaatcgagcaccgttgactcacaagatgaacaaaatggtgacgtcagaagaacagatgaagttgccatccaccaagaaggcagagccgccaacttgggcacaactaaagaagctgacgcagttagctacaaaatatctagagaacacaaaggtgactctgcaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtactaagaaaaattctt

SEQ ID NO: 39 - protein, 129 aa, HERV-K
MNPSEMQRKAPPRRRRHRNRAPLTHKMNKMVTSEEQMKLPSTKKAEPPTWAQLKKLTQLATKYLENTKVTLQVYPTAPKRQRPSRTGHDDDGGFVEKKRGKCGEKQERSDCYCVCVERSRHRRLHFVMY

SEQ ID NO: 40 - DNA, 406 bp, HERV-K
acatttgaagttctacaatgaacccatcrgagatgcaaagaaaagcacctccgcggagacggagacatcgcaatcgagcaccgttgactcacaagatgaacaaaatggtgacgtcagaagaacagatgaagttgccatccaccaagaaggcagagccgccaacttgggcacaactaaagaagctgacgcagttagctacaaaatatctagagaacacaaaggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtactaagaaaaattctt

SEQ ID NO: 41 - protein, 125 aa, HERV-K
MNPSEMQRKAPPRRRRHRNRAPLTHKMNKMVTSEEQMKLPSTKKAEPPTWAQLKKLTQLATKYLENTKVYPTAPKRQRPSRTGHDDDGGFVEKKRGKCGEKQERSDCYCVCVERSRHRRLHFVMY

SEQ ID NO: 42 - DNA, 463 bp, HERV-K
acatttgaagttctacaatgaacccatcagagatgcaaagaaaagcacctccgcggagacggagacatcgcaatcgagcaccgttgactcacaagatgaacaaaatggtgacgtcagaagaacagatgaagttgccatccaccaagaaggcagagccgccaacttgggcacaactaaagaagctgacgcagttagctacaaaatatctagagaacacaaaggtgacacaaaccccagagagtatgctgcttgcagccttgatgattgtatcaatggtggtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtactaagaaaaattctt

SEQ ID NO: 43 - protein, 145 aa, HERV-K
  VARIANT 64: Xaa = Any Amino Acid
MNPSEMQRKAPPRRRRHRNRAPLTHKMNKMVTSEEQMKLPSTKKAEPPTWAQLKKLTQLATKYXLENTKVTQTPESMLLAALMIVSMVVYPTAPKRQRPSRTGHDDDGGFVEKKRGKCGEKQERSDCYCVCVERSRHRRLHFVMY

SEQ ID NO: 44 - DNA, 968 bp, HERV-K
tgtggggaaaagcaagagagatcagattgttactgtgtctgtgtagaaagaagtagacataggagactccattttgttatgtactaagaaaaattcttctgccttgagattctgttaatctatgaccttacccccaaccccgtgctctctgaaacatgtgctgtgtccactcagggttaaatggattaagggcggtgcaggatgtgctttgttaaacagatgcttgaaggcagcatgctccttaagagtcatcaccactccctaatctcaagtacccagggacacaaaaactgcggaaggccgcagggacctctgcctaggaaagccaggtattgtccaacgtttctccccatgtgatagcctgaaatatggcctcgtgggaagggaaagacctgaccgtcccccagcccgacacccgtaaagggtctgtgctgaggaggattagtaaaagaggaaggaatgcctcttgcagttgagacaagaggaaggcatctgtctcctgcctgtccctgggcaatggaatgtctcggtataaaacccgattgtatgctccatctactgagatagggaaaaaccgccttagggctggaggtgggacctgcgggcagcaatactgctttgtaaagcactgagatgtttatgtgtatgcatatctaaaagcacagcacttaatcctttacattgtctatgatgcaaagacctttgttcacatgtttgtctgctgaccctctccccacaattgtcttgtgaccctgacacatccccctcttcgagaaacacccacagatgatcagtaaatactaagggaactcagaggctggcgggatcctccatatgctgaacgctggttccccgggtccccttctttctttctctatactttgtctctgtgtctttttcttttccaaatctctcgtcccaccttacgagaaacacccacaggtgtgtaggggcaacccacccctaca

SEQ ID NO: 45 - DNA, 962 bp, HERV-K
tgtggggaaaagcaagagagatcagattgtcactgtatctgtgtagaaagaagtagacatgggagactccattttgttatgtactaagaaaaattcttctgccttgagattctgtgaccttacccccaaccccgtgctctctgaaacatgtgctgtgtcaaactcagggttaaatggattaagggcggtgcaggatgtgctttgttaaacagatgcttgaaggcagcatgctccttaagagtcatcaccactccctaatctcaagtacccagggacacaaacactgcggaaggccgcagggacctctgcctaggaaagccaggtattgtccaaggtttctccccatgtgatagtctgaaatatggcctcgtgggaagggaaagacctgaccgtcccccagcccgacacccgtaaagggtctgtgctgaggaggattagtaaaagaggaaggcatgcctcttgcagttgagacaagaggaaggcatctgtctcctgcccgtccctgggcaatggaatgtctcggtataaaaccggattgtacgttccatctactgagatagggaaaaaccgccttagggctggaggtgggacctgcgggcagcaatactgctttttaaagcattgagatgtttatgtgtatgcatatctaaaagcacagcacttaatcctttaccttgtctatgatgcaaagatctttgttcacgtgtttgtctgctgaccctctccccactattgtcttgtgaccctgacacatccccctctcggagaaacacccacgaatgaccaataaatactaaagggaactcagaggctggcgggatcctccatatgctgaacgctggttccccgggcccccttatttctttctctacactttgtctctgtgtctttttctttcctaagtctctcgttccaccttacgagaaacacccacaggtgtggaggggcaacccacccctaca

SEQ ID NO: 46 - DNA, 968 bp, HERV-K
tgtggggaaaagcaagagagatcagattgttactgtgtctgtgtagaaagaagtagacatgggagactccattttgttatgtgctaagaaaaattcttctgccttgagattctgttaatctatgaccttacccccaaccccgtgctctctgaaacatgtgctgtgtcaactcagggttgaatggattaagggcggtgcaggatgtgctttgttaaacagatgcttgaaggcagcatgctccttaagagtcatcaccactccctaatctcaagtacccagggacacaaaaactgcggaaggccgcagggacctctgcctaggaaagccaggtattgtccaaggtttctccccatgtgatagtctgaaatatggcctcgtgggaagggaaagacctgaccatcccccagcccgacacccataaagggtctgtgctgaggaggattagtataagaggaaggcatgcctcttgcagttgagacaagaggaaggcatctgtctcctgcctgtccctgggcaatggaatgtctcggtataaaacccgattgtatgctccatctactgagatagggaaaaaccgccttagggctggaggtgggacctgcgggcagcaatactgccttgtaaagcattgagatgtttatgtgtatgcatatctaaaagcacagcacttaatcctttacattgtctatgatgcaaagacctttgttcacgtgtttgtctgctgaccctctccccacaattgtcttgtgaccctgacacatccccctctttgagaaacacccacagatgatcaataaatactaagggaactcagaggctggcgggatcctccatatgctgaacgctggttccccggttccccttatttctttctctatactttgtctctgtgtctttttcttttccaaatctctcgtcccaccttacgagaaacacccacaggtgtgtaggggcaacccacccctaca

SEQ ID NO: 47 - DNA, 968 bp, HERV-K
tgtggggaaaagcaagagagatcagattgttacagtgtctgtgtagaaagaagtagacataggagactccattttgttctgtactaagaaaaattcttctgccttgaaattctgttaatctataaccttacccccaaccccgtgctctttgaaacatgtgctgtgtcaactcagagttaaatggattaagtgcggtgcaagatgtgctttgttaaacagatgcttgaaggcagcatgctccttgagagtcatcaccactccctaatctcaagtacccagggacacaaaaactgcggaaggcctcagggacctctgcctaggaaagccaggtattgtccaaggtttctccccatgtgatagtctgaaatatggcctcgtgggaagggaaagacctgaccatcccccagcccgacacccgtaaagggtctgtgctgaggaggattagtaaaagaggaaggaacgcctcttgcagttgagacaagaggaaggcatctgtctcctgcctgtccctgggcaatggaatgtcccggtataaaacccgattgtatgctccatctactgagatagggaaaaaccgccttagggctggaggtgggacctgcgggcagcaatactgctttgtaaagcattgagctgtttatgtgtatgcatatctaaaagcacagcacttaatcctttacattgtctatgatgcaaagacctttgttcacgtgtttgtctgctgaccctctccccacaattgtcttgtgaccctgacacatccccctcttcgagaaacacccacgaatgatgaataaatactaagggaactcagaggctggcgggatcctccatatgctgaacgctggttccccgggtccccttacttctttctctgtactttgtctctgtgtctttttctttcctaagtctctcgttccaccttacgagaaatacccacaggtgtggaggggcaacccacccctaca

SEQ ID NO: 48 - DNA, 968 bp, HERV-K
tgtggggaaaagcaagagagatcagattgttactgtgtctgtgtagaaagaagtagacataggagactccattttgttctgtactaagaaaaattcttctgccttgagattctgttaatctataaccttacccccaaccccgtgctctctgaaacatgtgctatgtcaactcagagttgaatggattaagggcggtgcaagatgtgctttgttaaacagatgcttgaaggcagcacgctccttaagagtcatcaccactccctaatctcaagtacccagggacacaaaaactgcggaaggccgcagggacctctgcctaggaaagccaggtattgtccaaggtttctccccatgtgatagtctgaaatatggcctcgtgggaagggaaagacctgaccatcccccagcccgacacctgtaaagggtctgtgctgaggaggattagtataagaggaaggcatgcctcttgcagttgagacaagaggaaggcatctgtctcctgcccgtccctgggcaatggaatgtctcggtataaaacccgattgtatgttccatctactgagatagggaaaaaccgccttagggctggaggtgggacctgcgggcagcaatactgctttgtaaagcattgagatgtttatgtgtatgcatatctaaaagcacagcacttaatcctttaccttgtctatgatgcaaagacctttgttcacgtgtttgtctgctgaccctctccccacgattgtcttgtgaccctgacacatccccgtcttcgagaaacacccacgaatgatcaataaatactaagggaactcagaggctggcgggatcctccatatgctgaacgctggttccccaggtccccttatttctttctctatactttgtctctgtgtctttttcttttccaagtctctcgttccatcttacgagaaacacccacaggtgtggaggggcaacccacccctaca

SEQ ID NO: 49 - DNA, 150 bp, HERV-K
gagatagggaaaaaccgccttagggctggaggtgggacctgcgggcagcaatactgctttgtaaagcactgagatgtttatgtgtatgcatatctaaaagcacagcacttaatcctttacattgtctatgatgcaaagacctttgttcac

SEQ ID NO: 50 - DNA, 258 bp, HERV-K
atgtttgtctgctgaccctctccccacaattgtcttgtgaccctgacacatccccctcttcgagaaacacccacagatgatcagtaaatactaagggaactcagaggctggcgggatcctccatatgctgaacgctggttccccgggtccccttctttctttctctatactttgtctctgtgtctttttcttttccaaatctctcgtcccaccttacgagaaacacccacaggtgtgtaggggcaacccacccctaca

SEQ ID NO: 51 - DNA, 174 bp, HERV-K
gtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtactaa

SEQ ID NO: 52 - DNA, 50 bp, Artificial Sequence
  Primer
gtgtacccaacagctccgaagagacagcgaccatcgagaacgggccatga

SEQ ID NO: 53 - DNA, 26 bp, Artificial Sequence
  Primer
agagaaaagcctccacgttgggcacc

SEQ ID NO: 54 - DNA, 18 bp, Artificial Sequence
  Primer
gtaggggtgggttgcccc

SEQ ID NO: 55 - DNA, 27 bp, Artificial Sequence
  Primer
aaaccgccttagggctggaggtgggac

SEQ ID NO: 56 - DNA, 18 bp, Artificial Sequence
  Primer
tgcgggcagcaatactgc

SEQ ID NO: 57 - DNA, 28 bp, Artificial Sequence
  Primer
taaagcactgagatgtttatgtgtatgc

SEQ ID NO: 58 - DNA, 24 bp, Artificial Sequence
  Primer
gcacagcacttaatcctttacatt

SEQ ID NO: 59 - DNA, 22 bp, Artificial Sequence
  Primer
gtttgtctgctgaccctctccc

SEQ ID NO: 60 - protein, 14 aa, Artificial Sequence
  V5 tag
GKPIPNPLLGLDST

SEQ ID NO: 61 - protein, 5 aa, HERV-K
LQVYP

SEQ ID NO: 62 - protein, 5 aa, HERV-K
APKRQ

SEQ ID NO: 63 - protein, 6 aa, HERV-K
DDGGFV

SEQ ID NO: 64 - protein, 4 aa, HERV-K
KKRG

SEQ ID NO: 65 - protein, 6 aa, HERV-K
LHFVLY

SEQ ID NO: 66 - protein, 56 aa, HERV-K
YPTAPKRQRPSRTGHDDDGGFVEKKRGKCGEKQERSDCYCVCVERSRHRRLHFVLY

SEQ ID NO: 67 - protein, 59 aa, HERV-K
LQVYPTAPKRQRPSRTGHDDDGGFVEKKRGKCGEKQERSDCYCVCVERSRHRRLHFVLY

SEQ ID NO: 68 - protein, 58 aa, HERV-K
LQVYPAAPKRQQPARMGHSDDGGFVKKKRGGYVRKREIRLSLCLCRKGRHKKLHFVLY

SEQ ID NO: 69 - protein, 74 aa, HERV-K; identical to SEQ ID NO: 11

SEQ ID NO: 70 - protein, 74 aa, HERV-K
MNPSEMQRKGPPQRCLQVYPTAPKRQRPSRTGHDDDGGFVEKKRGKCGEKQERSDCYCVCVERSRHRRLHFVMY

SEQ ID NO: 71 - protein, 74 aa, HERV-K
MNPSEMQRKGPPQRCLQVYPTAPKRQRPSRTGHDDDGGFVEKKRGKCGEKQERSDCYCVCVERSRHRRLHFVLC

SEQ ID NO: 72 - protein, 74 aa, HERV-K
MNPSEMQRKGPPQRCLQVYPTAPKRQRPSRTGHDDDGGFVEKKRGKCGEKQERSDCYCVCVERSRHRRLHFVMC

SEQ ID NO: 73 - protein, 74 aa, HERV-K
MNPSEMQRKGPPRRCLQVYPTAPKRQRPSRTGHDDDGGFVEKKRGKCGEKQERSDCYCVCVERSRHRRLHFVLY

SEQ ID NO: 74 - protein, 74 aa, HERV-K
MNPSEMQRKGPPRRCLQVYPTAPKRQRPSRTGHDDDGGFVEKKRGKCGEKQERSDCYCVCVERSRHRRLHFVMY

SEQ ID NO: 75 - protein, 74 aa, HERV-K
MNPSEMQRKGPPRRCLQVYPTAPKRQRPSRTGHDDDGGFVEKKRGKCGEKQERSDCYCVCVERSRHRRLHFVLC

SEQ ID NO: 76 - protein, 74 aa, HERV-K
MNPSEMQRKGPPRRCLQVYPTAPKRQRPSRTGHDDDGGFVEKKRGKCGEKQERSDCYCVCVERSRHRRLHFVMC

SEQ ID NO: 77 - DNA, 1010 bp, HERV-K
tgtagggaaaagaaagagagatcacactgttactgtgtctatgtagaaaaaggaagacataagaaactccattttgatctgtactaagaaaaattcttctgctttgaaatgctattaatctgtaaccctagccccaaccctgtgctcacagaaacatgcgctgtattgactcaaggttaatggatttagggctgtgcaggatgtgctttgttaacaatgtgtttgaaggcagtatgcttggtaaaggtcatcgccattctccagtcttgagtacccagggacacaatgcactgtggaaagccatggggacctctgcccaagaaagcctgggtgttgtccaggcttccccacactgagacagcctgagatgtggcctcgttggaagggaaagaccttacattatagtcccccagccggacacccataaaaggtctgtgctgaggaggattactgaaagaggaaggcctctttgcagttaagaggaaagcatctgtctcatgatcccctgggaatggaatgtcttggtgtaaaacctgatcgtacattctatttactgagataggagaaaaccgccctatggctggaggtgagacatgctggtggcaataccgatctttactgcacggcaatactgatctttactgcactgagatgtttatgtaaagttaaacataaatctagcctacgtgcacattcaggcatagcacctttccttaaacttatttatgacacagagtcttttgttcacgtgttttcctgttgaccctctctccaccattaccctatagtcctgccacatccccctcactgagatagtagagataatgatcaataaatactgagggaattcagaaaccagtgccggtgcaggtcctcacttgctgagtgccggtcccctgggcccacttttcttcctctatgctttacctctgtgtcttatttcttttctcagtctctcgtctccaccttgcgagaaatacccacaggtgtggaggggctggcccccttca

SEQ ID NO: 78 - protein, 57 aa, HERV-K
VYPTAPKRQQPSRTGHDNDGSFVEKRRGKCGEKKERSDCYCVCVERSRHRRLHFVLY

SEQ ID NO: 79 - protein, 57 aa, HERV-K
VFPTALKRQRPSRMGHDDDGGFVEKKRGKCGEKKERSDCYCVCVERSRHRRLHFVLY

SEQ ID NO: 80 - protein, 57 aa, HERV-K
VYPTAPKRQQPSRTGHDDDGGFVKKKRGKCGEKKERSDCYCVCVERSRHRILHFVLY

SEQ ID NO: 81 - protein, 65 aa, HERV-K
LQVYPAAPKRERPVRTGHDDDGGFLKKKRGICREKKERSDGYCVYVEKEDIRNFILICTLNNCFA

SEQ ID NO: 82 - protein, 42 aa, HERV-K
VYPAAPKRQRPSRTSHDDDGGLSKRKWGNVGKREIRLLLCLC

SEQ ID NO: 83 - protein, 56 aa, HERV-K
VYPTAPKRQRPSRTGHDDDGGFVEKKRGKCGEKQERSDCYCVCVERSRHGRLHFVM

SEQ ID NO: 84 - protein, 57 aa, HERV-K
VYLTAPKRQRPSRTGHDDDGGFVKKKRGKCGEKKERSDCYCVCVERSRHRILHFVLY

SEQ ID NO: 85 - protein, 46 aa, HERV-K
VYPTALKRQRPKRMGHDDYGSSVKKKRGICRGKKERSDCYCVYVEK

SEQ ID NO: 86 - protein, 57 aa, HERV-K
VYPTAPKRQRPLRTGHDDDGGFVKKKRGKCGEKKERSDCYCVCVERSRHRRLHFVLY

SEQ ID NO: 87 - protein, 57 aa, HERV-K
VYPTAPKRQQPSRTGHDDDGCFLEKKRGKCGEKKERSDCYCVCVERSRHRRLHFVLY

SEQ ID NO: 88 - protein, 57 aa, HERV-K
VYRTALKRQRPSRMGHDDDGSFVEKKRGKCGEKKERSDCYCVCVERSRHRRLHFVLY

SEQ ID NO: 89 - protein, 57 aa, HERV-K
VYPTAPKRQRPSRTGHDDDGGFVEEKRGKCGAKKERSDCYCVCVERSRHRRLHFVLY

SEQ ID NO: 90 - protein, 54 aa, HERV-K
VYPTAPKRQQPSRNSHDDDGGFVEKGEMWGKERSDCYCVCVERSRHRRLHFVLY

SEQ ID NO: 91 - protein, 45 aa, HERV-K
VYPTAPKRQRPSRTGHDDDGGFVEKKRRKSGEKREIRLLLCLCRK

SEQ ID NO: 92 - protein, 57 aa, HERV-K
VYPTAPKRQRPSRTGHDDDGGFVRKKRGKCGEKKERSDCYCVCVERTRHRRFHFVLY

SEQ ID NO: 93 - protein, 51 aa, HERV-K
VYPTAPKRQRPSRTGQYDDGSFVKKKRGRKEKGEMWGKEREIRLLLCLCRK

SEQ ID NO: 94 - protein, 46 aa, HERV-K
VYPAAPKRQRPVRMGHNDDVSFVKKKRGICREKKERSDCYCVYVEK

SEQ ID NO: 95 - protein, 57 aa, HERV-K
VYPTAPKRQRPSRMGHDDYGGFVEKKRGKCGEKKERSDCYCVCVERSRHRRLHFVLY

SEQ ID NO: 96 - protein, 57 aa, HERV-K
VYPTAPKRQQPSRMGHDDDGGFVKKKRGKCGEKKERSDCYFVCVERSRHRRLHFVLY

SEQ ID NO: 97 - protein, 57 aa, HERV-K
VYPTAPKRQRPSRTGHDDDGGFVEKKRGKCGEKQERSDCYCVCVERSRHRRLHFVMY

SEQ ID NO: 98 - protein, 57 aa, HERV-K
VYPTAPKRQRPSRTGHDDDGGFVEKKRGKCGEKQERSDCHCVCVERSRHGRLHFVMY

SEQ ID NO: 99 - protein, 54 aa, HERV-K
VYPTAPKRQRPSRTGHDDSGGFVEKKRGKCGEKKERSDCYCVCVERSRHRRLHF

SEQ ID NO: 100 - protein, 57 aa, HERV-K
VYPTAPKRQRPSRMGHDDDGGFVEKKRGKCGEKKERSDCYCVCVERSRHRRLHFVLY

SEQ ID NO: 101 - protein, 57 aa, HERV-K
VYPTARKRQQPSRTGHDDDGGFVVKKRGKCGEKKERSDCYCVCVERSRHRRLHFVLY

SEQ ID NO: 102 - protein, 57 aa, HERV-K
VYPTAPKRQRPSRTGHDDDGGFVEKKRGKCGEKKERSDCYCVCVERSRHRRLHFVLY

SEQ ID NO: 103 - protein, 45 aa, HERV-K
VYPTAPKRQRPSRTGHDDDGGFVEKKEKGEMWGKEREIRLLLCLC

SEQ ID NO: 104 - protein, 57 aa, HERV-K
VYPTAPKRQRPSRTGHDDDGGFVEKQRGKCGEKKERSDCYCVCVERSRHRRLHFVLY

SEQ ID NO: 105 - protein, 57 aa, HERV-K
VYPTAPKRQQPSRTGHDEDGGFVERKRGNCGEKKERSDCYCVCVERSRHRRLHFALY

SEQ ID NO: 106 - protein, 57 aa, HERV-K
VYPTAPKRQRPSRTGHDDDGGFVEKKRGKCGGKNERSDCYCVCVERSRHRRLHFVLY

SEQ ID NO: 107 - protein, 57 aa, HERV-K
VYPTAPKRQRPSRMGHDDDGGFVEKKRGKCGEKQERSDCYCVCIERSRHRRLHFVLY

SEQ ID NO: 108 - protein, 65 aa, HERV-K
LQVYPTAPKRQQPARTGHNDDGSFVKKKRGICREKKEISDCYCIFVEKEDIRNSILTCTVNNCFA

SEQ ID NO: 109 - protein, 55 aa, HERV-K
VYPAAPKRQRPSRTSHDDDGSFVKKKRVMWGKERSDCYCVYVERSRHKRLHFVLY

SEQ ID NO: 110 - protein, 83 aa, HERV-K
LQVYPAAPERQRPGRRGHDDGGGFVKTKRGICRGKKERSDCYCVYIEREDIRDSILKKICTLSNCFAEMLLICSFAPATLPQP

SEQ ID NO: 111 - protein, 57 aa, HERV-K
VYPTAPKRQRPSRTGHDDNGGFVEKKRGKCGEKKERSDCYCVCVERSRHRRLHFVLY

SEQ ID NO: 112 - protein, 83 aa, HERV-K
LQVYPAAPERQRPGRRGHDDGGGFVKTKRGICRGKKERSDCYCVYIEREDIRDSILKKNCTLNNCFAEMFLICSFAPATFPQP

SEQ ID NO: 113 - protein, 36 aa, HERV-K
VYPTAPKRQRPSRTGHDDDGGFVEKKRGKCGEKRDQ

SEQ ID NO: 114 - protein, 57 aa, HERV-K
VYPTAPKRQRPSRTGHDDSGSFVKKKRGKCGEKKERSDCYCVCVERSRHRRLRFVLY

SEQ ID NO: 115 - protein, 57 aa, HERV-K
VYPTAPKRQRPSRMGHDDDGGFVEKQRGKCREKKERSDCYCVCVERSWHRRLHFVLY

SEQ ID NO: 116 - protein, 57 aa, HERV-K
VYPTALKRQQPSRTGHDDDGGFVKKKRGKCGEKQERSDCHCVCVERSRHRRLHFVMY

SEQ ID NO: 117 - protein, 57 aa, HERV-K
VYPTARKRQRPSRTGHDDDGGFVEKKRGKCGEKQERSDCYCVCVERSRHRRLHFVLY

SEQ ID NO: 118 - protein, 46 aa, HERV-K
KPRRTKTQHTRISGTHSTCGEKQERSDCYCVCVERSRHRRLHFVLY

SEQ ID NO: 119 - protein, 46 aa, HERV-K
VYPAAPERQRPARRGHDDGGGFVKTKRGICRVKKERSDCYCVYIER

SEQ ID NO: 120 - protein, 83 aa, HERV-K
LQVYPAAPERQRPARRGHDDGGGFVKTKMGICREKKERSDCYCVYIEREDIRDSILKKTCTLNNCFAEMLLICSFAPATLPQP

SEQ ID NO: 121 - protein, 57 aa, HERV-K
VYPTAPKRQRPSRKGHDDDGGFVEKKRGKCGEKKERSDCYCVCVERSRHRRHHFVLY

SEQ ID NO: 122 - protein, 75 aa, HERV-K
LQVYTTAPERQRPARTGHDDDGGFVKKKRGKCREKKERSDCHCAYVEREDIRDSILKKTCTLNNCFAEMLLICSF

SEQ ID NO: 123 - protein, 57 aa, HERV-K
VYPTAPKRQRPSRTGHDDDGGFVEKKREKCGEKKERSNCYCVCVERSRHRRLHFVLY

SEQ ID NO: 124 - protein, 58 aa, HERV-K
LQVYPTALKRQQPSRTGHDDDGSFVEKKRGKCGEKKERSDCYCVCVERSRHRRFQKKK

SEQ ID NO: 125 - protein, 55 aa, HERV-K
VYPTALKRQRPSRTGHDDDGGFVEKKREKCGEKKDQIVTVSVERSRHRRLHFVLY

SEQ ID NO: 126 - protein, 57 aa, HERV-K
VYPTAPKRQPPSRTGHDDDGGFVLKKRGKCGEKKERSDCYHVCVERSRHRRHHFVLY

SEQ ID NO: 127 - protein, 56 aa, HERV-K
VYPTAPKRQRPSRTGHDDDGGFVEKKRGKCGEKKERSDCYCVCLERSRHRRLHFVL

SEQ ID NO: 128 - protein, 57 aa, HERV-K
VYSTAPKRQRPSRTGHDDDGGFVEKKRGKCGEKKERSDCYCVCVERSRHSRLHFVLY

SEQ ID NO: 129 - protein, 57 aa, HERV-K
VYPTAPKRQRPSRTGHDDNGSFVEKKKGKCGEKKERSDCYCVCVERSRHRRLHFVLY

SEQ ID NO: 130 - protein, 57 aa, HERV-K
VYPTAPKRQQPSRTGHDDDGGFVKKKRGKCGEKKERSDCYCVCVERSRHRRLHFVLY

SEQ ID NO: 131 - protein, 57 aa, HERV-K
VYPTASKRQPPSGTDHDDDGGFVKKKRGKCGEKKERSDCYCVCVERSRHRRLHFLLY

SEQ ID NO: 132 - protein, 52 aa, HERV-K
PSEQRPRETNGCHSGPDPRHSQEGPCGEKKEISDCYCVYVERSRHKRLHFVV

SEQ ID NO: 133 - protein, 57 aa, HERV-K
VYPTAPKRQRPSRTGSDDDGGFVEKKRGKCGEKKERTDCYCVCVERSRHRRLHFVLY

SEQ ID NO: 134 - protein, 36 aa, HERV-K
VYPTAPKKQQPSIMGHDDDGGFVKKKRGKCGEKRDQ

SEQ ID NO: 135 - protein, 57 aa, HERV-K
VYPTAPKRQRPSRTGHDDDGGFVKKKRGKCGEKKEKSDCYCVCVERSRHRRLHFVLY

SEQ ID NO: 136 - protein, 63 aa, HERV-K
SPSAQRPPRLGGVPNSSLRTGHDDDGGFVEWRGGKCGEKIDKSDCCCVCVEGSRRRRLHFVLY

SEQ ID NO: 137 - protein, 57 aa, HERV-K
VYPTAPKRQRPSRTGHDDDGGFVKKKRGKCGEKKERSDCYCVCVERSRHRRLHFVLY

SEQ ID NO: 138 - protein, 57 aa, HERV-K
VYPTAPKRQRPSRTGHDDDGGFVEKKMGKFGEKKERSDCYCVCVERSRHRRLHFVLY

SEQ ID NO: 139 - protein, 57 aa, HERV-K
VYPTAPKRQRPLRMGHDDDGSFVKKKRGKCGEKKERSDCYCVCVERSRHRRLHFVLY

SEQ ID NO: 140 - protein, 36 aa, HERV-K
VYPTAPKRQRLSRTGHDDDGGFVEKKRGKCGEKKDQ

SEQ ID NO: 141 - protein, 57 aa, HERV-K
VYPTAPKRQQPSRTGHDDDGGFVKNKRGKRGEKKERSDCYCVCVERSRHRRHPFVLY

SEQ ID NO: 142 - protein, 57 aa, HERV-K
VYPTALKRQRPSRTGHDNDGGFVEKKRGKCGEKQERSDCHCVCVERSRHRRLHFVMY

SEQ ID NO: 143 - protein, 57 aa, HERV-K
VHPTAPKRQRPSRTGHDDNGSFVEKKRGKCGEKKERSDCYCVCVERSRHRRLHFVLY

SEQ ID NO: 144 - protein, 85 aa, HERV-K
EVYPIAPKRQRPSRTGHDDDGGFVEKKRGKCGEKKERSDCYCVCVERSRYRRLHFVLYLEKFFCLGMLLIYNLTPNPVLSETCAV

SEQ ID NO: 145 - protein, 57 aa, HERV-K
VYPTAPKRQRPLRMGHDDDGGFVKKKRGKCGEKKERSDCYCVCVERSRHRRLHFVLY

SEQ ID NO: 146 - protein, 57 aa, HERV-K
VYPTAPKRQRPSRTGHDDDGGFVEKKRGKCGGKKERSDCSCVCVERSRHRRLHFVLY

SEQ ID NO: 147 - protein, 57 aa, HERV-K
VYPTALKRQRPSRTGHDDDGGFVEKKRRKCGEKKERTDCYCVCVERSRHRGLHFVLY

SEQ ID NO: 148 - protein, 57 aa, HERV-K
VYPTAPKRQRPSRTGHDDDGGFVEKKRRKCGEKKERTDCYCVCVERSRHRGLHFVLY

SEQ ID NO: 149 - protein, 83 aa, HERV-K
EVYPTSPKRQQPSRMGHDDDGGFVAKKRGKCGEKKERSDCYCVCVERSRHRRLHFVLYLEKFFCLGMLLIYNFTPNHVLSETC

SEQ ID NO: 150 - protein, 57 aa, HERV-K
VYPTASKRQPPSGTDHDDDGSFVKKKRGKCGEKEERSDCYCVCVERSRHRRLHFLLY

SEQ ID NO: 151 - protein, 57 aa, HERV-K
VYPTAPKRQRPSRTGHDDDGGFVEKKRGKCGEKKERSDCYCVCVERSRHRRHHFVLY

SEQ ID NO: 152 - protein, 57 aa, HERV-K
VYPTAPKRQRPLRMGHDDDGGFVEKKMGKCGEKKERSDCYCVCVERSRHRRLHFVLY

SEQ ID NO: 153 - protein, 57 aa, HERV-K
VYPTAPKRQRPWRMGHDDDGGFVEKKRGKCGEKKERSDCHCVCVERSRHRRLHFVLY

SEQ ID NO: 154 - protein, 57 aa, HERV-K
VYPTAPKRQRPWRMGHDDDGGFVEKKRGKCGEKKERSDCYCVCVERSRHRRLHFVLY

SEQ ID NO: 155 - protein, 56 aa, HERV-K
YPTALKRQRPSRTGHDDYGSFVKKKRGKCGEKKERSDCYCVCVERSRHSRLHFVLY

SEQ ID NO: 156 - protein, 62 aa, HERV-K
APTRQPPCLRGVPNSSLRTGHDDDGGFVEQKRGKCREKKERSDCYCVCVERSRHRRLHFVLY

SEQ ID NO: 157 - protein, 57 aa, HERV-K
VYPTAPKRQRPSRTGHDDDGGFVKKKRGKCGEKKERSDCYCVCVERSIHRRLHFVLY

SEQ ID NO: 158 - protein, 57 aa, HERV-K
VYPTAPKRQRPSRTGHDDDGGFVEKKRGKCGEKQERSDCYCVCVERSRHGRLHFVLY

SEQ ID NO: 159 - protein, 57 aa, HERV-K
VYPTAPKRQQPSRMGHDDNGGFVEKKRGKCGEKKERSDYYCVCVERSRHRRLHFVLY

SEQ ID NO: 160 - protein, 57 aa, HERV-K
VYPTAPKRQRPSRTGHDDDGGFVEKKRGKCREKKERSDCYCVCVERSRHRRLHFVLY

SEQ ID NO: 161 - protein, 55 aa, HERV-K
PSGRCAQQLIEKGHDDNGGLVEWRRGKCGEKRERSDCCCVCVEGGRRGRLHFVLY

SEQ ID NO: 162 - protein, 56 aa, HERV-K
YPTAPKRQRPSRKSHDDDGGFVEKKRGKYGEKKERSDCYCVCVERSRHRRLHFVLY

SEQ ID NO: 163 - protein, 35 aa, HERV-K
VYPTAPKRQRPSRTGHDDDGGFVQKKRGKWEKRDQ

SEQ ID NO: 164 - protein, 68 aa, HERV-K
LQVYPAAPKRQRPLRMGDDDDGGFVKKKRGKCGEKKERSDCYCVYVEKEDIRNSILICIKKNCSALRC

SEQ ID NO: 165 - protein, 37 aa, HERV-K
RRERPSRTSHDDNGGFVEKKGEMWGKERDIRLLLCLC

SEQ ID NO: 166 - protein, 57 aa, HERV-K
VYPTAPKRQRPSRTGHDDDGSFVKNKRGKCGEKKERSDCCCVCVERSRHRRLHFVLY

SEQ ID NO: 167 - protein, 57 aa, HERV-K
VYPAAPKRQRPLRTGHDDDGGFVEKKRGKCGEKKQKSDCYCVCVERDRHRRLHFVLY

SEQ ID NO: 168 - protein, 57 aa, HERV-K
VYPTAPKRQRPSRTGHDDNGSFVEKKRGKCGEKKERSDCYCVCVERSRHRRLHFVLY

SEQ ID NO: 169 - protein, 44 aa, HERV-K
VYPTAPKRQQPSRTGHDDDGSFVKKKRGKCGEKRDQMLLCLCRK

SEQ ID NO: 170 - protein, 57 aa, HERV-K
VYPTAPKRQRPLRTGHDDDGGFVEKKRGKCGEKKERSDCYCVCVERSRHKRLHFVLY

SEQ ID NO: 171 - protein, 46 aa, HERV-K
VYAAALERQRPARNGHDDDGGFVKKKRGIYREKKERSDCYCVYVER

SEQ ID NO: 172 - protein, 53 aa, HERV-K
PPEQRPREMNGCHSGPDLRHSQEGPCGEKKEISDCYCVYVERSRRKRLHFVLY

SEQ ID NO: 173 - protein, 43 aa, HERV-K
VYPIAPKRQRTSRTGHDDNGGFVEKKREMWGKEREIRLLLCLC

SEQ ID NO: 174 - protein, 57 aa, HERV-K
VYPTAPKRQQPSRTGHDDDGGFVQKKRGKCGEKKERSDCYCVCVERSRHRRLHFVLH

SEQ ID NO: 175 - protein, 57 aa, HERV-K
VYPTAPKRQRPWRTGHDDDGGFVEKKRGKCGEKKERSDCYCVCVERSRHRRLHFVLY

SEQ ID NO: 176 - protein, 57 aa, HERV-K
VYPTAPKRQRPSRTRHDDDGSFVEKRRGKCGEKQERSDCYCVCVERSRHRRLHLVMY

SEQ ID NO: 177 - protein, 57 aa, HERV-K
VYPTAPKRQRPSRTGQDDDGSFVEKRRGKCGEKQERSDCYCVCVERSRHRRLHFLMY

SEQ ID NO: 178 - protein, 57 aa, HERV-K
VYPTAPKRQRPSRTGHEDDGGFVKKKRGKCGEKKERSDCYCVCVERNRHRRLHFVLY

SEQ ID NO: 179 - protein, 52 aa, HERV-K
PPEQRPREMNGCHSGPDPRHSQEGPCGEKKEISDCYCVYVERSRRKRLHFVV

SEQ ID NO: 180 - protein, 65 aa, HERV-K
VFTTAEQGRTPAPGTQRDFAKGMDLAGPRGCLCREKKERSHCYCVYVEKEDINSILSCTKKNYFA

SEQ ID NO: 181 - protein, 56 aa, HERV-K
VYPAAPKRQQPARMGHSDDGGFVKKKRGGYVRKREIRLSLCLCRKGRHKKLHFDLY

SEQ ID NO: 182 - protein, 52 aa, HERV-K
PPEQRPREMNGCHSGPDPRHSQEGPCGEKKEISDCYCVYVERSRRKRLHFVL

SEQ ID NO: 183 - protein, 55 aa, HERV-K
VYPTAVKRQRPSRTGHDDDGGFVKKKRGKCGEKKERSDCYCVCVERSRHRRLHFL

SEQ ID NO: 184 - protein, 36 aa, HERV-K
VYPTALKRQRPSRTGHDDDGSFVEKKRGKCGEKRDQ

SEQ ID NO: 185 - protein, 55 aa, HERV-K
PTALKRQRPSRTGHDDDGSFVEKKRGKCGEKKERTDCYCVCVERSRHRRLHFVLY

SEQ ID NO: 186 - protein, 46 aa, HERV-K
VYPAAPERQRPARRGHNDGGGFVKKKRGICREKKERSDCYCVYIER

SEQ ID NO: 187 - protein, 36 aa, HERV-K
VYPTAPKRQRPLRTGHDDDGSFVEKKRRKCGEKREQ

SEQ ID NO: 188 - protein, 57 aa, HERV-K
LYPTAPKRQRPSRTGHDDDSGFVEKKRGKCGEKQERSDCYCVCVERSRHRRLHFVMY

SEQ ID NO: 189 - protein, 74 aa, HERV-K
DSDRPERRGHDDGGGFVKTKRGICREKKERSDCYCVYIEREDIRDSILKKTCTLNSCFDRDSCLSAFMCLLLPQ

SEQ ID NO: 190 - protein, 63 aa, HERV-K
SPSAQRPPRLGGVPNSSLRTGHDADGGFVEWKRGKCGEKIERSDCYCVCIERSRHRRLHFVLY

SEQ ID NO: 191 - protein, 34 aa, HERV-K
VYPTSPKRQRPSRTGHDDDGGFVEKKRGNVGKRK

SEQ ID NO: 192 - protein, 57 aa, HERV-K
VYPTAPKRQRPLRMGHGDDGGFVEKKRGKCREKKERSDCYCVCVERSRHRRLHFVLY

SEQ ID NO: 193 - protein, 81 aa, HERV-K
LQVYPAAQERHRPARRGHDDGGGFVKTKRGIYREKKERSDCYCVYTEREDIRDSILKKTCTLNNCFAEMLLICSFAPATLP

SEQ ID NO: 194 - protein, 46 aa, HERV-K
VYPAATEKQRPARTGHDDDGGVVKKKRGKCREKKEGSDCHCVYAER

SEQ ID NO: 195 - protein, 56 aa, HERV-K
YPTAPKRQQPWRTGLDDLGGFFEKKRGNFGEKKGGSDFYSVCVERSRHRGPHFVLY

SEQ ID NO: 196 - protein, 57 aa, HERV-K
VYPTAPKRQQPWRTGHDDHGGFVEKKRGKCGEKKERSDCYYVCVERSRHRRLHFVLY

SEQ ID NO: 197 - protein, 56 aa, HERV-K
VYPTAPKRQRPSRMGHDDDGGFVEKKRGKCGEKKERSDCYCVCVESRHRRLHFVLY

SEQ ID NO: 198 - protein, 45 aa, HERV-K
VYPTAPKRQRPSRTGHDDDGGFVEKRRGKCGEKKEIRLLLCLCRK

SEQ ID NO: 199 - protein, 57 aa, HERV-K
VYPTAPKRQQPSRTGHDDDGSFVEKKRGKCGEKKERSDCYCVCVERSRHRRLHFVLY

SEQ ID NO: 200 - protein, 46 aa, HERV-K
VYPAAPERQRPARMGHDDDGGFVKKKRGKCREKKERSDCHSVYVEK

SEQ ID NO: 201 - protein, 57 aa, HERV-K
VYPTAPKRQRSSRTGRDNDGGFVKKKRGKCGEKKERSDCYCVCVGRSRHRRLHFVLY

SEQ ID NO: 202 - protein, 46 aa, HERV-K
VYPAAPERQQPARRGHDDGGGFVKKKRGICREKKERSDSYCVYIER

SEQ ID NO: 203 - protein, 53 aa, HERV-K
VYPAAPERQRPVRRGHDDDGGFVKKKRGKCREKREIRLSLCLCRKGRHKRLHF

SEQ ID NO: 204 - protein, 57 aa, HERV-K
VYPTAPKRQRPSRTGHDDDGGFVEKKRGKCGEKKKRSDCYCVCVERSRCRRLRFVLY

SEQ ID NO: 205 - protein, 57 aa, HERV-K
VYPTAPKRQRPSRTGHDDDGGFVEKKRGKCGEKKKRSDCYCVCVERSRCRRLHFVLY

SEQ ID NO: 206 - protein, 56 aa, HERV-K
LYPAAPERQRPARRGHDDGGGFFKTKRGICREKKERSDSYRLLLCLHRKGRHKRLH

SEQ ID NO: 207 - protein, 34 aa, HERV-K
VYPTAPKRKRPSRMGHDDNGGFVEKKRGNVGKRQ

SEQ ID NO: 208 - protein, 57 aa, HERV-K
VYPTAPKRQQPSRTGHDDDGGFVEKKRGKRGEKKERSDCYCVCVERSRHRRLPFVLY

SEQ ID NO: 209 - protein, 56 aa, HERV-K
VYPTAPKRQRPSRTGHDDDGGFVEKKRGKCGEKQERSNCYCVCVERSRHRRLHFVM

SEQ ID NO: 210 - protein, 46 aa, HERV-K
VYPAASETQRPARTGHDDDGGFVKKKRGICREKKVRSDCYCIYVER

SEQ ID NO: 211 - protein, 57 aa, HERV-K
VYPTAPKRQRPTRTGHDDDGGFVKKKRGKCGEKKERSGCYCACVERSRHRRLHFVLY

SEQ ID NO: 212 - protein, 55 aa, HERV-K
PTAPKRQRPSRTGHDYDGGFVEKKRGKCGEKQERSDCCCVCVERSRHRRLHFVMY

SEQ ID NO: 213 - protein, 36 aa, HERV-K
VYPTAPKRQRPSRTGHDDDGSFVIKKRGKRGEKRDQ

SEQ ID NO: 214 - protein, 57 aa, HERV-K
VYPTAPKRQRSSRMGHDDDGSFVEKKRGKCGEKKERSDCYCVYVERSRHRRLHFVLY

SEQ ID NO: 215 - protein, 57 aa, HERV-K
VYPTAPKRQRPLRMGHDDDGGFVEKKRGKCGEKKERSDCYCVCVERSRHRRLHFVLY

SEQ ID NO: 216 - protein, 57 aa, HERV-K
VYPTAPKRQRPSRMAHDDDGGFVENKSGKCGEKKERSDCYRVCVERSRHRRLHFVLY

SEQ ID NO: 217 - protein, 57 aa, HERV-K
VYPTALKRQQPSRTGHDDDGGFVEKKRGKCGEKKERSDCYCVCVERSRHRRLHFVLY

SEQ ID NO: 218 - protein, 36 aa, HERV-K
VYPTAPKRQRPSRTGHDDNGGFVEKKRGKCGEKKDQ

SEQ ID NO: 219 - protein, 57 aa, HERV-K
VYPTAPKRQRPSRTGHDDDGGFVEKKRGKCGEKKERSDCYRVCVERSRHRRLHFVLY

SEQ ID NO: 220 - protein, 51 aa, HERV-K
RRDRPWRTGHDDDGGFVEKTRGKCGEKKERSDCYCVCVERSRHRRHHFVLY

SEQ ID NO: 221 - protein, 57 aa, HERV-K
VYLAAPKRQRPSRTSHDDNGGFVKKKRGKCGEEKERSDCYCVYVERSRHKRLHFVLY

SEQ ID NO: 222 - protein, 47 aa, HERV-K
VYPAAPKRQRPSRTGHDDDGSFVKNKRENVGKRKRDQIVTVSMQKRK

SEQ ID NO: 223 - protein, 85 aa, HERV-K
EVYPTAPKRQRPSRTGHDDNGSFVKKKRGKCGEKKERSDCYCVCVERRRHRRLHFVLYQEMFFCLGMLLIYNLTPNPLLSETCAV

SEQ ID NO: 224 - protein, 41 aa, HERV-K
VYPTAPKRQRPSRTGHDERAMMTMAVLLKRKGGNAGKREIR

SEQ ID NO: 225 - protein, 57 aa, HERV-K
VYPTAPKRQRPSRMGHDDNGSFVEKKRGKCGEKKERSDCYYVCVERSRHRRLHFVLY

SEQ ID NO: 226 - protein, 43 aa, HERV-K
PGNPRRKLPQGQGHHCGEKQEGSDCYCVCVERSRHRRLHFVLH

SEQ ID NO: 227 - protein, 57 aa, HERV-K
VYATALKRQRPSRTGHDDDGGFVEKKRGKCGEKKERSDCYCVCIERSRHRRHHFVLY

SEQ ID NO: 228 - protein, 57 aa, HERV-K
VYPTSPKRQRPSRTGHDDDGGFVKKKRGKCGEKKERSDCYCVCVERSRHRRLHFVLY

SEQ ID NO: 229 - protein, 57 aa, HERV-K
VYPTAPKRQRPSRTGHDDDGGFVEKKRGKCGENKERSDCYCVCVERSRHKRLHFVLY

SEQ ID NO: 230 - protein, 57 aa, HERV-K
VYPTAPKRQQSSRTGHDDDGGFVEKKREKCGEKKERSDCYRVCVERSRHRRLHFVLY

SEQ ID NO: 231 - protein, 57 aa, HERV-K
VYPTALKRQRPWRTGHDDDGGFVEKKRGKCGEKKERSDCYCVCVERSRHRRLHFVLY

SEQ ID NO: 232 - protein, 57 aa, HERV-K
VYPTAPKRQRPSRAGHDDDRGFVEKKRGKCGEKKERSDCYCVCVERSRHRRLHFVLY

SEQ ID NO: 233 - protein, 43 aa, HERV-K
LYPTAPKRQRPLRMGHDADGGFVEKKRGKCGEKKEIRLLLCLC

SEQ ID NO: 234 - protein, 57 aa, HERV-K
VYPTAPKRQRPLRTGHDDDSGFVEKKRGKCGEKKERSDCYCVCVERSKHRRLHFVLY

SEQ ID NO: 235 - protein, 57 aa, HERV-K
VYPTAPKRQRPSRAGHDDDGGFVEKKRGKCGEKEERSDLYCVCVERSRHRRLHFVLY

SEQ ID NO: 236 - protein, 57 aa, HERV-K
VYPTAPKRQRPSRTGHDDDGSFVEKKRGKCGEKKERSDCSCVCVERSRHRRLHFVLY

SEQ ID NO: 237 - protein, 36 aa, HERV-K
VYPTAWKRQRPSRMGHDDDGGFVEKKRGKCGENRDQ

SEQ ID NO: 238 - protein, 57 aa, HERV-K
VYPTAPKRQQPSRTGHDDDGSFVKKKRGKCGEKKERSDCCCVCVERSRHRRLHFVLY

SEQ ID NO: 239 - protein, 57 aa, HERV-K
VYPTAPKRQRPWRTGHDDDGGFVKKKRGKCGEKKKRSDCYCVCVERSRHGRLRFVLY

SEQ ID NO: 240 - protein, 76 aa, HERV-K
PAWPTWRNPVSTKNTKLARHGAACLQSCREKKERSDCYCVCVEREDIRNSILTCTLNNWLAEMLLICDFAPNLSSQ

SEQ ID NO: 241 - protein, 42 aa, HERV-K
VYPTAPKRQRPSRTGHDDDGAFVEKKRGKCGEKKERSDCYCV

SEQ ID NO: 242 - protein, 57 aa, HERV-K
VYPTAPKRQQPLRTGHNDDGGFVEKKRGKYGEKKERSDCYCVCVERSRHRRLHFVLY

SEQ ID NO: 243 - protein, 57 aa, HERV-K
VYPIALKRQRPSRTGHDDDGGFVKKKRGKCGEKKERSDCYCVYVERSRHRRLHFVLY

SEQ ID NO: 244 - protein, 107 aa, HERV-K
LQVYPAAPERQRLARTDHDDDGGFVKKKRGICREKRERSDCYCVYVEREDIRDSILKKTCTLNNCFAQMLLICSFAPATLTQPGAHKNMCCMKSRFKGSRAVQDVPC

SEQ ID NO: 245 - protein, 56 aa, HERV-K
VYPTARKRQRPSRTGHDDDGGFVKKKRGKCGEKKERSDCYCVCVERSRHRRLHFVL

SEQ ID NO: 246 - protein, 46 aa, HERV-K
VYPTAPKRQRPSRTGHDDDRGFVKKKWGKMWGKKREIRLLLCLCRK

SEQ ID NO: 247 - protein, 57 aa, HERV-K
VYPTAPKRQRPLRRGHDDDGGSVKKKRGKCGEKKERSDCYCVCVERSRHKRLHFVLY

SEQ ID NO: 248 - protein, 46 aa, HERV-K
VYPAAPERQRPARRGHDDGGGFVKTKRGICREKKERSDSYCVYIER

SEQ ID NO: 249 - protein, 50 aa, HERV-K
VYPAAPERQRPARRGHDDGGGFVKMKRGICREKKERSDCYCVYIEREAIR

SEQ ID NO: 250 - protein, 95 aa, HERV-K
LQVYPAAPERQRPGRRGHDDHGGFVKKKSGKCREKRQIRLSLCLCRKGRHKRLHFEKDLYSNNCFAEMLFICSFAPATLPQSLCPNLEFTKTCVV

SEQ ID NO: 251 - protein, 82 aa, HERV-K
LQVYPAAPERQRPARRGHDDGGGFVKTKRGICREKKERSDCYCVYIEREAIRDSILKKTCTLNNCLLRCCLSVALPQPLCPN

SEQ ID NO: 252 - protein, 95 aa, HERV-K
LQVYPAAPERQRPARRDHDDHGGFVKKKSGKCREKREIRLSLCLCRKGRHKRLHFEKDLYSNNCFAEMLFICSFAPATLPQSLCPNLEFTKICVV

SEQ ID NO: 253 - protein, 95 aa, HERV-K
LQVYPAAPERQRPARRDHDDHGGFVKKKSGKCREKREIRLSLCLCRKGRHKRLHFEKDLYSNNCFAEMLFICSFAPATLPQSLCPNLEFTKTCVV

SEQ ID NO: 254 - protein, 80 aa, HERV-K
LQVYPAAPERQQPAKTGHNDYGGFVKKKRGICTAKKERSDCYCVYVEREDIRNSILTCTLNNCFAEMLLICNFAPATLPQ

SEQ ID NO: 255 - protein, 71 aa, HERV-K
LHPLSPSQLAPPQPGHPAWATPSDCHNPRAYGQDELHQVKMVECGEKQERSECHCICVERSRHGRLHFVMY

SEQ ID NO: 256 - protein, 48 aa, HERV-K
PLCPRLKQSSRLSLSSSRDCCGEKQERSDCYCVCIERSRHRRLHFVLY

SEQ ID NO: 257 - protein, 34 aa, HERV-K
LYPTAPKRQRPSRMGHDDDGGFVKKKRGKCGGKR

SEQ ID NO: 258 - protein, 57 aa, HERV-K
VYPTAPKRQRPSRRGHDDDGGFVEKKRGKCEEKKERSDCYCVCVERSRHRRLHFILY

SEQ ID NO: 259 - protein, 57 aa, HERV-K
VYPTALKRQRPSRTGHDDDGGFVEKKRGKCGEKKERSDCYCVCVERSRHRRLHFVLY

SEQ ID NO: 260 - protein, 57 aa, HERV-K
WPAAPSGRCTQQLRTGHDDNGGFVEWKGGKGGEKIEKSDGCRVCVERGRHGRFFILF

SEQ ID NO: 261 - protein, 57 aa, HERV-K
VYPTAPKRQQPSRMGHHDDGGFVEKKRGKCGEKKERSDCYCVCVERSRHRRLHFVLY

SEQ ID NO: 262 - protein, 43 aa, HERV-K
VYPTAPKRQRPSRMGHDDDGGFVEKKGGNVEKRKREQIVTVSV

SEQ ID NO: 263 - protein, 57 aa, HERV-K
VYPTAPKRQRPSRMGHDDDGSFVEKKRGKCGEKKERSDCYCVCVERSRHRRLHFVLY

SEQ ID NO: 264 - protein, 57 aa, HERV-K
VYPTAPKRQRPSRTGHDDDGSFVEKQRGKCGEKKERSDCYCVCVERSRHRRLHFVLY

SEQ ID NO: 265 - protein, 56 aa, HERV-K
VYPAAPKRQRPSRTGHDDDGGFVEKKRGKCGEKKERSDCYCVCVERSRHRRLHFVL

SEQ ID NO: 266 - protein, 57 aa, HERV-K
VYPTAPKRQRPSRRGHDDDGGFVKKKRGKCGEKKERSDCYCVCVERSRHRRLHFVLY

SEQ ID NO: 267 - protein, 57 aa, HERV-K
VYPTAPKRQRPSRMGHDDDGGFVEKKRGKCGEKKERTDCYCVYIERSRHRRLHFVLY

SEQ ID NO: 268 - protein, 57 aa, HERV-K
VYPTAPKRQRPSRTGHDDDGGFVEKKRGKCREKKERSDCYCVCVERRRHRRLHFVLY

SEQ ID NO: 269 - protein, 57 aa, HERV-K
VYPTALKRQRPLRTGHDDNGSFVEKKRGKCGEKKERSDCYCVCVERSRHRRLHFVLY

SEQ ID NO: 270 - protein, 57 aa, HERV-K
VYPTAPKRQRPSRTGHDDDGSFVEKKRGKCGEKKETSDCYCVCVERSRHRRLHFVLY

SEQ ID NO: 271 - protein, 57 aa, HERV-K
VYLTALKRQRPSRMGHDYDGSFVEKKRGKCGEKKERSDCYCVCVERSRHRRLHFVLY

SEQ ID NO: 272 - protein, 57 aa, HERV-K
VYPTVPKRQRPSRKGHEDDGCFVKKKRGKFGEKKERSDCYCVCVERSRHRRLHFVLY

SEQ ID NO: 273 - protein, 57 aa, HERV-K
MYPTPLKRQRPWRTGHDDNGGFVEKKRGKCGEKKERSDCYCVCVERSRHRRLHFVLY

SEQ ID NO: 274 - protein, 43 aa, HERV-K
VYSTAPKRQRPGRMGHDDVAVLSKRKGGNVGKRKRNQIVTVSV

SEQ ID NO: 275 - protein, 57 aa, HERV-K
VYPTAPKRQRPSRTGHDDDGGFAEKKRGKCGEKKERSDFYCVCAERSRHRRHHFVLY

SEQ ID NO: 276 - protein, 95 aa, HERV-K
LQVYPAAPERQRPARRGHDDHGGFVKKKSGKCREKREIRLSLCLCRKGRHKRLHFEKDLYSNYCFAEMLFICSFAPATLPQSLCPNLEFTKTCVV

SEQ ID NO: 277 - protein, 107 aa, HERV-K
LQVYPAAPERQQPARTGHDDYGSFVKKKRDICREKKERSDCYCVYVEKKDIRDSILKKTCTLNNCFAEMLLICSFAPATLTQPGAHKNMCCMESRLKGSRAVQDVPC

SEQ ID NO: 278 - DNA, 171 bp, HERV-K
gtgtacccaacagctccgaagagacagcaaccatcgagaacgggccatgataacgatggcagttttgtcgaaaagagaagggggaaatgtggggaaaagaaagagagatcagattgttactgtgtctgtgtagaaagaagtagacataggagactccattttgttctgtac

SEQ ID NO: 279 - DNA, 171 bp, HERV-K
gtgttcccaacagctctgaagagacagcgaccatcgagaatgggccatgatgacgatggtggttttgtcgaaaagaaaagggggaaatgtggggaaaagaaagagagatcagattgttactgtgtctgtgtagaaagaagtagacataggagactccattttgttctgtac

SEQ ID NO: 280 - DNA, 171 bp, HERV-K
gtgtacccaacagctccgaagagacagcaaccatcaagaactggccatgatgatgatggcggttttgtcaaaaagaaaagggggaaatgtggggaaaagaaagagagatcagattgttactgtgtctgtgtagaaagaagtagacataggatactccattttgttctgtac

SEQ ID NO: 281 - DNA, 195 bp, HERV-K
ctacaggtgtatccagcagctccaaagagagagcgaccagtgagaacgggccatgatgatgatggcggttttctcaaaaagaaaagggggatatgtagggaaaagaaagagagatcagacggttactgtgtctatgtagaaaaggaagacataagaaatttcattttgatctgtaccctgaacaattgctttgcc

SEQ ID NO: 282 - DNA, 126 bp, HERV-K
gtgtacccagcagctccgaagagacagcgaccatcgagaacaagccatgatgatgatggtggtttgtcgaaaaggaaatggggaaatgtggggaaaagagagatcagactgttactgtgtctgtgt

SEQ ID NO: 283 - DNA, 168 bp, HERV-K
gtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacatgggagactccattttgttatg

SEQ ID NO: 284 - DNA, 171 bp, HERV-K
gtgtacctaacagctccaaagagacagcgaccatcgagaacgggccatgatgacgatggcggttttgtcaaaaagaaaagggggaaatgtggggaaaagaaagagagatcagattgttactgtgtctgtgtagaaagaagtagacataggatactccattttgttctgtac

SEQ ID NO: 285 - DNA, 138 bp, HERV-K
gtgtatccaacagctctgaagagacagagaccaaagagaatgggccatgatgactatggcagttctgtcaaaaagaaaagggggatatgtaggggaaagaaagagagatcagactgttactgtgtctatgtagaaaag

SEQ ID NO: 286 - DNA, 171 bp, HERV-K
gtgtacccaacagctccgaagagacagcgaccattgagaacgggccatgatgacgatggcggttttgtcaaaaagaaaagggggaaatgtggggaaaagaaagagagatcagattgttactgtgtctgtgtagaaagaagtagacataggagactccattttgttctgtac

SEQ ID NO: 287 - DNA, 171 bp, HERV-K
gtgtacccaacagcaccgaagagacagcaaccatcgagaacgggccatgatgacgatggctgttttctcgaaaagaaaagggggaaatgtggggaaaagaaagagagatcagattgttactgtgtctgtgtagaaagaagtagacataggagactccattttgttctgtac

SEQ ID NO: 288 - DNA, 171 bp, HERV-K
gtgtaccgaacagctctgaagagacagcgaccatcgagaatgggccacgatgatgatggcagttttgtcgaaaagaaaagggggaaatgtggggaaaagaaagagagatcagattgttactgtgtctgtgtagaaagaagtagacataggagactccattttgttctgtac

SEQ ID NO: 289 - DNA, 171 bp, HERV-K
gtgtacccaacagctccgaagagacagcgaccatcaagaacgggccatgatgacgatggtggttttgtggaagagaaaagggggaaatgtggggcaaagaaagagagatcagattgttactgtgtctgtgtagaaagaagtagacataggagactccattttgttctgtac

SEQ ID NO: 290 - DNA, 162 bp, HERV-K
gtgtacccaacagctccaaagagacagcaaccatcgagaaacagccatgatgatgatggcggttttgtcgaaaagggggaaatgtggggaaaagaaagatcagattgttactgtgtctgtgtagaaagaagtagacataggagactccattttgttctgtac

SEQ ID NO: 291 - DNA, 135 bp, HERV-K
gtgtacccaacagctccgaaaagacagcgaccatcgagaacgggccatgatgacgatggcggttttgtcgaaaagaaaaggaggaaaagtggggaaaagagagagatcagattgttactgtgtctgtgtagaaag

SEQ ID NO: 292 - DNA, 171 bp, HERV-K
gtgtacccaacagctccaaaaagacagcgaccatcgagaacgggccatgatgacgatggcggttttgtcagaaagaaaagggggaaatgtggggaaaagaaagagagatcagattgttattgtgtctgtgtagaaagaactagacataggagattccattttgttctgtac

SEQ ID NO: 293 - DNA, 153 bp, HERV-K
gtgtacccaacagctccaaagagacagcgaccatcgagaacgggccagtatgacgatggcagttttgtcaaaaagaaaagggggagaaaagaaaagggagaaatgtggggaaaagaaagagagatcagattgttactgtgtctgtgtagaaag

SEQ ID NO: 294 - DNA, 138 bp, HERV-K
gtgtatccagcagctccgaagagacagcgaccagtgagaatgggccataatgacgatgtcagttttgtcaaaaagaaaagggggatatgtagggaaaagaaagagagatcagactgttactgtgtctatgtagaaaag

SEQ ID NO: 295 - DNA, 171 bp, HERV-K
gtgtatccaacagctccaaagagacagcgaccatcaagaatgggccatgatgactatggcggttttgtcgaaaagaaaagggggaaatgtggggaaaagaaagagagatcagattgttactgtgtctgtgtagaaagaagtagacataggagactccattttgttctgtac

SEQ ID NO: 296 - DNA, 171 bp, HERV-K
gtgtacccaacagctccaaagagacagcaaccatcgagaatgggccatgatgacgatggcggttttgtcaaaaagaaaagggggaaatgtggggaaaagaaagagagatcagattgttactttgtctgtgtagaaagaagtagacataggagactccattttgttctgtac

SEQ ID NO: 297 - DNA, 171 bp, HERV-K
gtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacataggagactccattttgttatgtac

SEQ ID NO: 298 - DNA, 171 bp, HERV-K
gtgtacccaacagctccgaagagacagcgaccatcgagaacgggccatgatgacgatggcggttttgtcgaaaagaaaagggggaaatgtggggaaaagcaagagagatcagattgtcactgtgtctgtgtagaaagaagtagacatgggagactccattttgttatgtac

SEQ ID NO: 299 - DNA, 162 bp, HERV-K
gtgtacccaacagctccgaagagacagcgaccatcgagaacgggccatgatgacagtggcggttttgtcgaaaagaaaagggggaaatgtggggaaaagaaagagagatcagattgttactgtgtctgtgtagaaagaagtagacataggagactccatttt

SEQ ID NO: 300 - DNA, 171 bp, HERV-K
gtgtacccaacagctccgaagagacagcgaccatcgagaatgggccatgatgacgatggtggttttgttgaaaagaaaagggggaaatgtggggaaaagaaagagagatcagattgttactgtgtctgtgtagaaagaagtagacataggagactccattttgttctgtac

SEQ ID NO: 301 - DNA, 171 bp, HERV-K
gtgtacccaacagctcggaagagacagcaaccatcaagaacgggccatgatgatgatggtggttttgtcgtaaagaaaagggggaaatgtggggaaaagaaagagagatcagattgttactgtgtctgtgtagaaagaagtagacataggagactccattttgttctgtac

SEQ ID NO: 302 - DNA, 171 bp, HERV-K
gtgtacccaacagctccaaagagacagcgaccatcaagaacgggccatgatgacgatggcggttttgtcgaaaagaaaagggggaaatgtggggaaaagaaagagagatcagattgttactgtgtctgtgtagaaagaagtagacataggagactccattttgttctgtac

SEQ ID NO: 303 - DNA, 135 bp, HERV-K
gtgtacccaacagctccgaagagacagcgaccatcaagaacaggccatgatgacgatggtggttttgtcgaaaaaaaagaaaagggggaaatgtggggaaaagaaagagagatcagattgttactgtgtctgtgt

SEQ ID NO: 304 - DNA, 171 bp, HERV-K
gtgtacccaacagctccgaagagacagcgaccatcgagaacaggccatgatgatgatggcggttttgtcgaaaagcaaagggggaaatgtggggaaaagaaagagagatcagattgttactgtgtctgtgtagaaagaagtagacataggagactccattttgttctgtac

SEQ ID NO: 305 - DNA, 171 bp, HERV-K
gtgtacccaacagctccgaagagacagcaaccatcgagaacgggccatgatgaggatggtggttttgttgaaaggaaaaggggaaattgtggggaaaagaaagagagatcagattgttactgtgtctgtgtagaaagaagtagacataggagactccattttgctctgtac

SEQ ID NO: 306 - DNA, 171 bp, HERV-K
gtgtacccaacagctccgaagagacagcgaccatcgagaacgggccatgatgatgatggcggttttgtcgaaaagaaaagggggaaatgtgggggaaagaatgagagatcagattgttactgtgtctgtgtagaaagaagtagacataggagactccattttgttctgtac

SEQ ID NO: 307 - DNA, 171 bp, HERV-K
gtgtacccaacagctccgaagagacagcgaccatcgagaatgggccatgatgacgatggcggttttgtcgaaaagaaaagggggaaatgtggggaaaagcaagagagatcagattgttactgtgtctgtatagaaagaagtagacataggagactccattttgttctgtac

SEQ ID NO: 308 - DNA, 195 bp, HERV-K
ctacaggtgtatccaacagctccaaagaggcagcaaccagcgagaacgggccataatgacgatggcagttttgtcaaaaagaaaagggggatatgtagggaaaagaaagagatatcagactgttactgtatctttgtagaaaaggaagacataagaaactccattttgacctgtaccgtgaacaattgttttgcc

SEQ ID NO: 309 - DNA, 165 bp, HERV-K
gtgtacccagcagctccgaagagacagcgaccgtcaagaacgagccatgatgatgatggcagttttgtcaaaaagaaaagggttatgtggggaaaagagagatcagactgttactgtgtctatgtagaaagaagtagacataagagactccattttgttctgtac

SEQ ID NO: 310 - DNA, 249 bp, HERV-K
ctacaggtgtatccagcagctccagagagacagcgaccagggagaaggggccatgatgatggtggtggttttgtcaaaacgaaaagggggatatgtaggggaaagaaagagagatcagactgttactgtgtctacatagaaagggaagacataagagactccattttgaaaaagatctgtactttaagcaattgctttgctgagatgttgttaatctgtagctttgccccagccactttgccccaacca

SEQ ID NO: 311 - DNA, 171 bp, HERV-K
ccaaagagacagcgaccatcaagaactggccatgatgacaatggtggttttgtcgaaaagaaaagggggaaatgtggggaaaagaaagagagatcagattgttactgtgtctgtgtagaaagaagtagacataggagactccactttgttctgtactaagaaaaattcttc

SEQ ID NO: 312 - DNA, 249 bp, HERV-K
ctacaggtgtatccagcagctccagagagacagcgaccagggagaaggggccatgatgacggtggtggttttgtcaaaacgaaaagggggatatgtaggggaaagaaagagagatcagactgttactgtgtctacatagaaagggaagacataagagactccattttgaaaaagaactgtactttaaacaattgctttgctgagatgtttttaatctgtagctttgccccagccacttttccccaacca

SEQ ID NO: 313 - DNA, 108 bp, HERV-K
gtgtacccaacagctccgaagagacagcgaccatcgagaacgggccatgatgacgatggcggttttgtcgaaaagaaaagggggaaatgtggggaaaagagagatcag

SEQ ID NO: 314 - DNA, 171 bp, HERV-K
gtgtacccaacagctccaaagagacagcgaccatcgagaacgggccatgatgacagtggcagttttgtcaaaaagaaaagggggaaatgtggggaaaagaaagagaggtcagattgttactgtgtctgtgtagaaagaagtagacataggagactccgttttgttctgtac

SEQ ID NO: 315 - DNA, 171 bp, HERV-K
gtgtacccaacagctccaaagagacagcgaccatcgagaatgggccatgatgacgatggcggttttgtcgaaaagcaaagggggaaatgcagggaaaagaaagagagatcagattgttactgtgtctgtgtagaaagaagttggcataggagactccattttgttctgtac

SEQ ID NO: 316 - DNA, 171 bp, HERV-K
gtgtacccaacagctctgaagagacagcaaccatcgagaacgggccatgatgacgatggcggttttgtcaaaaagaaaagggggaaatgtggggaaaagcaagagagatcagattgtcactgtgtctgtgtagaaagaagtagacataggagactccattttgttatgtac

SEQ ID NO: 317 - DNA, 171 bp, HERV-K
gtgtacccaacagctcggaagagacagagaccatcgagaacgggccatgatgacgatggcggttttgtcgaaaagaaaagggggaaatgtggggaaaagcaagagagatcagattgttactgtgtctgtgtagaaagaagtagacataggagactccattttgttctgtac

SEQ ID NO: 318 - DNA, 138 bp, HERV-K
aaaccaaggagaacaaagacacaacataccagaatctctgggacacattcaacgtgtggggaaaagcaagagagatcagattgttactgtgtctgtgtagaaagaagtagacataggagactccattttgttctgtac

SEQ ID NO: 319 - DNA, 138 bp, HERV-K
gtgtatccagcagctccagagagacagcgaccagcgagaaggggccatgatgatggtggtggttttgtcaaaacgaaaagggggatatgtagggtaaagaaagagagatcagactgttactgtgtctacatagaaagg

SEQ ID NO: 320 - DNA, 249 bp, HERV-K
ctacaggtgtatccagcagctccagagagacagcgaccagcgagaaggggccatgatgatggaggtggttttgtcaaaacgaaaatggggatatgtagggaaaagaaagagagatcagactgttactgtgtctacatagaaagggaagacataagagactccattttgaaaaagacctgtactttaaacaattgctttgctgagatgttgttaatctgtagctttgccccagccactttgccccaacca

SEQ ID NO: 321 - DNA, 171 bp, HERV-K
gtgtacccaacagctccgaagagacagcgaccatcgagaaagggccatgatgacgatggcggttttgtcgaaaagaaaagggggaaatgtggggaaaagaaagagagatcagattgttactgtgtctgtgtagaaagaagcagacataggagacaccattttgttctgtac

SEQ ID NO: 322 - DNA, 225 bp, HERV-K
tattagtctacaggtgtatacaacagctccggagagacagcgaccagcgagaacgggtcatgatgacgatggcggttttgtcaaaaagaaaagggggaaatgtagggaaaagaaagagagatcagactgtcactgtgcctatgtagaaagggaagacataagagactccattttgaaaaagacctgtactttaaacaattgctttgctgagatgttgttaatttg

SEQ ID NO: 323 - DNA, 171 bp, HERV-K
gtgtacccaacagctccaaagagacagcgaccatcgagaacgggccatgatgacgatggtggttttgtcgaaaagaaaagggagaaatgtggggaaaagaaagagagatcaaattgttactgtgtctgtgtagaaagaagcagacataggagactccattttgttctgtac

SEQ ID NO: 324 - DNA, 174 bp, HERV-K
ctgcaggtgtacccaacagctctgaagagacagcaaccatcgagaacgggccatgatgacgatggcagttttgtcgaaaagaaaagggggaaatgtggggaaaagaaagagagatcagattgttactgtgtctgtgtagaaagaagtagacataggagattccaaaaaaaaaaa

SEQ ID NO: 325 - DNA, 165 bp, HERV-K
gtgtacccaacagctctgaagagacagcgaccatcgagaacgggccatgatgacgatggcggttttgtcgaaaagaaaagggagaaatgtggggaaaagaaagatcagattgttactgtgtctgtagaaagaagtagacataggagactccattttgttctgtac

SEQ ID NO: 326 - DNA, 171 bp, HERV-K
gtgtacccaacagctccgaagagacagccaccatcaagaacgggccatgatgacgatggcggttttgtcctaaagaaaagggggaaatgtggggaaaagaaagagagatcagattgttaccatgtctgtgtagaaagaagtagacataggagacaccattttgttctgtac

SEQ ID NO: 327 - DNA, 168 bp, HERV-K
gtgtacccaacagctccgaagagacagcgaccgtcgagaacaggccatgatgacgatggcggttttgttgaaaagaaaagggggaaatgtggggaaaagaaagagagatcagattgttactgtgtctgtctagaaagaagtagacataggagactccattttgttctg

SEQ ID NO: 328 - DNA, 171 bp, HERV-K
gtgtactcaacagctccgaagagacagcgaccatcgagaacgggccatgatgacgatggcggttttgttgaaaagaaaagggggaaatgtggggaaaagaaagagagatcagattgttactgtgtctgtgtagaaagaagtagacatagcagactccattttgttctgtac

SEQ ID NO: 329 - DNA, 171 bp, HERV-K
gtgtacccaacagctccgaagagacagcgaccatcaagaacgggccatgatgacaatggcagttttgtcgaaaagaaaaaggggaaatgtggggaaaagaaagagagatcagattgttactgtgtctgtgtagaaagaagtagacataggagactccattttgttctgtac

SEQ ID NO: 330 - DNA, 171 bp, HERV-K
gtgtacccaacagctccgaagagacagcaaccatcgagaacgggccatgatgacgatggcggttttgtcaaaaagaaaaggggaaaatgtggggaaaagaaagagagatcagattgttactgtgtctgtgtagaaagaagtagacataggagactccattttgttctgtac

SEQ ID NO: 331 - DNA, 171 bp, HERV-K
gtgtacccaacagcttcgaagagacagccaccatcgggaacggaccatgatgacgatggcggttttgtcaaaaagaaaagagggaaatgtggggaaaagaaagagagatcagattgttactgtgtctgtgtagaaagaagtagacataggagactccattttcttctgtac

SEQ ID NO: 332 - DNA, 156 bp, HERV-K
ccatccgagcaaaggcccagggaaacgaatgggtgtcattctggtcctgacccgaggcacagccaggaaggtccctgtggggaaaagaaagagatatcagactgttactgtgtctatgtagaaagaagtagacataagagactccattttgttgtg

SEQ ID NO: 333 - DNA, 171 bp, HERV-K
gtgtacccaacagctccgaagagacagcgaccatcgagaacgggcagtgatgacgatggcggttttgtagaaaagaaaagggggaaatgtggggaaaagaaagagagaacagattgttactgtgtctgtgtagaaagaagtagacataggagactccattttgttctgtac

SEQ ID NO: 334 - DNA, 108 bp, HERV-K
gtgtacccaacagctccgaagaaacagcaaccatcgataatgggccatgatgacgatggcggttttgtcaaaaagaaaagggggaaatgtggggaaaagagagatcag

SEQ ID NO: 335 - DNA, 171 bp, HERV-K
gtgtacccaacagctccaaagagacagcgaccatcgagaacaggccatgatgatgatggtggttttgtcaaaaagaaaagggggaaatgtggggaaaagaaagagaaatcagattgttactgtgtctgtgtagaaagaagtagacataggagactccattttgttctgtac

SEQ ID NO: 336 - DNA, 189 bp, HERV-K
agcccctctgcccagaggccaccccgtctgggaggtgtacccaacagctcattgagaacaggccatgatgacgatggcggttttgtcgaatggagaggggggaaatgtggggaaaagatagataaatcagattgttgctgtgtctgtgtagagggaagtagacgtaggagactccattttgttctgtac

SEQ ID NO: 337 - DNA, 169 bp, HERV-K
gtgtacccaacagctccgaagagacagcgaccatcgagaacgggccatgatgacgatggcggttttgtcaaaaagaaaagggggaaatgtggggaaaagaaagagagatcagattgttactgtgtctgtgtagaaagaagtagacataggagactccattttgttctgt

SEQ ID NO: 338 - DNA, 171 bp, HERV-K
gtgtacccaacagctccgaagagacagcgaccatcgagaacgggccatgatgacgatggcggttttgtggaaaagaaaatggggaaatttggggaaaagaaagagagatcagattgttactgtgtctgtgtagaaagaagtagacataggagactccattttgttctgtac

SEQ ID NO: 339 - DNA, 171 bp, HERV-K
gtgtacccaacagctccgaagagacagcgaccattgagaatgggccatgatgacgatggcagttttgtcaaaaagaaaagggggaaatgtggggaaaagaaagagagatcagattgttactgtgtctgtgtagaaagaagtagacataggagactccattttgttctgtac

SEQ ID NO: 340 - DNA, 108 bp, HERV-K
gtgtacccaacagctccgaagagacagcgactatcgagaacgggccatgatgacgatggcggttttgtcgaaaagaaaagggggaaatgtggggaaaagaaagatcag

SEQ ID NO: 341 - DNA, 171 bp, HERV-K
gtgtacccaacagctccgaagagacagcaaccatcgagaacgggccatgatgacgatggcggttttgtcaaaaacaaaagggggaaacgtggggaaaagaaagagagatcagattgttactgtgtctgtgtagaaagaagtagacataggagacacccttttgttctgtac

SEQ ID NO: 342 - DNA, 171 bp, HERV-K
gtgtacccaacagctctgaagagacagcgaccatcgagaacgggccatgacaacgatggcggttttgtggaaaagaaaagggggaaatgtggggaaaagcaagagagatcagattgtcactgtgtctgtgtagaaagaagtagacataggagactccattttgttatgtac

SEQ ID NO: 343 - DNA, 171 bp, HERV-K
gtgcacccaacagctccgaagagacagcgaccatcaagaacgggccatgatgacaatggcagttttgtcgaaaagaaaagggggaaatgtggggaaaagaaagagagatcagattgttactgtgtctgtgtagaaagaagtagacataggagactccattttgttctgtac

SEQ ID NO: 344 - DNA, 255 bp, HERV-K
aggtgtacccaatagctccgaagagacagcgaccatcgagaacgggccatgatgacgatggcggttttgtcgaaaagaaaagggggaaatgtggggaaaagaaagagagatcagattgttactgtgtctgtgtagaaagaagtagatataggagactccattttgttctgtacttagaaaaattcttctgccttggaatgctgttaatctataaccttacccccaaccctgtgctctctgaaacatgtgctgtgt

SEQ ID NO: 345 - DNA, 171 bp, HERV-K
gtgtacccaacagctccgaagagacagcgaccattgagaatgggccatgatgacgatggcggttttgtcaaaaagaaaagggggaaatgtggggaaaagaaagagagatcagattgttactgtgtctgtgtagaaagaagtagacataggagactccattttgttctgtac

SEQ ID NO: 346 - DNA, 171 bp, HERV-K
gtgtacccaacagctccgaagagacagcgaccatcgagaacgggccatgatgacgatggtggttttgtcgaaaagaaaagggggaaatgtgggggaaagaaagagagatcagattgttcctgtgtctgtgtagaaagaagtagacataggagactccattttgttctgtac

SEQ ID NO: 347 - DNA, 171 bp, HERV-K
gtgtacccaacagctctgaagagacagcgaccatcaagaacgggccatgatgacgatggcggttttgtagaaaagaaaaggaggaaatgtggagaaaagaaagagagaacagattgttactgtgtctgtgtagaaagaagtagacataggggactccattttgttctgtac

SEQ ID NO: 348 - DNA, 169 bp, HERV-K
gtgtacccaacagctccgaagagacagcgaccatcaagaacaggccatgatgacgatggtggttttgtagaaaagaaaaggaggaaatgtggagaaaagaaagagagaacagattgttactgtgtctgtgtagaaagaagtagacataggggactccattttgttctgt

SEQ ID NO: 349 - DNA, 249 bp, HERV-K
gaggcgctccccaaatcccagacagggcggccgggcagaggcactcctcacttcctagatggggtggtggccaggcagaggcactcctcacttcccagatggggcggctggacagaggcgctccccacttcccagacggggcagccgggcagaggcactcctcacttcctcccagatgcagggcagccaggcagaggcgctcctcacctcccagatggggcggccgggcagaggcactcctcacttccc

SEQ ID NO: 350 - DNA, 171 bp, HERV-K
gtgtacccaacagcttcgaagagacagccaccatcgggaacggaccatgatgatgatggcagttttgtcaaaaagaaaagagggaaatgtggggaaaaggaagagagatcagattgttactgtgtctgtgtagaaagaagtagacataggagactccatttccttctgtac

SEQ ID NO: 351 - DNA, 171 bp, HERV-K
gtgtacccaacagctccgaagagacagcgaccatcaagaacgggccatgatgacgatggcggttttgttgaaaagaaaagggggaaatgtggggaaaagaaagagagatcagattgttactgtgtctgtgtagaaagaagtagacataggagacaccattttgttctgtac

SEQ ID NO: 352 - DNA, 171 bp, HERV-K
gtgtacccaacagctccgaagagacagcgaccattgagaatgggccatgatgacgatggcggttttgtcgaaaagaaaatggggaaatgtggggaaaagaaagagagatcagattgttactgtgtctgtgtagaaagaagtagacataggagactccattttgttctgtac

SEQ ID NO: 353 - DNA, 169 bp, HERV-K
gtgtacccaacagctccgaagagacagcgaccatggagaatgggccatgatgacgatggcggttttgttgaaaagaaaagggggaaatgtggggaaaagaaagagagatcagattgtcactgtgtctgtgtagaaagaagtagacataggagactccattttgttctgt

SEQ ID NO: 354 - DNA, 171 bp, HERV-K
acccaacagctccgaagagacagcgaccatggagaatgggccatgatgacgatggcggttttgttgaaaagaaaagggggaaatgtggggaaaagaaagagagatcagattgtcactgtgtctgtgtagaaagaagtagacataggagactccattttgttctgtactaag

SEQ ID NO: 355 - DNA, 167 bp, HERV-K
tacccaacagctctgaagagacagcgaccatcgagaacgggccatgatgactatggcagttttgtcaaaaagaaaagggggaaatgtggggaaaagaaagagagatcagattgttactgtgtctgtgtagaaagaagtagacatagcagactccattttgttctgta

SEQ ID NO: 356 - DNA, 186 bp, HERV-K
gcccccacccggcagccaccttgtctcagaggggtacccaacagctcactgagaacgggccatgatgacgatggcggttttgtcgaacagaaaagggggaaatgtcgggaaaagaaagagagatcagattgttactgtgtctgtgtagaaagaagtagacataggagactccattttgttctgtac

SEQ ID NO: 357 - DNA, 171 bp, HERV-K
gtgtacccaacagctccgaagagacagcgaccttcgagaacgggccatgatgacgatggcggttttgtcaaaaagaaaagggggaaatgtggggaaaagaaggagagatcagattgttactgtgtctgtgtagaaagaagtatacataggagactccattttgttctgtac

SEQ ID NO: 358 - DNA, 171 bp, HERV-K
gtgtacccaacagctccgaagagacagcgaccatcgagaacgggccatgatgacgatggcggttttgtcgaaaagaaaagggggaaatgtggggaaaagcaagagagatcagattgttactgtgtctgtgtagaaagaagtagacatgggagactccattttgttctgtac

SEQ ID NO: 359 - DNA, 171 bp, HERV-K
gtgtacccaacagctccgaagagacagcaaccatcgagaatgggccatgatgacaatggcggttttgttgaaaagaaaagggggaagtgtggggaaaagaaagagagatcagattattactgtgtctgtgtagaaagaagtagacataggagactccattttgttctgtac

SEQ ID NO: 360 - DNA, 171 bp, HERV-K
gtgtacccaacagctccgaagagacagcgaccatccagaacgggccatgatgacgatggtggttttgtcgaaaagaaaagggggaaatgtagggaaaagaaagagagatcagattgctactgtgtctgtgtagaaagaagtagacataggagactccattttgttctgtac

SEQ ID NO: 361 - DNA, 165 bp, HERV-K
ccatctgggaggtgtgcccaacagctcattgagaagggccatgatgacaatggcggtttggttgaatggagaagggggaagtgtggggaaaagagggagagatcggattgttgttgtgtctgtgtagagggaggcagacgtgggagactccattttgttctgtac

SEQ ID NO: 362 - DNA, 168 bp, HERV-K
tacccaacagctccgaagagacagcgaccatcgagaaagagccatgatgacgatggcggttttgttgaaaagaaaagggggaaatatggggaaaagaaagagagatcagattgttactgtgtctgtgtagaaagaagtagacacaggagactccattttgttctgtac

SEQ ID NO: 363 - DNA, 105 bp, HERV-K
tgtacccaacagctccaaagagacagcgaccatcgagaacgggccatgatgacgatggcggttttgtccaaaagaaaagggggaaatgggaaaagagagatcaga

SEQ ID NO: 364 - DNA, 204 bp, HERV-K
ctgcaggtgtacccagcagctccgaagagacagcgaccattgagaatgggtgatgacgacgatggtggttttgtcaaaaagaaaagggggaaatgtggggaaaagaaagagagatcagactgttactgtgtctatgtagaaaaggaagacataagaaactccattttgatctgtattaagaaaaattgttctgctttgcgatgc

SEQ ID NO: 365 - DNA, 111 bp, HERV-K
cgaagagagcgaccatcgagaacgagccatgatgacaacggtggttttgtcgaaaagaagggggaaatgtggggaaaagaaagagatatcagactgttactgtgtctatgt

SEQ ID NO: 366 - DNA, 171 bp, HERV-K
gtgtacccaacagctccgaagagacagcgaccatcgagaacgggccatgatgacgatggcagttttgtcaaaaacaaaagggggaaatgtggggaaaagaaagagagatcagattgttgttgtgtctgtgtagaaagaagtagacataggagactccattttgttctgtac

SEQ ID NO: 367 - DNA, 171 bp, HERV-K
gtgtacccagcagctccgaagagacagcgaccactgagaacaggccatgacgacgatggcggttttgtcgaaaagaaaagggggaaatgtggggaaaagaaacagaaatcagattgttactgtgtctgtgtagaaagagatagacataggagactccattttgttctgtac

SEQ ID NO: 368 - DNA, 171 bp, HERV-K
gtgtacccaacagctccaaagagacagcgaccatcgagaacgggccatgatgacaatggcagttttgttgaaaagaaaagggggaaatgtggggaaaagaaagagagatcagattgctactgtgtctgtgtagaaagaagtagacataggagactccattttgttctgtac

SEQ ID NO: 369 - DNA, 132 bp, HERV-K
gtgtacccaacagctccgaagagacagcaaccatcaagaacgggccatgatgacgatggcagttttgtcaaaaagaaaaggggcaaatgtggggaaaagagagatcagatgttactgtgtctgtgtagaaag

SEQ ID NO: 370 - DNA, 171 bp, HERV-K
gtgtacccaacagctccgaaaagacagcgaccattgagaacaggccatgatgacgatggcggttttgtcgaaaagaaaagggggaaatgtggggaaaagaaagagagatcagattgttactgtgtctgtgtagaaagaagtagacataagagactccattttgttctgtat

SEQ ID NO: 371 - DNA, 138 bp, HERV-K
gtgtatgcagcagctctggagagacagcgaccagcgagaaacggccatgatgatgatggcggttttgtcaaaaagaaaagggggatatatagggaaaagaaagagagatcagactgttattgtgtctatgtagaaagg

SEQ ID NO: 372 - DNA, 159 bp, HERV-K
ccgcctgagcaaaggcccagggaaatgaatgggtgtcattctggtcctgacctgaggcacagccaggaaggtccctgtggggaaaagaaagagatatcagactgttactgtgtctatgtagaaagaagtagacgtaagaggctccattttgttctgtac

SEQ ID NO: 373 - DNA, 129 bp, HERV-K
gtgtacccaatagctccgaagagacagcgaacatcaagaacgggccatgatgacaatggcggttttgtcgaaaagaaaagggaaatgtggggaaaagaaagagagatcagattgttactgtgtctgtgt

SEQ ID NO: 374 - DNA, 171 bp, HERV-K
gtgtacccaacagctccaaagagacagcaaccatcgagaacgggccatgatgacgatggcggatttgttcaaaagaaaagggggaaatgtggggaaaagaaagagcgatcagattgttactgtgtctgtgtagaaagaagtagacataggagactccattttgttctgcac

SEQ ID NO: 375 - DNA, 171 bp, HERV-K
gtgtacccaacagctccgaagagacagagaccatggagaacgggccatgatgacgatggcggttttgtcgaaaagaaaagggggaaatgtggggaaaagaaagagagatcagattgttactgtgtctgtgtagaaagaagtagacataggagactccattttgttctgtac

SEQ ID NO: 376 - DNA, 171 bp, HERV-K
cttaagagtcatcaccaggactttcttataagctaattaacaaatttgtacatggttaacaattgtttacattaaattctattggtaaagtaactgatgtgattttgttttctgaattgactctgactgatagagggaaatagacaacaaaaaagataatacttatttaca

SEQ ID NO: 377 - DNA, 171 bp, HERV-K
gtgtacccaacagctccgaagagacagcgaccatcaagaacaggccaagatgacgatggcagttttgtcgaaaagagaagggggaaatgtggggaaaagcaagagagatcagattgttactgtgtctgtgtagaaagaagtagacataggagactccattttcttatgtac

SEQ ID NO: 378 - DNA, 171 bp, HERV-K
gtgtacccaacggctccaaagagacagcgaccatcgagaacgggccatgaagacgatggcggttttgtcaaaaagaaaagggggaaatgtggggaaaagaaagagagatcagattgttactgtgtctgtgtagaaagaaatagacataggagactccattttgttctgtac

SEQ ID NO: 379 - DNA, 156 bp, HERV-K
ccgcccgagcaaaggcccagggaaatgaatgggtgtcattctggtcctgacccgaggcacagccaggaaggtccctgtggggaaaagaaagagatatcagactgttactgtgtctatgtagaaagaagtagacgtaagaggctccattttgttgtg

SEQ ID NO: 380 - DNA, 195 bp, HERV-K
gttttcaccacagccgaacagggcaggaccccagcacccgggacccagcgggactttgccaaggggatggacctggctgggccacgcggctgtttgtgtagggaaaagaaagagagatcacactgttactgtgtctatgtagaaaaggaagacataaactccattttgagctgtactaagaaaaattattttgcc

SEQ ID NO: 381 - DNA, 168 bp, HERV-K
gtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtac

SEQ ID NO: 382 - DNA, 156 bp, HERV-K
ccaccggagcaaaggcccagggaaatgaatgggtgtcattctggtcctgatccgaggcacagccaggaaggtccctgtggggaaaagaaagagatatcagactgttactgtgtctatgtagaaagaagtagacgtaagaggctccattttgttctg

SEQ ID NO: 383 - DNA, 165 bp, HERV-K
gtgtatccaacagctgtgaagagacagcgaccatcgagaacgggccatgatgacgatggcggttttgtcaaaaagaaaagggggaaatgtggggaaaagaaagagagatcagattgttactgtgtctgtgtagaaagaagtagacataggagactccattttctg

SEQ ID NO: 384 - DNA, 108 bp, HERV-K
gtgtacccaacagctctgaagagacagcgaccatcaagaacgggccatgatgatgatggcagttttgtcgaaaagaaaagggggaaatgtggggaaaagagagatcag

SEQ ID NO: 385 - DNA, 165 bp, HERV-K
ccaacagctctgaagagacagcgaccatcgagaacgggccatgatgacgatggcagttttgtagaaaagaaaagggggaaatgtggggaaaagaaagagagaacagattgttactgtgtctgtgtagaaagaagtagacataggagactccattttgttctgtac

SEQ ID NO: 386 - DNA, 138 bp, HERV-K
gtgtatccagcagctccagagagacagcgaccagcgagaaggggccataatgatggtggcggttttgtcaaaaagaaaagggggatatgcagggaaaagaaagagagatcagactgttactgtgtctacatagaaagg

SEQ ID NO: 387 - DNA, 108 bp, HERV-K
gtgtacccaacagctccgaagagacagcgaccattgagaacgggccatgatgacgatggcagttttgtcgaaaagaaaaggaggaaatgtggggaaaagagagaacag

SEQ ID NO: 388 - DNA, 171 bp, HERV-K
ctgtacccaacagctccgaagagacagcgaccatcgagaacaggccatgatgacgatagtggttttgtcgaaaagaaaagggggaaatgtggggaaaagcaagagagatcagattgttactgtgtctgtgtagaaagaagtagacataggagactccattttgttatgtac

SEQ ID NO: 389 - DNA, 222 bp, HERV-K
gacagcgaccgaccagagagaaggggccatgatgatggtggtggttttgtcaaaacgaaaagggggatatgtagggaaaagaaagagagatcagactgttactgtgtctacatagaaagggaagacataagagactccattttgaaaaagacctgtactttaaacagttgctttgacagagacagttgcttgagtgcattcatgtgtcttcttcttccacag

SEQ ID NO: 390 - DNA, 189 bp, HERV-K
agcccctctgcccagcggccaccccgtctgggaggtgtacccaatagctcattgagaacgggccatgatgccgatggcggttttgttgaatggaaaagggggaaatgtggggaaaagatagagagatcagattgttactgtgtctgtatagaaagaagtagacataggagactccattttgttctgtac

SEQ ID NO: 391 - DNA, 102 bp, HERV-K
gtgtacccaacatctccaaagagacagcgaccatcgagaacgggccatgatgacgatggcggttttgtcgaaaagaaaaggggaaatgtggggaaaagaaag

SEQ ID NO: 392 - DNA, 171 bp, HERV-K
gtgtacccaacagctccgaagagacaacgaccattgagaatgggccatggtgacgatggcggttttgtcgaaaagaaaagggggaaatgtagggaaaagaaagagagatcagattgttactgtgtctgtgtagaaagaagtagacataggagactccattttgttctgtac

SEQ ID NO: 393 - DNA, 243 bp, HERV-K
ctacaggtgtatccagcagctcaagagagacatcgaccagcgagaaggggccatgatgatggtggtggttttgtgaaaacgaaaagggggatatatagggaaaagaaagagagatcagactgttactgtgtctacacagaaagggaagacataagagactccattttgaaaaagacctgtactttaaacaattgctttgctgagatgttgttaatctgtagctttgccccggccaccttgccc

SEQ ID NO: 394 - DNA, 138 bp, HERV-K
gtgtatccagcagctacggagaaacagcgaccagcgagaacgggccatgatgacgatggcggtgttgtcaaaaagaaaagggggaaatgtagagaaaagaaagagggatcagactgtcactgtgtctatgcagaaagg

SEQ ID NO: 395 - DNA, 168 bp, HERV-K
tacccaacagctccgaagagacagcaaccttggagaacgggccttgatgaccttggcggttttttcgaaaagaaaagggggaattttggggaaaagaaagggggatcagatttttactccgtctgtgtggaaagaagtagacataggggaccccattttgttctgtac

SEQ ID NO: 396 - DNA, 171 bp, HERV-K
gtgtacccaacagctccgaagagacagcaaccatggagaacgggccatgatgaccatggcggttttgtcgaaaagaaaagggggaaatgtggggaaaagaaagagagatcagattgttactacgtctgtgtagaaagaagtagacataggagactccattttgttctgtac

SEQ ID NO: 397 - DNA, 168 bp, HERV-K
gtgtacccaacagctccgaagagacagcgaccatcgagaatgggccacgatgacgatggcggttttgttgaaaagaaaagggggaaatgtggggaaaagaaagagagatcagattgttactgtgtctgtgtagaaagtagacataggagactccattttgttctgtac

SEQ ID NO: 398 - DNA, 135 bp, HERV-K
gtgtacccaacagctccgaagagacagcgaccatcgagaacgggccatgatgatgatggcggttttgttgaaaagagaagggggaaatgtggggaaaagaaagagatcagattgttactgtgtctgtgtagaaag

SEQ ID NO: 399 - DNA, 171 bp, HERV-K
gtgtacccaactgctccaaaaagacagcaaccatcgagaacgggccatgatgacgatggcagttttgtcgaaaagaaaagggggaaatgtggggaaaagaaagaaagatcagattgttactgtgtctgtgtagaaagaagtagacataggagactccattttgttctgtac

SEQ ID NO: 400 - DNA, 138 bp, HERV-K
gtatatccagcagctccggagagacagcgaccagcgagaatgggccatgatgacgatggcggttttgtcaaaaagaaaagggggaaatgcagggaaaagaaagagagatcagactgtcacagtgtctatgtagaaaag

SEQ ID NO: 401 - DNA, 171 bp, HERV-K
gtgtacccaacagctccgaagagacagcgatcatcgagaacgggccgtgataacgatggcggttttgtcaaaaagaaaagggggaaatgtggggaaaagaaagagagatcagattgttactgtgtctgtgtaggaagaagtagacataggagactccattttgttctgtac

SEQ ID NO: 402 - DNA, 138 bp, HERV-K
gtgtatccagcagctccagagagacagcaaccagcgagaaggggtcatgatgatggtggtggttttgtcaaaaagaaaagggggatatgcagggaaaagaaagagagatcagacagttactgtgtctatatagaaagg

SEQ ID NO: 403 - DNA, 159 bp, HERV-K
gtgtatccagcagctccggagagacagcgaccagtgagaaggggccatgatgacgatggcggttttgttaaaaagaaaagggggaaatgtagggaaaagagagagatcagactgtcactgtgtctatgtagaaagggaagacataagagactccacttt

SEQ ID NO: 404 - DNA, 171 bp, HERV-K
gtgtacccaacagctccgaagagacagcgaccatcgagaacgggccatgatgacgatggcggttttgttgaaaagaaaagggggaaatgtggggaaaagaaaaagagatcagattgttattgtgtctgtgtagaaagaagtagatgtaggagactccgttttgttctgtac

SEQ ID NO: 405 - DNA, 170 bp, HERV-K
tcgagaacgggccatgatgacgatggcggttttgttgaaaagaaaagggggaaatgtggggaaaagaaaaagagatcagattgttattgtgtctgtgtagaaagaagtagatgtaggagactccgttttgttctgtactaagaaaaattcttctgccttgggatgctgtt

SEQ ID NO: 406 - DNA, 168 bp, HERV-K
ttgtatccagcagctccagagagacagcgaccagcaagaaggggccatgatgatggtggtggttttttcaaaacgaaaagggggatatgtagggaaaagaaagagagatcagactcttacagactcttactgtgtctacatagaaagggaagacataagagactccat

SEQ ID NO: 407 - DNA, 102 bp, HERV-K
gtgtacccaacagctccgaagagaaagcgaccatcgagaatgggccatgatgacaatggcggttttgtcgaaaagaaaagggggaatgtggggaaaagacag

SEQ ID NO: 408 - DNA, 171 bp, HERV-K
gtgtacccaacagctccgaagagacagcaaccatcgagaacgggccatgatgacgatggcggttttgtcgaaaagaaaagagggaagcgtggggaaaagaaagagagatcagattgttactgtgtctgtgtagaaagaagtagacataggagactcccttttgttctgtac

SEQ ID NO: 409 - DNA, 168 bp, HERV-K
gtgtacccaacagctccgaagagacagcgaccatcgagaacgggccatgatgacgatggcggttttgtcgaaaagaaaagggggaaatgtggggaaaagcaagagagatcaaattgttactgtgtctgtgtagaaagaagtagacataggagactccattttgttatg

SEQ ID NO: 410 - DNA, 138 bp, HERV-K
gtgtatccagcagcttcggagacacagcgaccggcgagaacgggacatgatgatgatggcggttttgtcaaaaagaaaagggggatatgtagggaaaagaaagtgagatcagactgttactgtatctatgtagaaagg

SEQ ID NO: 411 - DNA, 171 bp, HERV-K
gtgtacccaacagctccgaagagacagcgaccaacgagaacaggccatgatgacgatggcggttttgtcaaaaagaaaagggggaaatgtggggaaaagaaagagagatcaggttgttactgtgcctgtgtagaaagaagtagacataggagactccattttgttctgtac

SEQ ID NO: 412 - DNA, 165 bp, HERV-K
aaatgtggggaaaagcaagagcgatcagattgttgctgtgtctgtgtagaaagaagtagacataggagactccattttgttatgtactaagaaaaattcttctgccttgagattctgtgaccttacccccaaccccatgctctctgaaacatgtgctgtgtcaac

SEQ ID NO: 413 - DNA, 108 bp, HERV-K
gtgtacccaacagctccgaagagacagcgaccatcgagaacgggccatgatgacgatggcagttttgtcatcaagaaaagggggaaacgtggggaaaagagagatcag

SEQ ID NO: 414 - DNA, 171 bp, HERV-K
gtgtacccaacagctccgaagagacagcgatcatcgagaatgggccatgatgacgatggcagttttgtcgaaaagaaaagggggaaatgtggggaaaagaaagagagatcagattgttactgtgtctatgtagaaagaagtagacataggagactccattttgttctgtac

SEQ ID NO: 415 - DNA, 171 bp, HERV-K
gtgtacccaacagctccaaagagacagcgaccattgagaatgggccatgatgacgatggcggttttgtcgaaaagaaaagagggaaatgtggggaaaagaaagagagatcagattgttactgtgtctgtgtagaaagaagtagacataggagactccattttgttctgtac

SEQ ID NO: 416 - DNA, 171 bp, HERV-K
gtgtacccaacagctccgaagagacagcgaccatcgagaatggcccatgatgacgatggcggttttgtcgaaaacaaaagcgggaaatgtggggaaaagaaagagagatcagattgttaccgtgtctgtgtagaaagaagtagacataggagactccattttgttctgtac

SEQ ID NO: 417 - DNA, 171 bp, HERV-K
gtgtacccaacagctctgaagagacagcaaccatcgagaacgggccatgatgacgatggcggttttgtcgaaaagaaaagggggaaatgtggggaaaagaaagagagatcagattgttactgtgtctgtgtagaaagaagtagacacaggagactccattttgttctgtac

SEQ ID NO: 418 - DNA, 108 bp, HERV-K
gtgtaccccacagctccgaagaggcagcgaccatcgagaacgggccatgatgacaatggcggttttgtcgaaaagaaaagggggaaatgtggggaaaagaaagatcag

SEQ ID NO: 419 - DNA, 171 bp, HERV-K
gtgtacccaacagctccgaagagacagcgaccatcgagaacgggccatgatgatgatggtggttttgtcgaaaagaaaagggggaaatgtggggaaaagaaagagagatcagattgttaccgtgtctgtgtagaaagaagtagacataggagactccattttgttctgtac

SEQ ID NO: 420 - DNA, 153 bp, HERV-K
cgaagagacagaccatggagaacgggccatgatgacgatggcggttttgtcgaaaagacaagggggaaatgtggggaaaagaaagagagatcagattgttactgtgtctgtgtagaaagaagtagacataggagacaccattttgttctgtac

SEQ ID NO: 421 - DNA, 171 bp, HERV-K
gtgtacctagcagctccaaagagacagcgaccatcgaggacaagccatgatgacaatggtggttttgtcaaaaagaaaagggggaaatgtggggaagagaaagagagatcagactgttactgtgtctatgtagaaagaagtagacataagagactccattttgttctgtac

SEQ ID NO: 422 - DNA, 141 bp, HERV-K
gtgtacccagcagctccgaagagacagcgaccatcaagaacgggccatgatgacgatggcagttttgtcaaaaacaaaagggagaatgtggggaaaagaaagagagatcagattgttactgtgtctatgcagaaaaggaag

SEQ ID NO: 423 - DNA, 255 bp, HERV-K
gaggtgtacccaacagctccgaagagacagcgaccatcgagaacgggccatgatgacaatggcagttttgtcaaaaagaaaagggggaaatgtggggaaaagaaagagagatcagattgttactgtgtctgtgtagaaagaaggagacataggagactccattttgttctgtaccaagaaatgttcttctgccttgggatgctgttaatctataaccttacccctaaccccctgctctctgaaacatgtgctgtg

SEQ ID NO: 424 - DNA, 123 bp, HERV-K
gtgtacccaacagctccaaagagacagcgaccatcgagaacaggccatgatgaacgggccatgatgacgatggcggttttgttgaaaagaaaagggggaaatgcggggaaaagagagatcaga

SEQ ID NO: 425 - DNA, 171 bp, HERV-K
gtgtacccaacagctccgaagagacagcgaccatcgagaatgggccatgatgacaatggcagttttgtggaaaagaaaagggggaaatgtggggaaaagaaagagagatcagattgttactatgtctgtgtagaaagaagtagacacaggagactccattttgttctgtac

SEQ ID NO: 426 - DNA, 129 bp, HERV-K
cctgggaacccaaggagaaaactgccacaggggcagggccaccactgtggggaaaagcaagagggatcagattgttactgtgtctgtgtagaaagaagtagacataggagactccattttgttctgcac

SEQ ID NO: 427 - DNA, 171 bp, HERV-K
gtgtacgcaacagctctgaagagacagcgaccatcgagaacgggccatgatgacgatggcggttttgtcgaaaagaaaagggggaaatgtggggaaaagaaagagagatcagattgttactgtgtctgcatagaaagaagtagacataggagacaccattttgttctatac

SEQ ID NO: 428 - DNA, 171 bp, HERV-K
gtgtacccaacatctccaaagagacagagaccatcgagaacaggccatgatgacgatggtggttttgtcaaaaagaaaagggggaaatgtggggaaaagaaagagagatcagattgttactgtgtctgtgtagaaagaagtagacataggagactccattttgttctgtac

SEQ ID NO: 429 - DNA, 171 bp, HERV-K
gtgtacccaacagctccaaagagacagcgaccatcgagaacgggccatgatgatgatggcggttttgtcgaaaagaaaagggggaaatgtggggaaaacaaagagagatcagattgttactgtgtctgtgtagaaagaagtagacataagagactccattttgttctgtac

SEQ ID NO: 430 - DNA, 171 bp, HERV-K
gtgtacccaacagctccaaagagacagcaatcatccagaacgggccatgatgacgatggtggttttgtcgaaaagaaaagggagaaatgcggggaaaagaaagagagatcagattgttaccgtgtctgtgtagaaagaagtagacataggagactccattttgttctgtac

SEQ ID NO: 431 - DNA, 171 bp, HERV-K
gtgtatccaacagctctgaagagacagcgaccatggagaacgggccatgatgacgatggtggttttgtcgaaaagaaaagggggaaatgtggggaaaagaaagagagatcagattgttactgtgtctgtgtagaaagaagtagacataggagactccattttgttctgtac

SEQ ID NO: 432 - DNA, 171 bp, HERV-K
gtgtacccaacagctccgaagagacagcggccatcgagagcgggccatgatgacgatcgcggttttgttgaaaagaaaagggggaaatgtggggaaaagaaagagagatcagattgttactgtgtctgtgtagaaagaagtagacataggagactccattttgttctgtac

SEQ ID NO: 433 - DNA, 129 bp, HERV-K
ttgtacccaacagctccaaagagacagcgaccattgagaatgggccatgatgccgatggcggttttgttgaaaagaaaagggggaaatgtggggaaaagaaagagatcagattgttactgtgtctgtgc

SEQ ID NO: 434 - DNA, 171 bp, HERV-K
gtgtacccaacagctccaaagagacagcgaccattgagaacgggccatgatgacgatagtggttttgtcgaaaagaaaagggggaaatgtggggaaaagaaagagagatcagattgttactgtgtctgtgtagaaagaagtaaacataggagactccattttgttctgtac

SEQ ID NO: 435 - DNA, 171 bp, HERV-K
gtgtacccaacagctccgaagagacagcgaccatcgagagcgggccatgatgacgatggcggttttgtcgaaaagaaaagggggaaatgtggggaaaaggaagagaggtcagatttgtactgtgtctgtgtagaaagaagtagacataggagactccattttgttctgtac

SEQ ID NO: 436 - DNA, 171 bp, HERV-K
gaggtgtacccaacagctccgaagagacagcgaccatcgagaacgggccatgatgacgatggcagttttgtcgaaaagaaaagggggaaatgtggggaaaagaaagagagatcagattgttcctgtgtctgtgtagaaagaagtagacataggagactccattttgttctg

SEQ ID NO: 437 - DNA, 108 bp, HERV-K
gtgtacccaacagcttggaagagacagcgaccatcgagaatgggccatgatgacgatggcggttttgtcgaaaagaaaagggggaaatgtggggaaaacagagatcag

SEQ ID NO: 438 - DNA, 171 bp, HERV-K
gtatacccaactgctccgaagagacagcaaccatcgagaacgggccatgatgacgatggcagttttgtcaaaaagaaaagggggaaatgtggggaaaagaaagagagatcagattgttgctgtgtctgtgtagaaagaagtagacataggagactccattttgttctgtac

SEQ ID NO: 439 - DNA, 171 bp, HERV-K
gtgtacccaacagctccgaagagacagcgaccatggagaacgggccatgatgacgatggtggttttgtcaaaaagaaaagggggaaatgtggggaaaagaaaaagagatcagattgttactgtgtctgtgtagaaagaagcagacatgggagactccgttttgttctgtac

SEQ ID NO: 440 - DNA, 228 bp, HERV-K
ccagcctggccaacatggagaaatcccgtctctactaaaaatacaaaattagccaggcatggtgctgcatgcctgcaatcctgtagggaaaagaaagagagatcagactgttactgtgtctgtgtagaaagggaagacataagaaattccattttgacctgtaccttgaacaattggttggctgagatgctgttaatttgtgactttgccccaaatttgagctcacaa

SEQ ID NO: 441 - DNA, 126 bp, HERV-K
gtgtacccaacagctccgaagagacagcgaccatcgagaacgggccatgatgacgatggcgcttttgtcgaaaagaaaagggggaaatgtggggaaaagaaagagagatcagattgttactgtgtc

SEQ ID NO: 442 - DNA, 171 bp, HERV-K
gtgtacccaacagctccgaagagacagcaaccattgagaacaggccataatgacgatggcggttttgttgaaaagaaaagggggaaatatggggaaaagaaagagagatcagattgttactgtgtctgtgtagaaagaagtagacataggagactccattttgttctgtac

SEQ ID NO: 443 - DNA, 171 bp, HERV-K
gtgtacccaatagctctgaagagacagcgaccatcgagaacgggccatgacgacgatggcggttttgtcaaaaagaaaagggggaaatgtggggaaaagaaagagagatcagattgttactgtgtctatgtagaaagaagtagacataggagactccattttgttctgtac

SEQ ID NO: 444 - DNA, 247 bp, HERV-K
ctacaggtgtatccagcagctccggagagacagcggctagcgagaacggaccatgatgatgatggcggttttgtcaaaaagaaaagggggatatgtagggaaaagagagagagatcagactgttactgtgtctatgtagaaagggaagacataagagactccattttgaaaaagacctgtactttgaacaattgctttgctcagatgttgttaatttgtagttttgccccagccactttgacccaac

SEQ ID NO: 445 - DNA, 168 bp, HERV-K
gtgtacccaacagctcggaagagacagcgaccatcgagaacgggccatgatgatgatggcggttttgtcaaaaagaaaagggggaaatgtggggaaaagaaagagagatcagattgttactgtgtctgtgtagaaagaagtagacataggagactccattttgttctg

SEQ ID NO: 446 - DNA, 138 bp, HERV-K
gtgtacccaacagctccgaagagacagcgaccatcgagaacaggccatgatgacgatcgcggttttgtcaaaaagaaatgggggaaaatgtggggaaaaaaaagagagatcagattgttactgtgtctgtgtagaaag

SEQ ID NO: 447 - DNA, 171 bp, HERV-K
gtgtacccaacagctccaaagagacagcgaccattgagaaggggccatgatgacgatggtggttctgtcaaaaagaaaagggggaaatgtggggaaaagaaagagagatcagattgttactgtgtctgtgtagaaagaagtagacataagagactccattttgttctgtac

SEQ ID NO: 448 - DNA, 138 bp, HERV-K
gtgtatccagcagctccagagagacagcgaccagcgagaaggggccatgatgatggtggtggttttgtcaaaacgaaaagggggatatgtagggaaaagaaagagagatcagactcttactgtgtctacatagaaagg

SEQ ID NO: 449 - DNA, 150 bp, HERV-K
gtgtatccagcagctccagagagacagcgaccagcgagaaggggccatgatgatggtggtggttttgtcaaaatgaaaagggggatatgtagggaaaagaaagagagatcagactgttactgtgtctacatagaaagggaagccataaga

SEQ ID NO: 450 - DNA, 247 bp, HERV-K
ctacaggtgtatccagcagctccggagagacagcgaccagggagaaggggccatgatgaccatggcggttttgtcaaaaagaaaagcgggaaatgtagggaaaagagacagatcagactgtcactgtgtctatgtagaaagggaagacataagagactccattttgaaaaagacctgtactctaacaattgctttgctgagatgttgttcatttgtagctttgccccagccactttgccccagtcac

SEQ ID NO: 451 - DNA, 246 bp, HERV-K
ctacaggtgtatccagcagctccagagagacagcgaccagcgagaaggggccatgatgatggtggtggttttgtcaaaacgaaaagggggatatgtagggaaaagaaagagagatcagactgttactgtgtctacatagaaagggaagccataagagactccattttgaaaaagacctgtactttaaacaattgcttgctgagatgttgtttatctgtagctttgccccagccactttgccccaac

SEQ ID NO: 452 - DNA, 247 bp, HERV-K
ctacaggtgtatccagcagctccggagagacagcgaccagcgagaagggaccatgatgaccatggcggttttgtcaaaaagaaaagcgggaaatgtagggaaaagagagagatcagactgtcactgtgtctatgtagaaagggaagacataagagactccattttgaaaaagacctgtactctaacaattgctttgctgagatgttgttcatttgtagctttgccccagccactttgccccagtcac

SEQ ID NO: 453 - DNA, 247 bp, HERV-K; identical to SEQ ID NO: 452

SEQ ID NO: 454 - DNA, 240 bp, HERV-K
ctacaggtgtatccagcagctccagagagacagcaaccagcgaaaacaggccataatgactatggcggttttgtcaaaaagaaaagggggatatgtacggcaaagaaagagagatcagactgttactgtgtctatgtagaaagggaagacataagaaattccattttgacctgtaccttgaacaattgctttgctgagatgttgttaatttgtaactttgccccagccactttgccccaa

SEQ ID NO: 455 - DNA, 213 bp, HERV-K
ctgcacccgctgtcaccgtcacagctggccccacctcagccgggacaccctgcctgggccactccaagtgactgtcacaacccgagagcctatggccaagatgagctccaccaagtaaaaatggtggagtgtggggaaaagcaagagagatcagagtgtcactgtatctgtgtagaaagaagtagacatggaagactccattttgttatgtac

SEQ ID NO: 456 - DNA, 144 bp, HERV-K
cctctgtgtcccaggctaaagcagtcttcccgcctcagcctctcgagtagcagagactgctgcggggaaaagcaagagagatcagattgttactgtgtctgtatagaaagaagtagacataggagactccattttgttctgtac

SEQ ID NO: 457 - DNA, 102 bp, HERV-K
ctgtacccaacagctccgaagagacagcgaccatcgagaatgggccatgatgatgatggtggttttgtcaaaaagaaaagggggaaatgtgggggaaagaga

SEQ ID NO: 458 - DNA, 171 bp, HERV-K
gtgtacccaacagctccaaagagacagcgaccatcgagaaggggccatgatgacgatggcggttttgtcgaaaagaaaagggggaaatgtgaggaaaagaaagagagatcagattgttactgtgtctgtgtagaaagaagtagacataggagactccatttcattctgtac

SEQ ID NO: 459 - DNA, 171 bp, HERV-K
gtgtacccaacagctctgaagagacagcgaccatcaagaacgggccatgatgacgatggcggttttgtcgaaaagaaaagggggaaatgtggggaaaagaaagagagatcagattgttactgtgtctgtgtagaaagaagtagacataggagactccattttgttctgtac

SEQ ID NO: 460 - DNA, 171 bp, HERV-K
tggccagccgccccgtctgggaggtgtacccaacagctgagaacgggccatgatgacaatggcggttttgtggagtggaaaggggggaaaggtggggaaaagattgagaaatcggatggttgccgtgtctgtgtagaaagaggtagacatgggagatttttcattttgttc

SEQ ID NO: 461 - DNA, 171 bp, HERV-K
gtgtacccaacagctccgaagagacagcaaccatcgagaatgggccatcatgacgatggcggttttgttgaaaagaaaagggggaaatgtggggaaaagaaagagagatcagattgttactgtgtctgtgtagaaagaagtagacataggagactccattttgttctgtac

SEQ ID NO: 462 - DNA, 129 bp, HERV-K
gtgtacccaacagctccgaagagacagcgaccatcgagaatgggccatgatgacgatggcggttttgtagaaaaaaaagggggaaatgtggagaaaagaaagagagaacagattgttactgtgtctgtg

SEQ ID NO: 463 - DNA, 171 bp, HERV-K
gtgtacccaacagctccgaagagacagcgaccatcgagaatgggccatgatgacgatggcagttttgtcgaaaagaaaagggggaaatgtggggaaaagaaagagagatcagattgttactgtgtctgtgtagaaagaagtagacataggagactccattttgttctgtac

SEQ ID NO: 464 - DNA, 171 bp, HERV-K
gtgtacccaacagctccgaagagacagcgaccatcgagaacgggccatgatgacgatggcagttttgtcgaaaagcaaagggggaaatgtggggaaaagaaagagagatcagattgttactgtgtctgtgtagaaagaagtagacataggagactccattttgttctgtac

SEQ ID NO: 465 - DNA, 168 bp, HERV-K
gtgtacccagcagctccgaagagacagcgaccatcgagaacgggccatgatgacgatggcggttttgtcgaaaagaaaagggggaaatgtggggaaaagaaagagagatcagattgttactgtgtctgtgtagaaagaagtagacataggagactccattttgttctg

SEQ ID NO: 466 - DNA, 171 bp, HERV-K
gtgtacccaacagctccgaagagacagcgaccatcgagaaggggccatgatgacgatggcggttttgtcaaaaagaaaagggggaaatgtggggaaaagaaagagagatcagattgttactgtgtctgtgtagaaagaagtagacataggagactccattttgttctgtac

SEQ ID NO: 467 - DNA, 171 bp, HERV-K
gtgtacccaacagctccgaagagacagcgaccatcgagaatgggccatgatgacgatggcggttttgtcgaaaagaaaagggggaaatgtggggaaaagaaagagagaacagattgttactgtgtctatatagaaagaagtagacataggagactccattttgttctgtac

SEQ ID NO: 468 - DNA, 171 bp, HERV-K
gtgtacccaacagctccgaagagacagcgaccatccagaacgggccatgatgacgatggcggttttgttgaaaagaaaagggggaaatgtagggaaaagaaagagcgatcagattgttactgtgtctgtgtagaaagaaggagacataggagactccattttgttctgtac

SEQ ID NO: 469 - DNA, 171 bp, HERV-K
gtgtacccaacagctctgaagagacagcgaccattgagaacgggccatgatgacaatggcagttttgtcgaaaagaagagggggaaatgtggggaaaagaaagagagatcagattgttactgtgtctgtgtagaaagaagtagacataggagactccattttgttctgtac

SEQ ID NO: 470 - DNA, 171 bp, HERV-K
gtgtacccaacagctccgaagagacagcgaccatcgagaacgggccatgatgacgacggcagttttgtcgaaaagaaaaggggaaaatgtggggaaaagaaagagacatcagattgttactgtgtctgtgtagaaagaagtagacataggagactccattttgttctgtac

SEQ ID NO: 471 - DNA, 171 bp, HERV-K
gtgtacctaacagctctgaagagacagcgaccatcaagaatgggccatgattacgatggcagttttgtcgaaaagaaaaggggcaaatgtggggaaaagaaagagagatcagattgttactgtgtctgtgtagaaagaagtagacataggagactccattttgttctgtac

SEQ ID NO: 472 - DNA, 171 bp, HERV-K
gtgtacccaacagttccgaagagacagcgaccatcgagaaagggccatgaagacgatggctgttttgtcaaaaagaaaagggggaaatttggggaaaagaaagagagatcagattgttactgtgtctgtgtagaaagaagtagacataggagactccattttgttctgtac

SEQ ID NO: 473 - DNA, 171 bp, HERV-K
atgtacccaacacctctgaagagacagcgaccatggagaacgggccatgatgacaatggcggttttgtcgaaaagaaaagggggaaatgtggggaaaagaaagagagatcagattgttactgtgtctgtgtagaaagaagtagacataggagactccattttgttctgtac

SEQ ID NO: 474 - DNA, 129 bp, HERV-K
gtgtactcaacagctccgaagagacagcgaccagggagaatgggccatgatgacgtggcggttttgtcgaaaagaaaagggggaaatgtggggaaaagaaagagaaatcagattgttactgtgtctgtg

SEQ ID NO: 475 - DNA, 171 bp, HERV-K
gtgtacccaacagctccgaagagacagagaccatcaagaacgggccatgatgacgatggcggttttgccgaaaagaaaagggggaaatgtggggaaaagaaagagagatcagatttttactgtgtctgtgcagaaagaagtagacataggagacaccactttgttctgtac

SEQ ID NO: 476 - DNA, 255 bp, HERV-K
ctacaggtgtatccagcagctccggagagacagcgaccagcgagaaggggccatgatgaccatggcggttttgtcaaaaagaaaagcgggaaatgtagggaaaagagagagatcagactgtcactgtgtctatgtagaaagggaagacataagagactccattttgaaaaagacctgtactctaactattgctttgctgagatgttgttcatttgtagctttgccccagccactttgccccagtcactttgcccc

SEQ ID NO: 477 - DNA, 255 bp, HERV-K
ctacaggtgtatccagcagctccggagagacagcaaccagcgagaacgggccatgatgactatggcagttttgtcaaaaagaaaagggacatatgtagggaaaagaaagagagatcagactgttactgtgtctatgtagaaaagaaagacataagagactccattttgaaaaagacctgtactttgaacaattgctttgctgagatgttgttaatttgtagctttgccccagccactttgacccaacctggagct

SEQ ID NO: 478 - DNA, 73 bp, HERV-K
ttatgtgtatgcatatctaaaagcacagcacttaatcctttacattgtctatgatgcaaagacctttgttcac